(19) United States
(12) Patent Application Publication
(10) Pub. No.: US 2025/0243274 A1
(43) Pub. Date: Jul. 31, 2025

(54) Compositions for Redirecting Immunoglobulins to Immune Cells

(71) Applicant: Jibe Therapeutics, Inc., Somerville, MA (US)

(72) Inventor: Austin Wayne Boesch, Somerville, MA (US)

(21) Appl. No.: 18/791,355

(22) Filed: Jul. 31, 2024

Related U.S. Application Data
(60) Provisional application No. 63/516,632, filed on Jul. 31, 2023.

(51) Int. Cl. C07K 16/28; A61K 39/00; C07K 14/735

(52) U.S. Cl. CPC C07K 16/2809; C07K 14/70535; A61K 2039/505; C07K 2317/24; C07K 2317/31; C07K 2317/53; C07K 2317/565; C07K 2317/567; C07K 2317/569; C07K 2317/622; C07K 2317/94; C07K 2319/30

(57) ABSTRACT

The present invention relates to heterologous polypeptides or multimeric proteins, and nucleic acids encoding the same, comprising at least one immunoglobulin-binding domain and at least immune cell surface polypeptide or protein binding domain. Molecules of the invention can be used as a monotherapy or used to enhance standard, current or experimental antibody-based immunotherapy, such as cancer, autoimmunity or pathogenic infections.

### CROSS-REFERENCE TO RELATED APPLICATIONS

This application claims the benefit of U.S. Provisional Application No. 63/516,632, filed Jul. 31, 2023, which is hereby incorporated by reference herein in its entirety for all purposes.

### SEQUENCE LISTING

This application contains a Sequence Listing which has been submitted electronically in ST.26 formatted XML format. The Sequence Listing XML is incorporated herein by reference. Said XML files, created on Jul. 31, 2024, is named 783334_000002_SL.xml and is 157400 bytes in size.

### FIELD

The field of the invention generally relates to immunology, and more specifically relates to proteins for use in modulating disease.

### BACKGROUND

While naturally occurring antibodies, therapeutic antibodies, recombinant therapeutic antibodies and Fc receptor binding recombinant therapeutic proteins can provide great benefit in modulating disease such as cancer, autoimmunity, organ rejection, pathogenic infections such as viruses, bacteria, parasites or fungus, they are limited in the cell types they can engage based on the Fc receptors for which they have affinity and specificity.

It would be advantageous to have a mechanism for engaging antibodies, therapeutic antibodies, recombinant therapeutic antibodies and Fc receptor binding recombinant therapeutic proteins with additional cell surface proteins and cell types.

### SUMMARY OF THE INVENTION

The present invention relates to heterologous polypeptides or multimeric proteins and nucleic acids encoding the same that comprise at least one immunoglobulin-binding domain and at least one immune cell surface protein-binding domain thus creating an Immunoglobulin Redirector (IgR) molecules capable of engaging immune cells and modulating cellular activity such as effector function or suppression. IgRs of the invention can be used as a monotherapy or in combination with one or more standard, current or experimental therapeutics. such as cancer, immune disorders, or pathogenic infections.

In some embodiments, a heterologous polypeptide or multimeric protein and nucleic acids encoding the same, comprises:
(a) at least one immunoglobulin binding domain with affinity and specificity for one or more immunoglobulin isotypes, subclasses, allotypes, variants, derivatives and analogs thereof; and
(b) at least one immune cell surface protein-binding domain;
(c) optionally, at least one immunoglobulin binding domain with affinity and specificity for one or more immunoglobulin isotypes, subclasses, allotypes, variants, derivatives and analogs thereof,
(d) optionally, at least one immunoglobulin-binding domain capable of, but not limited to, binding wild-type immunoglobulin.

In some embodiments, a heterologous polypeptide or multimeric protein and nucleic acids encoding the same, comprises:
(a) at least two immunoglobulin-binding domain; and
(b) at least one immune cell surface protein-binding domain;
(c) optionally, at least two immunoglobulin binding domains with affinity and specificity for one or more immunoglobulin isotypes, subclasses, allotypes, variants, derivatives and analogs thereof,
(d) optionally, at least two immunoglobulin-binding domains capable of, but not limited to, binding wild-type immunoglobulin.

In some embodiments, a heterologous polypeptide or multimeric protein and nucleic acids encoding the same, comprises:
(a) at least one immunoglobulin-binding domain; and
(b) at least one immune cell surface protein-binding domain; and
(c) is capable of mediating immune cell activity in the presence of target specific antibodies that is substantially greater than the immune cell activity in the presence of non-specific antibodies that it is capable of binding;
(d) optionally, at least one immunoglobulin-binding domain with affinity and specificity for one or more immunoglobulin isotypes, subclasses, allotypes, variants, derivatives and analogs thereof,
(e) optionally, at least one immunoglobulin-binding domain capable of, but not limited to, binding wild-type immunoglobulin;
(f) optionally, is capable of mediating immune cell activity in the presence of target specific antibodies and non-specific antibodies, that it is capable of binding, that is substantially greater than the immune cell activity in the presence of non-specific antibodies;
(g) optionally, is capable of mediating immune cell activity in the presence of target specific antibodies and non-specific antibodies, that it is capable of binding, that is not substantially less than the immune cell activity in the presence of target specific antibodies.

In some embodiments, a heterologous polypeptide or multimeric protein and nucleic acids encoding the same, comprises:
(a) at least two immunoglobulin binding domain; and
(b) at least one immune cell surface protein-binding domain; and
(c) is capable of mediating immune cell activity in the presence of target specific antibodies that is substantially greater than the immune cell activity in the presence of non-specific antibodies that it is capable of binding;
(d) optionally, at least two immunoglobulin binding domains with affinity and specificity for one or more immunoglobulin isotypes, subclasses, allotypes, variants, derivatives and analogs thereof.
(e) optionally, at least two immunoglobulin-binding domains capable of, but not limited to, binding wild-type immunoglobulin;
(f) optionally, is capable of mediating immune cell activity in the presence of target specific antibodies and non-specific antibodies, that it is capable of binding, that is substantially greater than the immune cell activity in the presence of non-specific antibodies;
(g) optionally, is capable of mediating immune cell activity in the presence of target specific antibodies and non-specific antibodies, that it is capable of binding, that is not substantially less than the immune cell activity in the presence of target specific antibodies.

In some embodiments, or in combination of any one of the previous claims, a heterologous polypeptide and nucleic acids encoding the same, wherein:
(a) the heterologous polypeptide is a single chain with at least three domains and optional linkers between the three domains following the diagram below:
   D1-D2-D3; or
(b) the heterologous polypeptide is a single chain with at least four domains and optional linkers between the four domains following the diagram below:
   D1-D2-D3-D4

In some embodiments, or in combination of any one of the previous claims, a heterologous polypeptide or multimeric protein and nucleic acids encoding the same, wherein:
(a) at least one immunoglobulin-binding domain is derived from an antigen-binding domain, antibody or antigen-binding fragment their variants, derivatives or analogs thereof;
(b) optionally, at least one immunoglobulin-binding domain is derived from an antigen-binding domain, antibody or antigen-binding fragment their variants, derivatives or analogs thereof, including but not limited to, VH and VL pairs, ScFv, Fab, IgG, sdAb-VL, sdAb-VH, VHH or avimer, their derivatives or analogs thereof;
(c) optionally, at least one immunoglobulin-binding domain is derived from an antigen-binding domain, antibody or antigen-binding fragment their variants, derivatives or analogs thereof with affinity and specificity for one or more immunoglobulin isotypes, subclasses, allotypes, variants, derivatives or analogs thereof, including but not limited to: IgG, IgG1, IgG2, IgG3, IgG4, IgA, IgA1, IgA2, IgM, IgE, IgD.

In some embodiments, or in combination of any one of the previous claims, a heterologous polypeptide or multimeric protein and nucleic acids encoding the same, wherein:
(a) at least one immunoglobulin-binding domain is derived from an Fc receptor or Fc binder including but not limited to FcγRIII, mFcγRIV, FcγRIIa, FcγRIIb, FcγRIIc, FcγRI, mFcγRIII, mFcγRIIa, mFcγRIIb, mFcγRI, FcαRI, C1q, FcRL, FcRL5, pIgR, Fcα/μR, FcμR, FcεRI, FcεRII, FcRn, TRIM21 including allotypes, derivatives and analogs thereof;
(b) optionally, the Fc receptor is capable of binding one or more immunoglobulin subclasses, allotypes, derivatives and analogs thereof, including but not limited to: IgG, IgG1, IgG2, IgG3, IgG4, IgA, IgA1, IgA2, IgM, IgE, IgD;
(c) optionally, FcγRIIa comprises one or more mutations including but not limited R56H, K118N, T120V, L160Q and V172E, according to the residue number in SEQ ID NO: 9;
(d) optionally, FcγRIII comprises one or more mutations including but not limited S181P, K122N, T124V, Q176-E, 190R, T118K, A119L and Y134F, according to the residue number in SEQ ID NO: 1;
(e) optionally, FcγR comprises a domain from a first FcγR and a domain from an FcγR that is different from the first.

In some embodiments, or in combination of any one of the previous claims, a heterologous polypeptide or multimeric protein and nucleic acids encoding the same, comprises:
(a) at least one immune cell surface protein-binding domain derived from a natural soluble protein or ligand, including variants, derivatives and analogs thereof,
(b) optionally, at least one immune cell surface protein-binding domain derived from a natural soluble protein or ligand, including variants, derivatives and analogs thereof, including but not limited to: cytokines, chemokines, pentraxins, galectin-9, HMGB1, TGF-beta, growth factors, pattern recognition proteins, lectins, enzymes, peptides, glycans, lipids or metabolites.

In some embodiments, or in combination of any one of the previous claims, a heterologous polypeptide or multimeric protein and nucleic acids encoding the same, comprises:
(a) at least one immune cell surface protein-binding domain derived from the extracellular portion of a natural receptor or ligand, including derivatives and analogs thereof;
(b) optionally, at least one immune cell surface protein-binding domain derived from the extracellular portion of a natural receptor or ligand, including variants, derivatives and analogs thereof, including but not limited to: CD3, CD3ε, CD3δ, CD3γ, TCR, TCRα, TCRβ, TCRγ, TCRδ or combinations thereof, TCR complex, CD8, CD4, CD2, CCR8, TNFR2, CD39, CD103, Fas Ligand, MHC-I, MHC-II, MHC-G, HLA-DR, CD226, CD27, CD209, CD206, Galectin-3, LSECtin, FGL1, CD112, CD155, HVEM, CEACAM-1, CD40, CD40L, CD137, CD137L, CD28, CD56, NKG2D, NKp46, PD1, PDL1, PDL1, CTLA4, CLEC5A, CD79, BCR, OX40, OX40L, TIM3, TIM1, TIGIT, CD7, LAG3, CD11a, CD11b, CD18, CD80, CD86 or FcγRIIIa, FcγRIV, FcγRIIIb, FcγRIIa, FcγRIIc, FcγRIIb, FcγRI, C1q, FcRL5, pIgR, FcaRI, Fcα/μR, FcμR, FcεRI, FcεRII, FcRn, DC-SIGN, CD47, SIRP1α, CD96, VISTA, BTLA, B7-H3, chemokine receptors, cytokine receptors, growth factor receptors, pattern recognition receptors, enzymes, glycans, lipids, MICA, ULBP-1, ULBP-2, CD121a, CD121b, IL-18Rα, IL-18Rβ, CD25, CD122, CD132, CD124, CD213a13, CD127, CD360, CD19, CD20, CD5, IL-9R, CD213a1, CD213a2, IL-15Ra, CD123, CDw131, CDw125, CD131, CD116, CDw131, CD126, CD130, IL-11Ra, CD130, CD114, CD212, LIFR, CD130, OSMR, CDw210, IL-20Rα, IL-20Rβ, IL-14R, CDw217, CD118, CDw119, LTβR, CD120a, CD120b, BCMA, TACI, CD30, CD95 (Fas), GITR, GITRL, LTbR, TRAILR1-4, Apo3, RANK, OPG, TGF-βR1, TGF-βR2, TGF-βR3, EpoR, TpoR, Flt-3, CD117, CD115, CDw136, dectin-1, dectin-2, and dectin-3.

In some embodiments, or in combination of any one of the previous claims, a heterologous polypeptide or multimeric protein and nucleic acids encoding the same, wherein:
(a) at least one immune cell surface protein-binding domain is an antigen-binding domain, antibody or antigen-binding fragment including variants, derivatives or analogs thereof with affinity and specificity for the extracellular portion of a natural receptor or ligand;
(b) optionally, at least one immune cell surface protein-binding domain is an antigen-binding domain, antibody or antigen-binding fragment including variants, derivatives or analogs thereof with affinity and specificity for the extracellular portion of a natural receptor or ligand, including but not limited to: CD3, CD3ε, CD3δ, CD3γ, TCR, TCRα, TCRβ, TCRγ, TCRδ or combinations thereof, TCR complex, CD8, CD4, CD2, CCR8, TNFR2, CD39, CD103, Fas Ligand, MHC-I, MHC-II, MHC-G, HLA-DR, CD226, CD27, CD209, CD206, Galectin-3, LSECtin, FGL1, CD112, CD155, HVEM, CEACAM-1, CD40, CD40L, CD137, CD137L, CD28, CD56, NKG2D, NKp46, PD1, PDL1, PDL1, CTLA4, CLEC5A, CD79, BCR, OX40, OX40L, TIM3, TIM1, TIGIT, CD7, LAG3, CD11a, CD11b, CD18, CD80, CD86 or FcγRIIIa, FcγRIV, FcγRIIIb, FcγRIIa, FcγRIIc, FcγRIIb, FcγRI, C1q, FcRL5, pIgR, FcαRI, Fcα/μR, FcμR, FcεRI, FcεRII, FcRn, DC-SIGN, CD47, SIRP1α, CD96, VISTA, BTLA, B7-H3, chemokine receptors, cytokine receptors, growth factor receptors, pattern recognition receptors, enzymes, glycans, lipids, MICA, ULBP-1, ULBP-2, CD121a, CD121b, IL-18Rα, IL-18Rβ, CD25, CD122, CD132, CD124, CD213a13, CD127, CD360, CD19, CD20, CD5, IL-9R, CD213a1, CD213a2, IL-15Ra, CD123, CDw131, CDw125, CD131, CD116, CDw131, CD126, CD130, IL-11Ra, CD130, CD114, CD212, LIFR, CD130, OSMR, CDw210, IL-20Rα, IL-20Rβ, IL-14R, CDw217, CD118, CDw119, LTβR, CD120a, CD120b, BCMA, TACI, CD30, CD95 (Fas), GITR, GITRL, LTbR, TRAILR1-4, Apo3, RANK, OPG, TGF-βR1, TGF-βR2, TGF-βR3, EpoR, TpoR, Flt-3, CD117, CD115, CDw136 dectin-1, dectin-2, and dectin-3.

In some embodiments, or in combination of any one of the previous claims, a heterologous polypeptide or multimeric protein and nucleic acids encoding the same, wherein:
(a) at least one immune cell surface protein-binding domain is an antigen-binding domain, antibody or antigen-binding fragment their derivatives or analogs thereof, with affinity and specificity for the extracellular portion of a natural receptor or ligand present on, but not limited to: Lymphoid cells, Myeloid cells, T cells, B cells, NK cells, Macrophages, Monocytes, NK-T cells, Neutrophils, Dendritic cells, Basophils, Eosinophils and Mast cells;
(b) optionally, at least one immune cell surface protein-binding domain is an antigen-binding domain, antibody or antigen-binding fragment including not limited to a VH and VL pair, ScFv, Fab, IgG or sdAb-VL, sdAb-VH, VHH, their variants, derivatives or analogs thereof, with affinity and specificity for the extracellular portion of a natural receptor or ligand present on, but not limited to: Lymphoid cells, Myeloid cells, T cells, B cells, NK cells, Macrophages, Monocytes, NK-T cells, Neutrophils, Dendritic cells, Basophils, Eosinophils and Mast cells.

In some embodiments, or in combination of any one of the previous claims, a heterologous polypeptide or multimeric protein and nucleic acids encoding the same, wherein an additional region comprises:
(a) at least one half-life extension domain including but not limited to HSA, anti-HSA, their derivatives or analogs thereof; and
(b) optionally, the anti-serum albumin domain comprises, but is not limited to, one or more CDR or FR regions defined in SEQ ID NO: 558, 559, 560, 561, 562, 563, 564, 565, 566, 567, 568, 569, 570, 571, 572, 573, 574, 575, 576, 577 and 578, provided in Table 16, including variants, derivatives and analogs thereof.

In some embodiments, or in combination of any one of the previous claims, a multimeric protein and nucleic acids encoding the same, wherein additional regions of the molecule comprises:
(a) two half-life extension domains including but not limited to an Fc polypeptide derived from an immunoglobulin wherein the Fc does not substantially bind said immunoglobulin-binding region; and
(b) wherein the first and second Fc polypeptides comprise a hetero-multimerization domain wherein the hetero-multimerization domain may be a knob into hole mutation or mutations and the like.

In some embodiments, or in combination of any one of the previous claims, a multimeric protein and nucleic acids encoding the same, wherein additional regions of the molecule comprises:
(a) two half-life extension domains including but not limited to an Fc polypeptide derived from an immunoglobulin wherein the Fc does not substantially bind said immunoglobulin-binding region by selection of IgG heavy chain Fc polypeptide including, but not limited to, mutations L234A, L235A and P329A or alternatively P329G in the constant heavy chain domain 2 (EU Numbering); and
(b) wherein the first and second Fc polypeptides comprise a hetero-multimerization domain wherein the hetero-multimerization domain is selected from a knob into hole mutation or mutations and the like.

In some embodiments, or in combination of any one of the previous claims, a multimeric protein and nucleic acids encoding the same of any one of claims, wherein additional regions of the molecule comprises:
(a) two half-life extension domains including but not limited to an Fc polypeptide derived from an immunoglobulin wherein the Fc does not substantially bind said immunoglobulin-binding region by selection of IgG heavy chain Fc polypeptide including, but not limited to, the mutations L234A, L235A and P329A or alternatively P329G in the constant heavy chain domain 2 (EU Numbering);
(b) wherein the first Fc polypeptide includes, but not limited, to mutation T366W and optionally S354C; and wherein the second Fc polypeptide includes, but not limited to, T366S, L368A and Y407V and optionally Y349C in the constant heavy chain domain 3 (EU Numbering).

In some embodiments, or in combination of any one of the previous claims, a multimeric protein and nucleic acids encoding the same, wherein additional regions of the molecule comprises:
(a) one or more immunoglobulin kappa or lambda constant light chain, their variants, derivatives and analogs thereof,
(b) one or more immunoglobulin constant heavy chain domain 1 and all, none or a portion of the immunoglobulin hinge region, their variants, derivatives and analogs thereof;
(c) optionally, at least two immunoglobulin kappa constant light chains, their variants, derivatives and analogs thereof;
(d) optionally, at least two immunoglobulin lambda constant light chains, their variants, derivatives and analogs thereof;
(e) optionally, at least one immunoglobulin kappa constant light chain, and one immunoglobulin lambda constant light chain, their variants, derivatives and analogs thereof;
(f) optionally, one or more constant heavy chain domain 1 and all, none or a portion of the hinge comprises mutation C233S or the like and constant light chain C214S or the like (Kabat Numbering);
(g) optionally, one or more constant heavy chain domain 1 and all, none or a portion of the hinge comprises mutation C233S and F174C or the like and constant light chain C214S and S176C or the like (Kabat Numbering);
(h) optionally, at least a first pair of constant heavy chain and all, none or a portion of the hinge that comprises no mutation at C233 or the like and no mutation in constant light chain at C214 or the like and at least a second pair of constant heavy chain domain 1 all, none or a portion of the hinge comprises mutation C233S or the like and constant light chain C214S or the like (Kabat Numbering);
(i) optionally, at least a first pair of constant heavy chain and all, none or a portion of the hinge that contain no mutation at C233 or the like and no mutation in constant light chain at C214 or the like and at least a second pair of constant heavy chain domain 1 and all, none or a portion of the hinge comprises mutation C233S and F174C or the like and the constant light chain comprises mutation C214S and S176C or the like (Kabat Numbering);
(j) optionally, at least a first pair of constant heavy chain domain 1 and all, none or a portion of the hinge comprises mutation C233S or the like and constant light chain C214S or the like and at least a second pair of constant heavy chain domain 1 and all, none or a portion of the contains mutation C233S and F174C or the like and constant light chain C214S and S176C or the like (Kabat Numbering).

In some embodiments, or in combination of any one of the previous claims, a heterologous polypeptide or multimeric protein and nucleic acids encoding the same, wherein an additional region or regions of the molecule comprises:
(a) at least a second immunoglobulin-binding domain wherein the second immunoglobulin-binding domain is the same as the first immunoglobulin-binding domain; or
(b) at least a second immunoglobulin-binding domain wherein the second immunoglobulin-binding domain is different from the first immunoglobulin-binding domain;
(c) optionally, at least a second immunoglobulin-binding domain wherein the second immunoglobulin-binding domain is separated from the first immunoglobulin-binding domain by suitable linkers that can be of different lengths, such as from 1 amino acid (e.g., Gly) to 20 amino acids, from 2 amino acids to 15 amino acids, from 3 amino acids to 12 amino acids, including 4 amino acids to 10 amino acids, amino acids to 9 amino acids, 6 amino acids to 8 amino acids, or 7 amino acids to 8 amino acids, and may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids including but not limited to regions of the human constant heavy chain domain 1, kappa or lambda chain domains, polypeptides comprising linkers 13 amino acids or less, linkers 6 amino acids or less, the constant heavy chain domain 1 derived spacer ASTKGPSVFPLAP, ASTKGP or ASTKGPSVFPLAS, the constant kappa chain derived spacer RTVAAPSVFIFPP or RTVAAP, the constant lambda chain derived spacer SQPKAAPSVTLFP, GQPKANPTVTLFP, GQPKAAPSVTLFP, SQPKAA, GQPKAN or GQPKAA, (GGGS)₁, (GGGS)₂, (GGGS)₃, (GGGS)₄;
(d) optionally, at least a second immunoglobulin-binding domain wherein the second immunoglobulin-binding domain is the same as the first immunoglobulin-binding domain wherein the domains are not arranged head to tail if they are derived from FcγR type Fc receptors;
(e) optionally, at least a second immunoglobulin-binding domain wherein the second immunoglobulin-binding domain is the same as the first immunoglobulin-binding domain wherein the domains are arranged head to tail if they are derived from FcγR type Fc receptors.

In some embodiments, or in combination of any one of the previous claims, a heterologous polypeptide or multimeric protein and nucleic acids encoding the same, wherein an additional region of the molecule comprises:
(a) at least a second immune cell surface protein binding domain wherein the second immune cell binding domain is different from the first immune cell surface protein binding domain; or
(b) at least a second immune cell surface protein-binding domain wherein the second immune cell surface protein binding domain is the same as the first immune cell surface protein binding domain;
(c) optionally, at least a second immunoglobulin-binding domain wherein the second immunoglobulin-binding domain is separated from the first immunoglobulin-binding domain by suitable linkers that can be of different lengths, such as from 1 amino acid (e.g., Gly) to 20 amino acids, from 2 amino acids to 15 amino acids, from 3 amino acids to 12 amino acids, including 4 amino acids to 10 amino acids, amino acids to 9 amino acids, 6 amino acids to 8 amino acids, or 7 amino acids to 8 amino acids, and may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids including but not limited to regions of the human constant heavy chain domain 1, kappa or lambda chain domains, polypeptides comprising linkers 13 amino acids or less, linkers 6 amino acids or less, the constant heavy chain domain 1 derived spacer ASTKGPSVFPLAP, ASTKGP or ASTKGPSVFPLAS, the constant kappa chain derived spacer RTVAAPSVFIFPP or RTVAAP, the constant lambda chain derived spacer SQPKAAPSVTLFP, GQPKANPTVTLFP, GQPKAAPSVTLFP, SQPKAA, GQPKAN or GQPKAA, (GGGS)₁, (GGGS)₂, (GGGS)₃, (GGGS)₄;

In some embodiments, or in combination of any one of the previous claims, a heterologous polypeptide or multimeric protein and nucleic acids encoding the same, wherein a region or multiple regions of the molecule comprises:
(a) a single amino acid mutations or multiple amino acid mutations wherein the immunoglobulin binding domain does not substantially bind itself or another region or regions of the molecule;
(b) optionally the mutation of one or more amino acids in one or more constant domains of human IgG1, IgG2, IgG3 or IgG4, variants, derivatives and analogs thereof,
(c) optionally the mutation of one or more amino acids in the constant heavy chain domain 1 including, but not limited to, F122Y, P1265 and K213E (Kabat Numbering);
(d) optionally the mutation of one or more amino acids in a hinge region;
(e) optionally the mutation of one or more amino acids in a in the constant heavy chain domain 2 including, but no limited to, N276K, L309V, L234A, L235A and P329A or alternatively P329G (EU Numbering);
(f) optionally the mutation of one or more amino acids in a constant heavy chain domain 3;
(g) optionally the mutation of one or more amino acids in a human kappa or lambda domain.
(h) optionally the mutation of one or more amino acids in one or more light chain or heavy chain FR domains.

In some embodiments, or in combination of any one of the previous claims, a heterologous polypeptide or multimeric protein and nucleic acids encoding the same, wherein a region or multiple regions of the molecule comprises:
(a) at least one immunoglobulin binding domain comprising, but not limited to, one or more CDR or FR regions defined in SEQ ID NO: 579 through SEQ ID NO: 718, provided in Table 17, including variants, derivatives and analogs thereof, or
(b) at least one immunoglobulin binding domain consisting of anti-Fc AVIG, SEQ ID NO: 132, variants, derivatives and analogs thereof.

In some embodiments, or in combination of any one of the previous claims, a heterologous polypeptide or multimeric protein and nucleic acids encoding the same, wherein a region or multiple regions of the molecule comprises:
(a) at least one immune cell surface protein binding domain comprising, but not limited to, one or more CDR or FR regions defined in SEQ ID NO: 719 through SEQ ID NO: 1558, provided in Tables 18, 19, 20, 21, 22, 23, 24, 25, 26, 27 and 28, including variants, derivatives and analogs thereof.

In some embodiments, or in combination of any one of the previous claims, a heterologous polypeptide or multimeric protein and nucleic acids encoding the same, wherein an additional region of the molecule comprises a free cysteine at or near the C-terminus.

In some embodiments, or in combination of any one of the previous claims, a heterologous polypeptide or multimeric protein and nucleic acids encoding the same, wherein an additional region of the molecule comprises a covalently linked PEG-lipid.

In some embodiments, or in combination of any one of the previous claims, a heterologous polypeptide or multimeric protein and nucleic acids encoding the same, comprising:
(a) at least one immunoglobulin binding domain that blocks immunoglobulins from binding one or more of their cognate receptors; or
(b) at least one immunoglobulin binding domain that does not block immunoglobulins from binding one or more of their cognate receptors; or
(c) optionally, at least two immunoglobulin binding domains that block immunoglobulins from binding one or more of their cognate receptors; or
(d) optionally, at least two immunoglobulin binding domains that do not block immunoglobulins from binding one or more of their cognate receptors; or
(e) optionally, at least two immunoglobulin binding domains wherein a first immunoglobulin binding domain blocks immunoglobulins from binding one or more of their cognate receptors and a second immunoglobulin binding domain that does not block immunoglobulins from binding one or more of their cognate receptors.

In some embodiments, a multimeric protein and nucleic acids encoding the same, wherein regions of the molecule comprises:
(a) one or more immunoglobulin kappa or lambda constant light chain, their variants, derivatives and analogs thereof,
(b) one or more immunoglobulin constant heavy chain domain 1 and all, none or a portion of the immunoglobulin hinge region, their variants, derivatives and analogs thereof;
(c) optionally, at least two immunoglobulin kappa constant light chains, their variants, derivatives and analogs thereof;
(d) optionally, at least two immunoglobulin lambda constant light chains, their variants, derivatives and analogs thereof;
(e) optionally, at least one immunoglobulin kappa constant light chain, and one immunoglobulin lambda constant light chain, their variants, derivatives and analogs thereof;
(f) optionally, one or more constant heavy chain domain 1 and all, none or a portion of the hinge comprises mutation C233S or the like and constant light chain C214S or the like (Kabat Numbering);
(g) optionally, one or more constant heavy chain domain 1 and all, none or a portion of the hinge comprises mutation C233S and F174C or the like and constant light chain C214S and S176C or the like (Kabat Numbering);
(h) optionally, at least a first pair of constant heavy chain and all, none or a portion of the hinge that comprises no mutation at C233 or the like and no mutation in constant light chain at C214 or the like and at least a second pair of constant heavy chain domain 1 all, none or a portion of the hinge comprises mutation C233S or the like and constant light chain C214S or the like (Kabat Numbering);
(i) optionally, at least a first pair of constant heavy chain and all, none or a portion of the hinge that contain no mutation at C233 or the like and no mutation in constant light chain at C214 or the like and at least a second pair of constant heavy chain domain 1 and all, none or a portion of the hinge comprises mutation C233S and F174C or the like and the constant light chain comprises mutation C214S and S176C or the like (Kabat Numbering);
(j) optionally, at least a first pair of constant heavy chain domain 1 and all, none or a portion of the hinge comprises mutation C233S or the like and constant light chain C214S or the like and at least a second pair of constant heavy chain domain 1 and all, none or a portion of the contains mutation C233S and F174C or the like and constant light chain C214S and S176C or the like (Kabat Numbering).

In some embodiments, or in combination of any one of the previous claims, a multimeric protein and nucleic acids encoding the same, wherein additional regions of the molecule comprise:
(a) two half-life extension domains including but not limited to an Fc polypeptide derived from an immunoglobulin wherein the first and second Fc polypeptides comprise a hetero-multimerization domain wherein the hetero-multimerization domain is selected from a knob into hole mutation or mutations and the like; and
(b) optionally, wherein the first Fc polypeptide includes, but is not limited to, mutation T366W and optionally S354C; and wherein the second Fc polypeptide includes, but is not limited to, T366S, L368A and Y407V and optionally Y349C in the constant heavy chain domain 3 (EU Numbering);
(c) optionally, wherein the Fc does not substantially bind one or more of its cognate Fc receptors by selection of IgG heavy chain Fc polypeptide;
(d) optionally, wherein the Fc does not substantially bind one or more of its cognate Fc receptors by selection of IgG heavy chain Fc polypeptide including, but no limited to, mutation L234A, L235A and P329A or alternatively P329G in the constant heavy chain domain 2 (EU Numbering).

In some embodiments, or in combination of any one of the previous claims, a method using a heterologous polypeptide or multimeric protein and nucleic acids encoding the same of any one of claims 1-26 to treat disease as:
(a) a monotherapy or;
(b) in combination with one or more standard, current or experimental therapeutics to treat cancer, immune disorders and pathogenic infections.

In some embodiments, or in combination of any one of the previous claims, a method using a heterologous polypeptide or multimeric protein and nucleic acids encoding the same of any one of claims 1-27 to treat disease as:
(a) a monotherapy capable of binding endogenous immunoglobulins; or
(b) in combination with one or more standard, current or experimental therapeutics to treat cancer, immune disorders and pathogenic infections.

In some embodiments, or in combination of any one of the previous claims, a kit comprising heterologous polypeptide or multimeric protein and nucleic acids encoding the same.

### BRIEF DESCRIPTION OF THE DRAWINGS

FIG. 1. Depiction of Immunoglobulins (Ig) mediating effector function(s) by bridging diseased cells (ex cancer, pathogen infection, self-binding autoimmune driving Ig) to immune cells via engagement with cell surface antigens (Ag) on the diseased cell and Fc receptors (FcR) expressed on the immune cells.

FIG. 2A. Depiction of an IgR fusion protein comprising an Immunoglobulin-binding domain (IgBD) and immune cell surface protein-binding domain (ICBD) comprising an immunoglobulin redirector molecule with an optional linker. FIG. 2B. Depiction of an IgR engaging an immunoglobulin binding a diseased cell and redirecting it to an immune cell surface protein. FIG. 2C. Depiction of an IgR comprising two immunoglobulin-binding domains and one immune cell binding domain with optional linkers. FIG. 2D. Depiction of an IgR comprising one immunoglobulin-binding domain and two immune cell binding domains with optional linkers.

FIG. 3A. Depiction of an IgR comprising a half-life extension domain fused to an immunoglobulin binding domain and one immune cell binding domain with optional linkers. FIG. 3B. Depiction of an IgR comprising a half-life extension domain fused to two immunoglobulin binding domains and one immune cell binding domain with optional linkers. FIG. 3C. Depiction of an IgR comprising a half-life extension domain fused to one immunoglobulin binding domain and two immune cell binding domains with optional linkers.

FIG. 4A. Depiction of an IgR comprising an immunoglobulin-binding domain fused to an Fc attached to another Fc with a heavy-chain/light chain comprising immune cell binding domain (VH/VL pair attached to their corresponding constant heavy/light chains). FIG. 4B. Depiction of an IgR comprising an immunoglobulin-binding domain fused to an Fc attached to another Fc with a heavy-chain/light chain comprising two immune cell binding domains (one VH/VL pair attached to their corresponding constant heavy and light chains and one VH/VL pair as an ScFv fused to the C-terminus of the constant light chain). FIG. 4C. Depiction of an IgR comprising an immunoglobulin-binding domain fused to an Fc attached to another Fc with a heavy-chain comprising immune cell binding domain (VH/VL pair as an ScFv attached to the N-terminus of a constant heavy chain). FIG. 4D. Depiction of an IgR comprising an immunoglobulin-binding domain fused to an Fc attached to another Fc with a heavy-chain comprising immune cell binding domain and a 2nd immunoglobulin-binding domain (an immunoglobulin-binding domain fused to an VH/VL pair as an ScFv attached to the N-terminus of a constant heavy chain and). FIG. 4E. Depiction of an IgR comprising one immunoglobulin-binding domain fused to the N-terminus of a constant heavy chain, a second immunoglobulin-binding domain fused to the N-terminus of a constant light chain on the same Fc attached to another Fc with a heavy-chain/light chain comprising immune cell binding domain (VH/VL pair attached to their corresponding constant heavy/light chains). FIG. 4F. Depiction of an IgR comprising one immunoglobulin-binding domain fused to the N-terminus of a constant heavy chain via an optional linker, a second immunoglobulin-binding domain fused to the N-terminus of a constant light chain via an optional linker on the same Fc attached to another Fc with a heavy-chain/light chain comprising one immune cell binding domain (VH/VL pair attached to their corresponding constant heavy/light chains) and a second immune cell binding domain fused to the N-terminus of the constant light chain via an optional linker. FIG. 4G. Depiction of an IgR comprising an immunoglobulin-binding domain fused to an Fc via an optional linker and attached to another Fc with a heavy-chain/light chain comprising of immune cell binding domain (VH/VL pair attached to their corresponding constant heavy/light chains) and 2nd immunoglobulin binding domain fused to the C-terminus of the constant light chain via an optional linker. FIG. 4H. Depiction of an IgR comprising an immunoglobulin-binding domain (VH/VL pair attached to their corresponding full length constant heavy/light chains) attached to another Fc with a heavy-chain/light chain comprising of immune cell binding domain (VH/VL pair attached to their corresponding full length constant heavy/light chains). FIG. 4I. Depiction of an IgR comprising two immunoglobulin-binding domains with a first VH/VL pair attached to their corresponding full length constant heavy/light chains and a second VH/VL attached to the N-terminus of the first via an optional linker with it's Fc attached to another Fc with a heavy-chain/light chain comprising of immune cell binding domain (VH/VL pair attached to their corresponding full length constant heavy/light chains). FIG. 4J. Depiction of an IgR comprising two immunoglobulin-binding domains with a first VH/VL pair attached to their corresponding full length constant heavy/light chains) attached to another Fc with a heavy-chain/light chain comprising a second immunoglobulin-binding domains (VH/VL pair attached to their corresponding full length constant heavy/light chains) with a immune cell binding domain attached to the C-terminus of one of the light chains of the immunoglobulin binding domains by an optional linker.

FIG. 5A Depiction of an IgR comprising a Fab fusion with an immune cell binding domain VH/VL pair attached to their corresponding constant heavy chain 1 and constant light chains and one immunoglobulin binding domain fused to the C-terminus of the constant light chain via an optional linker. FIG. 5B. Depiction of an IgR comprising a Fab fusion with an immune cell binding domain VH/VL pair attached to their corresponding constant heavy chain 1 and constant light chains and one immunoglobulin binding domain fused to the C-terminus of the constant heavy chain and one immunoglobulin domain fused to the C-terminus of the constant light chain via optional linkers. FIG. 5C. Depiction of an IgR comprising a Fab fusion with an immunoglobulin binding domain VH/VL pair attached to their corresponding constant heavy chain 1 and constant light chains and one immune cell binding domain fused to the C-terminus of the constant light chain via an optional linker. FIG. 5D. Depiction of an IgR comprising a Fab fusion with an immunoglobulin binding domain attached to the N-terminus of the constant heavy chain 1, one immune cell binding domain fused to the C-terminus of the constant light chain via an optional linker. FIG. 5E. Depiction of an IgR comprising a Fab fusion with one immunoglobulin binding domain attached to the N-terminus of the constant heavy chain 1, a 2nd immunoglobulin binding domain attached to the N-terminus of the constant light chain and one immune cell binding domain fused to the C-terminus of the constant light chain via an optional linker. FIG. 5F. Depiction of an IgR comprising a Fab fusion with an immunoglobulin binding domain attached to the N-terminus of the constant heavy chain 1, one immune cell binding domain fused to the C-terminus of the constant heavy chain via an optional linker and a 2nd immune cell binding domain fused to the C-terminus of the constant light chain via an optional linker. FIG. 5G. Depiction of an IgR comprising a Fab fusion with one immunoglobulin binding domain attached to the N-terminus of the constant heavy chain 1, a 2nd immunoglobulin binding domain attached to the N-terminus of the constant light chain, one immune cell binding domain fused to the C-terminus of the constant heavy chain via an optional linker and a 2nd immune cell binding domain fused to the C-terminus of the constant light chain via an optional linker. FIG. 5H. Depiction of an IgR comprising a Fab fusion with an immunoglobulin binding domain VH/VL pair attached to their corresponding constant heavy chain 1 and constant light chains and one immune cell binding domain fused to the C-terminus of the constant light chain via an optional linker and a 2nd immune cell binding domain fused to the C-terminus of the constant heavy chain via an optional linker. FIG. 5I. Depiction of an IgR comprising a Fab fusion with two immunoglobulin binding domains with a first VH/VL pair attached to their corresponding constant heavy chain 1 and constant light chains and second VH/VL pair attached to the N-terminus of the first VH/VL pair via an optional linker; and one immune cell binding domain fused to the C-terminus of the constant light chain via an optional linker; and a half-life extension domain fused to the C-terminus of the constant heavy chain via an optional linker. FIG. 5J. Depiction of an IgR comprising a Fab fusion with an immune cell binding domain VH/VL pair attached to their corresponding constant heavy chain 1 and constant light chains and one immunoglobulin binding domain fused to the C-terminus of the constant heavy chain and one immunoglobulin domain fused to the C-terminus of the constant light chain via optional linkers; and with a half-life extension domain attached to the C-terminus of one of the immunoglobulin domains via an optional linker. FIG. 5K. Depiction of an IgR comprising a Fab fusion with an immune cell binding domain VH/VL pair attached to their corresponding constant heavy chain 1 and constant light chains and one immunoglobulin binding domain fused to the N-terminus of the variable heavy chain and one immunoglobulin domain fused to the N-terminus of the variable light chain via optional linkers. FIG. 5L. Depiction of an IgR comprising a Fab fusion with an immune cell binding domain VH/VL pair attached to their corresponding constant heavy chain 1 and constant light chains and one immunoglobulin binding domain fused to the N-terminus of the variable heavy chain via an optional linker; one immunoglobulin domain fused to the N-terminus of the variable light chain via an optional linker; and one immunoglobulin domain fused to the C-terminus of one of the constant chains via an optional linker.

FIG. 6A. Depiction of an IgR comprising a Fab fusion with an immune cell binding domain VH/VL pair attached to their corresponding constant heavy chain 1 and constant light chains and one immunoglobulin binding domain fused to the C-terminus of the constant light chain via an optional linker and a PEG-lipid covalently attached to the C-terminus of the constant heavy chain 1. FIG. 6B. Depiction of an IgR comprising a Fab fusion with an immunoglobulin binding domain VH/VL pair attached to their corresponding constant heavy chain 1 and constant light chains and one immune cell binding domain fused to the C-terminus of the constant light chain via an optional linker and a PEG-lipid covalently attached to the C-terminus of the constant heavy chain 1. FIG. 6C. Depiction of an IgR comprising a Fab fusion with an immunoglobulin binding domain attached to the N-terminus of the constant heavy chain 1 via an optional linker, one immune cell binding domain VH/VL pair ScFv fused to the C-terminus of the constant light chain via an optional linker and a PEG-lipid covalently attached to the C-terminus of the constant heavy chain 1. FIG. 6D. Depiction of an IgR comprising a Fab fusion with one immunoglobulin binding domain attached to the N-terminus of the constant heavy chain 1 via an optional linker, a 2nd immunoglobulin binding domain attached to the N-terminus of the constant light chain via an optional linker, one immune cell binding domain fused to the C-terminus of the constant light chain via an optional linker and a PEG-lipid covalently attached to the C-terminus of the constant heavy chain 1. FIG. 6E. Depiction of an IgR comprising an immunoglobulin-binding domain fused to the N-terminus of an immune cell binding domain VH/VL pair ScFv and a PEG-lipid covalently attached to the C-terminus of the ScFv. FIG. 6F. Depiction of an IgR comprising a Fab fusion with one immune cell binding domain VH/VL ScFv attached to the N-terminus of the constant heavy chain 1 via an optional linker, a 2nd immune cell binding domain VH/VL ScFv attached to the N-terminus of the constant light chain, one immunoglobulin binding domain fused to the C-terminus of the constant light chain via an optional linker and a PEG-lipid covalently attached to the C-terminus of the constant heavy chain 1. FIG. 6G. Depiction of an IgR comprising a Fab fusion with one immunoglobulin binding domain attached to the N-terminus of the constant heavy chain 1, an immune cell binding domain VH/VL pair ScFv attached to the N-terminus of the constant light chain, a 2nd immune cell binding domain VH/VL pair ScFv fused to the C-terminus of the constant light chain via an optional linker and a PEG-lipid covalently attached to the C-terminus of the constant heavy chain 1.

FIG. 7A. Depiction of an IgR comprising an Fc-binding domain and T cell receptor complex-binding domain capable of redirecting immunoglobulins to T cells. FIG. 7B. Depiction of FcB/αTCR IgR molecule augmenting Ig mediated effector function against cancer cells by recruiting and driving T cell mediated effector function.

FIG. 8A, FIG. 8C, FIG. 8E, FIG. 8G, FIG. 8I, FIG. 8K display the SDS-PAGE gels for reducing (R) and non-reducing IgRs. FIG. 8B, FIG. 8D, FIG. 8F, FIG. 8H, FIG. 8J, FIG. 8L display the size exclusion chromatography by high pressure liquid chromatography (HPLC-SEC) chromatograms for IgRs.

FIG. 9A, FIG. 9C, FIG. 9E, FIG. 9G, FIG. 9I displays the SDS-PAGE gels for reducing (R) and non-reducing IgRs. FIG. 9B, FIG. 9D, FIG. 9F, FIG. 9H, FIG. 9J displays the size exclusion chromatography by high pressure liquid chromatography (HPLC-SEC) chromatograms for IgRs.

FIG. 10. Depiction of a T cell activation assay using a co-culture of a target cancer cell line and an engineered T cell line, Jurkat NFAT-luc, that expresses luciferase when it's t cell receptors are crosslinked by combining target cell binding antibodies with Fc-binding/T cell receptor binding fusion proteins.

FIG. 11. Verification of T cell activation assay using co-culture of CD20+ Raji cells and Jurkat NFAT-Luc cells in all wells where cells alone and anti-CD20 Rituximab are negative controls and with anti-CD20/anti-CD3 Mosunetuzumab is a positive control.

FIG. 12. Verification of T cell activation assay using co-culture of CD20+ Raji cells and Jurkat NFAT-Luc cells in all wells with anti-CD20/anti-CD3 Mosunetuzumab is a positive control.

FIG. 13. Evaluation of T cell activation from FcγRIIIA/anti-CD3 or FcγRIIIA/anti-TCR fusion proteins combined with and without Rituximab (Rtx) when co-cultured with CD20+ Raji B cells and Jurkat-NFAT-Luc T cells.

FIG. 14. Evaluation of T cell activation from FcγRIIIA/anti-CD3 fusion with or without half-life extension strategies combined with and without Rituximab (Rtx) when co-cultured with CD20+ Raji B cells and Jurkat-NFAT-Luc T cells.

FIG. 15. Evaluation of T cell activation from FcγRIIIA/anti-CD3 or anti-Fc/anti-CD3 fusions combined with and without Rituximab when co-cultured with CD20+ Raji B cells and Jurkat-NFAT-Luc T cells.

FIG. 16. Evaluation of T cell activation from FcγRIIIA/anti-CD3 or FcγRIIIA/anti-TCR fusion proteins combined with Trastuzumab (Tras) or Rituximab (Rtx) when co-cultured with HER2+ SKBR3 breast cancer cells and Jurkat-NFAT-Luc T cells.

FIG. 17. Evaluation of T cell activation from FcγRIIIA/anti-CD3 fusion with or without half-life extension strategies combined with Trastuzumab (Tras) or Rituximab (Rtx) when co-cultured with HER2+ SKBR3 breast cancer cells and Jurkat-NFAT-Luc T cells.

FIG. 18. Evaluation of T cell activation from FcγRIIIA/anti-CD3 or anti-Fc/anti-CD3 fusions combined with Trastuzumab (Tras) or Rituximab (Rtx) when co-cultured with HER2+ SKBR3 breast cancer cells and Jurkat-NFAT-Luc T cells.

FIG. 19A and FIG. 19B. Display of SDS-PAGE gels images for Reduced (R) and Non-reduced (NR) IgRs from Example 18 and Marker (M) in kDa.

FIG. 20. Display of chromatograms from size exclusion chromatography using a high pressure liquid chromatography system (HPLC-SEC) for IgRs from Example 18.

FIG. 21. Evaluation of T cell activation for anti-CD3 IgRs combined with Rituximab (Rtx) or Trastuzumab (Trz) when co-cultured with CD20+ Raji B cells and Jurkat-NFAT-Luc T cells in Example 18.

FIG. 22A and FIG. 22B. Display of SDS-PAGE gels images for Reduced (R) and Non-reduced (NR) IgRs from Example 19 and Marker (M) in kDa.

FIG. 23. Display of chromatograms from size exclusion chromatography using a high pressure liquid chromatography system (HPLC-SEC) for IgRs from Example 19.

FIG. 24. Evaluation of T cell activation for anti-CD3 IgRs combined with Rituximab (Rtx) or Trastuzumab (Trz) when co-cultured with CD20+ Raji B cells and Jurkat-NFAT-Luc T cells in Example 19.

FIG. 25. Evaluation of T cell activation for anti-CD3 IgRs combined with Rituximab (Rtx) or Trastuzumab (Trz) or Rituximab and Trastuzumab when co-cultured with CD20+ Raji B cells and Jurkat-NFAT-Luc T cells in Example 20.

FIG. 26A and FIG. 26B. Display of SDS-PAGE gels images for Reduced (R) and Non-reduced (NR) IgRs from Example 21 and Marker (M) in kDa.

FIG. 27. Display of chromatograms from size exclusion chromatography using a high pressure liquid chromatography system (HPLC-SEC) for IgRs from Example 21.

FIG. 28. Evaluation of T cell activation assay for anti-CD3 IgRs combined with Rituximab (Rtx) or Trastuzumab (Trz) or Rituximab and Trastuzumab when co-cultured with CD20+ Raji B cells and Jurkat-NFAT-Luc T cells in Example 21

FIG. 29A and FIG. 29B display of SDS-PAGE gels images for Reduced (R) and Non-reduced (NR) IgRs from Example 22 and Marker (M) in kDa.

FIG. 30. Display of chromatograms from size exclusion chromatography using a high pressure liquid chromatography system (HPLC-SEC) for IgRs from Example 22.

FIG. 31. Evaluation of T cell activation for anti-CD3 IgRs combined with Rituximab (Rtx) or Trastuzumab (Trz) when co-cultured with CD20+ Raji B cells and Jurkat-NFAT-Luc T cells in Example 22.

FIG. 32. Evaluation of T cell activation for anti-CD3 IgRs combined with Rituximab (Rtx) or Trastuzumab (Trz) when co-cultured with CD20+ Raji B cells and Jurkat-NFAT-Luc T cells in Example 22.

FIG. 33. Evaluation of T cell activation for anti-CD3 IgRs combined with Rituximab (Rtx) or Trastuzumab (Trz) or Rituximab and Trastuzumab when co-cultured with CD20+ Raji B cells and Jurkat-NFAT-Luc T cells in Example 22.

FIG. 34. Evaluation of T cell activation for anti-CD3 IgRs alone or combined anti-CD3 OKT3 mIgG2a; or Rituximab (Rtx) alone; or anti-CD3 OKT3 mIgG2a alone when co-cultured with CD20+ Raji B cells and Jurkat-NFAT-Luc T cells in Example 22.

FIG. 35. Evaluation of T cell co-stimulation for anti-CD28 IgRs combined with Rituximab (Rtx) or Trastuzumab (Trz) when co-cultured with CD20+ Raji B cells and Jurkat-NFAT-Luc T cells in wells pre-coated with 1 μg/mL OKT3 in Example 22.

FIG. 36A, FIG. 36B, and FIG. 36C display of SDS-PAGE gels images for Reduced (R) and Non-reduced (NR) IgRs from Example 23 and Marker (M) in kDa.

FIG. 37A and FIG. 37B display of chromatograms from size exclusion chromatography using a high pressure liquid chromatography system (HPLC-SEC) for IgRs from Example 23.

FIG. 38. Evaluation of T cell activation for anti-CD3 IgRs combined with Rituximab (Rtx) or Trastuzumab (Trz) or Rituximab and Trastuzumab when co-cultured with CD20+ Raji B cells and Jurkat-NFAT-Luc T cells in Example 23.

FIG. 39. Evaluation of T cell activation for anti-CD3 IgRs combined with Rituximab (Rtx) or Trastuzumab (Trz) or Rituximab and Trastuzumab when co-cultured with CD20+ Raji B cells and Jurkat-NFAT-Luc T cells in Example 23.

FIG. 40. Evaluation of T cell activation for anti-CD3 IgRs combined with Rituximab (Rtx) or Trastuzumab (Trz) or Rituximab and Trastuzumab when co-cultured with CD20+ Raji B cells and Jurkat-NFAT-Luc T cells in Example 23.

FIG. 41A, FIG. 41B, and FIG. 41C display of SDS-PAGE gels images for Reduced (R) and Non-reduced (NR) IgRs from Example 24 and Marker (M) in kDa.

FIG. 42A and FIG. 42B display of chromatograms from size exclusion chromatography using a high pressure liquid chromatography system (HPLC-SEC) for IgRs from Example 24.

FIG. 43. Evaluation of T cell activation for anti-CD3 IgRs combined with Rituximab (Rtx) or Trastuzumab (Trz) or Rituximab and Trastuzumab when co-cultured with CD20+ Raji B cells and Jurkat-NFAT-Luc T cells in Example 24.

FIG. 44. Evaluation of T cell co-stimulation for anti-CD28 IgRs combined with Rituximab (Rtx) or Trastuzumab (Trz) when co-cultured with CD20+ Raji B cells and Jurkat-NFAT-Luc T cells in wells pre-coated with 1 μg/mL OKT3 in Example 24.

FIG. 45. Evaluation of T cell co-stimulation for anti-CD137 IgRs combined with Rituximab (Rtx) or Trastuzumab (Trz) or Rituximab and Trastuzumab when co-cultured with CD20+ Raji B cells and HEK-Luc-CD137 cells in Example 24.

FIG. 46. Evaluation of T cell co-stimulation for anti-CD89 IgRs combined with OKT3 or Trastuzumab (Trz) or OKT3 and Trastuzumab when co-cultured with Jurkat-NFAT-Luc T cells and CHO-K1-CD89 cells in Example 24.

FIG. 47. Display of SDS-PAGE gels images for Reduced (R) and Non-reduced (NR) IgRs from Example 25 and Marker (M) in kDa.

FIG. 48A and FIG. 48B display of chromatograms from size exclusion chromatography using a high pressure liquid chromatography system (HPLC-SEC) for IgRs from Example 25.

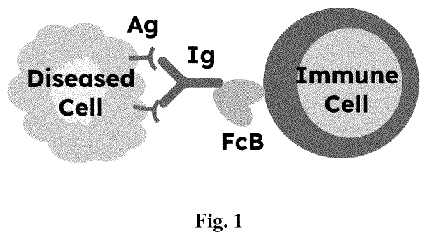

### DETAILED DESCRIPTION OF THE INVENTION

The field of the invention generally relates to immunology, and more specifically relates to heterologous polypeptides or multimeric proteins, and nucleic acids encoding the same, comprising at least one immunoglobulin-binding domain and at least immune cell surface polypeptide or protein binding domain for use in modulating disease such as cancer, autoimmunity, organ rejection, pathogenic infections such as viruses, bacteria, parasites or fungus.

The present invention relates to but is not limited to, heterologous polypeptides or multimeric proteins, comprising an immunoglobulin-binding domain with affinity and specificity for a portion of an immunoglobulin molecule (Ig); and at least one domain with affinity and specificity for at least one extracellular polypeptide present on immune cells.

The present invention relates to heterologous polypeptides or multimeric proteins and nucleic acids encoding the same that comprises at least one immunoglobulin-binding domain and at least one immune cell surface protein-binding domain thus creating an Immunoglobulin Redirector (IgR) molecules capable of engaging immune cells and modulating cellular activity such as effector function or suppression. IgRs of the invention can be used as a monotherapy or in combination with one or more standard, current or experimental therapeutics. such as cancer, immune disorders, or pathogenic infections.

Antibody-based or antibody-fragment based immunotherapies such as plasma derived intravenous immunoglobulin (IVIG), polyclonal antibodies, monoclonal antibodies, antibody fusion proteins, single chain variable fragment (ScFv), a domain antibody (ex a VHH or nanobody) and antibody drug conjugates can treat a wide array of diseases, particularly cancer, inflammatory diseases and infectious diseases. Such therapies may depend on diseased cell elimination by: engagement an Fc-receptor on an effector cell; and recognizing cell surface molecules (for example overexpressed protein on a cancer cell or virus or virus components budding from an infected cell) that is differentially present relative to a normal cell. Binding of an antibody based immunotherapy to a diseased cell can lead to cell death via various mechanisms, for example, antibody dependent cell mediated cytotoxicity (ADCC), antibody dependent cell mediated phagocytosis (ADCP), antibody dependent Neutrophil Extracellular Traps (NETosis), complement dependent cytotoxicity (CDC), or direct cytotoxic activity of the payload from an antibody drug conjugate (ADC). By combining IgRs with antibody based therapies, new cell types and cellular functions can be elicited. Additionally, IgRs can be delivered as monotherapies and rely on a patient's own antibodies to modulate disease.

Immunotherapy including but not limited to cell-based therapies, antibody therapies, cytokines, chemokines, growth factors and receptor or ligand fusions is used to provoke an immune response towards diseased cells, tissues or organs while sparing healthy cells, tissues or organs. Disease areas using immunotherapy include but is not limited to cancer, autoimmunity, inflammation, alloimmunity and infectious disease.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. The term “about” can also encompass variations, which can be up to ±5%, but can also be ±4%, 3%, 2%, 1%, etc. Whether or not modified by the term “about,” the claims include equivalents to the quantities.

As used herein, “domain” (typically a sequence of three or more, generally 5 or 7 or more amino acids, such as 10 to 200 amino acid residues) refers to a portion of a molecule, such as a protein or encoding nucleic acid, that is structurally and/or functionally distinct from other portions of the molecule and is identifiable. For example, domains include those portions of a polypeptide chain that can form an independently folded structure within a protein made up of one or more structural motifs and/or that is recognized by virtue of a functional activity, such as binding activity. A protein can have one, or more than one, distinct domains. For example, a domain can be identified, defined or distinguished by homology of the primary sequence or structure to related family members, such as homology to motifs. In another example, a domain can be distinguished by its function, such as an ability to interact with a biomolecule. A domain independently can exhibit a biological function or activity such that the domain independently or fused to another molecule can perform an activity, such as, for example binding. A domain can be a linear sequence of amino acids or a non-linear sequence of amino acids. Many polypeptides contain a plurality of domains. For exemplification herein, definitions are provided, but particular domains can be recognized in some aspects by name. If needed appropriate software can be employed to identify domains.

An “immunoglobulin heavy chain”, as used herein, is a polypeptide that contains at least a portion of the antigen binding domain of an immunoglobulin and at least a portion of a variable region of an immunoglobulin heavy chain or at least a portion of a constant region of an immunoglobulin heavy chain. Thus, the immunoglobulin derived heavy chain has significant regions of amino acid sequence with a member of the immunoglobulin gene superfamily. For example, the heavy chain in a Fab fragment is an immunoglobulin-derived heavy chain.

An “immunoglobulin light chain”, as used herein, is a polypeptide that contains at least a portion of the antigen binding domain of an immunoglobulin and at least a portion of a variable region of an immunoglobulin light chain or at least a portion of a constant region of an immunoglobulin light chain. Thus, the immunoglobulin derived light chain has significant regions of amino acid sequence with a member of the immunoglobulin gene superfamily.

An “Fc region” or “Fc domain”, as used herein, refers to the crystallizable fragment which is the region of an antibody with interacts with the cell surface receptors (Fc receptors).

An “immunoglobulin molecule”, as used herein, is a protein containing the immunologically-active portions of an immunoglobulin heavy chain and immunoglobulin light chain covalently coupled together and capable of specifically combining with antigen. For example, the immunoglobulin molecules can be IgG, IgE, IgD, IgA, IgM and IgY. For example, the subclasses of the immunoglobulin molecules can be IgG1, IgG2, IgG3, IgG4, IgA1, and IgA2.

In some aspects, the specific dose level for any particular subject or patient may depend upon a variety of factors including the activity of the specific compound employed, the age, body weight, general health, route of administration, severity of the disorder, rate of excretion. The therapeutically effective amount for a given situation can be readily determined by routine experimentation and is within the skill and judgment of the ordinary clinician. The terms “therapeutically effective amount” and “therapeutically effective dose” are used interchangeably herein to refer to an amount of a compound that results in prevention or amelioration of systems in a patient or a desired biological outcome. The terms “subject” and “patient” are used interchangeably herein to refer to a mammal, such as a human, non-human primate (e.g., a baboon, an orangutan, a monkey, a gorilla), or a non-primate mammal (e.g., a mouse, a rat, a dog, a pig).

The immune cell surface protein binding domain can be any suitable molecule that can bind the receptor, such as a small organic molecule, an antigen-binding portion of an antibody (e.g., a Fab fragment, a Fab′ fragment, a F(ab′)2 fragment, a scFv fragment, a Fv fragment, a dsFv diabody, a dAb fragment, a Fd′ fragment, a Fd fragment, or an isolated complementarity determining region (CDR) region), an antibody mimetic (e.g., an aptamer, an affibody, an affilin, an affimer, an anticalin, an avimer, a DARPin, and the like), a nucleic acid, lipid, and the like.

The IgR can comprise any moiety that inhibits the ability of an immunoglobulin to bind and/or activate its receptor. The blocking moiety can inhibit the ability of the immunoglobulin to bind and/or activate its receptor sterically blocking and/or by noncovalently binding to the immunoglobulin. Examples of suitable blocking moieties include the full length or a immunoglobulin-binding fragment or mutein of the cognate receptor of the immunoglobulin. Antibodies and fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like that bind the immunoglobulin can also be used. Other suitable antigen-binding domain that bind the immunoglobulin can also be used, include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of suitable blocking polypeptides include polypeptides that sterically inhibit or block binding of the immunoglobulin to its cognate receptor.

Advantageously, such moieties can also function as half-life extending elements. For example, a peptide that is modified by conjugation to a water-soluble polymer, such as PEG, can sterically inhibit or prevent binding of the immunoglobulin to its receptor. Polypeptides, or fragments thereof, that have long serum half-lives can also be used, such as serum albumin (human serum albumin), immunoglobulin Fc, transferrin and the like, as well as fragments and muteins of such polypeptides.

Antibodies and antigen-binding domains that bind to, for example, a protein with a long serum half-life such as HSA, immunoglobulin or transferrin, or to a receptor that is recycled to the plasma membrane, such as FcRn or transferrin receptor, can also inhibit the immunoglobulin, particularly when bound to their antigen. Examples of such antigen-binding polypeptides include a single chain variable fragment (scFv), single-domain antibody such as a heavy chain variable domain (VH), a light chain variable domain (VL) and a variable domain of camelid-type nanobody (VHH), a dAb and the like. Other suitable antigen-binding domain that bind the immunoglobulin can also be used, include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds.

The engineered scaffold can comprise a sdAb, a scFv, a Fab, a VHH, a fibronectin type III domain, immunoglobulin-like scaffold, DARPin, cystine knot peptide, lipocalin, three-helix bundle scaffold, protein G-related albumin-binding module, or a DNA or RNA aptamer scaffold.

The serum half-life extension element can also be antigen-binding polypeptide that binds to a protein with a long serum half-life such as serum albumin, transferrin and the like. Examples of such polypeptides include antibodies and fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like. Other suitable antigen-binding domain include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of antigen-binding polypeptides include a ligand for a desired receptor, a ligand-binding portion of a receptor, a lectin, and peptides that binds to or associates with one or more target antigens.

The binding moieties can be any kind of polypeptides. For example, in certain instances the binding moieties are natural peptides, synthetic peptides, or fibronectin scaffolds, or engineered bulk serum proteins. The bulk serum protein comprises, for example, albumin, fibrinogen, or a globulin. In some embodiments, the binding moieties are engineered scaffolds. Engineered scaffolds comprise, for example, sdAb, a scFv, a Fab, a VHH, a fibronectin type III domain, immunoglobulin-like scaffold (Halaby et al., 1999), DARPin, cystine knot peptide, lipocalin, three-helix bundle scaffold, protein G-related albumin-binding module, or a DNA or RNA aptamer scaffold.

In some embodiments the heterologous polypeptide or multimeric protein and nucleic acids encoding the same, comprises:
(a) at least one immunoglobulin-binding domain
(b) and at least one immune cell surface protein-binding domain

In some embodiments the heterologous polypeptide or multimeric protein and nucleic acids encoding the same, comprises:
(a) at least one immunoglobulin-binding domain wherein the immunoglobulin binding domain is an antigen-binding domain, antibody or antigen-binding fragment, including but not limited to ScFv, Fab, a single domain antibody, VHH, nanobody, avimer, a Fab, (Fab)2, a Kunitz domain, a small modular immunopharmaceutical (SMIP), an adnectin, an affibody, a DARPin, an anticalin or a synthetic peptide such as Fc-III their derivatives or analogs thereof with affinity and specificity for one or more immunoglobulin isotypes and derivatives thereof, including but not limited to IgG, IgA, IgM, IgE, IgD
(b) and at least one immune cell surface protein-binding domain

In various embodiments, an immunoglobulin-binding domain is an antigen-binding domain, antibody or antigen-binding fragment including derivatives and analogs thereof that is fully human or humanized.

In some embodiments at least one immunoglobulin-binding domain is an antigen-binding domain, antibody or antigen-binding fragment including derivatives and analogs thereof with affinity and specificity for the Fc, Fc-glycan, hinge, constant heavy chain including CH1, CH2, CH3, CH4, constant light chain kappa, constant light chain lambda, variable heavy chain framework region, variable light chain framework region, Fab or (Fab)2 of one or more immunoglobulin isotypes including IgG, IgA, IgM, IgE and IgD.

Immunoglobulin binding antibodies have been previously reported (Hamilton & Morrison, 1993), (Hermans et al., 2015), (Hermans et al., 2017), (Bonvin et al., 2015), (Belin et al., 2004; Carrier et al., 1995) (Leonard et al., 2022) and (Ji Yi H K, 2021).

In some embodiments the immunoglobulin-binding domain is anti-Fc VHH (SEQ ID NO: 48), anti-IgG comprising VH/VL pair 17F12 (SEQ ID NO: 134, SEQ ID NO: 135), anti-CH1 VHH (SEQ ID NO: 130) or the CDRs therein. In other embodiments the immunoglobulin-binding domain is derived from anti-IgG clone HG2-25, 8E11, 8F1, NH3/130.5.2, NH3/15.8, HP6045, MS-278, anti-IgG Fc MK1A6, JDC-10, H2, 6F11C8, AbD27686, R10Z8E9, M1310G05, 97924, PABZ-080, RF-AN, A4, NA6, HP6017 (SEQ ID NO: 288, SEC ID: 290), HP6070, GG-7, Fc-III peptide (Univ, 2010), clone EM-07; anti-IgA clones in (Chang et al., 2017), H15A43, B35064B, A9604D2; anti-IgM clones in (Frey et al., 2018), SA-DA4, M15/8, B481; anti-IgE clones HuMaE11/Omalizumab, TES-C21; anti-IgD clones IGD26, IADB6; anti-lambda clones N10/2, JDC-12; anti-kappa clones NH3/41.34, SB81A, anti-IgG Fab clone 4A11, anti-IgG CH2 clone 8A4.

In some embodiments at least one immunoglobulin-binding domain is an antigen-binding domain, antibody or antigen-binding fragment including derivatives and analogs thereof with affinity and specificity for the Fc region of one or more immunoglobulin isotypes including IgG, IgA, IgM, IgE and IgD and blocks Fc receptor binding to the Fc region of one or more immunoglobulin isotypes including IgG, IgA, IgM, IgE and IgD.

In some embodiments at least one immunoglobulin-binding domain is an antigen-binding domain, antibody or antigen-binding fragment including derivatives and analogs thereof with affinity and specificity for the Fc region of one or more immunoglobulin isotypes including IgG, IgA, IgM, IgE and IgD and does not block Fc receptor binding to the Fc region of one or more immunoglobulin isotypes including IgG, IgA, IgM, IgE and IgD.

Fc receptors are classified based on the isotype of the antibody to which it is able to bind. For example, Fc-gamma receptors (FcγR) generally bind to IgG antibodies, such as one or more subtype thereof (i.e., IgG1, IgG2, IgG3, IgG4); Fc-alpha receptors (FcαR) generally bind to IgA antibodies; Fc-alpha/mu (Fcα/μR) receptors generally bind IgA and IgM; Fc-epsilon receptors (FcεR) generally bind to IgE antibodies; and IgDR receptors generally bind to IgD. Additionally, other Fc receptors have been reported for various isotypes such as Fc neonatal receptor (FcRn) capable of binding IgG (Raghavan et al., 1994); FcRL4 and FcRL5 capable of binding IgG and IgA (Wilson et al., 2012), DC-SIGN capable of binding IgG (Anthony et al., 2008), TRIM21 capable of binding IgG (Keeble et al., 2008), MMR capable of binding IgG glycans (Dong et al., 1999), Dectin-1 and Dectin-2 capable of binding glycans on IgG (Boesch et al., 2014; Karsten et al., 2012) mannose binding lectin 2 (MBL2) capable of binding glycans on Ig (Arnold et al., 2006), C1q capable of binding IgG, IgM and IgA; and pIgR capable of binding IgA and IgM. In some embodiments, the Fc-binder is an Fc receptor comprising an Fc-gamma receptor, an Fc-alpha receptor, an Fc-epsilon receptor, an Fc-alpha/mu receptor. Examples of Fc-gamma receptors include, without limitation, CD64A (FcγRI), CD64B (FcγRI), CD64C (FcγRI), CD32A (FcγRIIA in including H131 and R131 alloytypes), CD32B (FcγRIIB), CD32C (FcγRIIC), CD16A (FcγRIIIA, including V176 and F158 allotypes also referred to the V158 and F158 allotypes excluding leader sequence or L66H mutation (de Vries et al., 1996), and CD16B (FcγRIIIB including SH, NA1, NA2 allotypes). An example of an Fc-alpha receptor is FcαR1 (CD89). Examples of Fc-epsilon receptors include, without limitation, Fc∈RI and Fc∈RII/CD23.

Engagement of Fc receptors on innate and adaptive immune cells and tissue specific cells can mediate various cellular functions via engagement with the Fc region of immunoglobulin (Ig) such as IgG, IgA, IgE, IgM and IgD. Ig-Fc can bind to Fc receptors to varying degrees depending on Isotype, subclass, allotype, glycosylation and via point mutations. IgG can bind activating FcγR such as CD16 (FcγRIIIa or FcγRIIIb), CD32A or CD32C (FcγRIIa or FcγRIIc), CD64 (FcγRI), C1q and mediate cellular functions such as antigen presentation, internalization, superoxide generation, ADCC, phagocytosis, cytokine secretion, NETosis, adhesion induction, respiratory burst, degranulation, complement fixation and apoptosis. IgG can bind inhibitory FcγR such as CD32B (FcγRIIb) and can mediate internalization and downregulation of B cells, mast cells, macrophages and NK cells (Boesch et al., 2015). IgG binds FcRn in a pH dependent manner and can mediate internalization, IgG transport and phagocytosis. Additional IgG Fc receptors such as complement C1q, FcRL5, pIgR and DC-SIGN have been shown to bind the Fc domain to varying degrees (Boesch et al., 2014). IgA can bind pIgR, FcαRI, Fcα/μR, C1q and can mediate functions such as transcytosis, phagocytosis, ADCC, oxidative burst, cytokine production, downregulation of TGFb, antigen presentation, complement fixation and immune complex trapping (PMID 25700208). IgM can bind pIgR, Fcα/μR, C1q and can mediate functions such as transcytosis, antigen presentation, immune complex trapping and complement fixation. IgE can bind FcεRI and FcεRII and mediate functions such as immune cell modulation, internalization, antigen trapping, antigen presentation, histamine, cytokine production and cytotoxicity (Delespesse et al., 1989; Shin & Greer, 2015). IgD can bind IgDR and can mediate basophil stimulation, release of immune activating, proinflammatory and antimicrobial mediators (Chen & Cerutti, 2011). Selection of FcγR type and variants by allotype, amino acid mutants, domain swap fusions, modified glycosylation sites and glycoforms can modify affinity and IgG subclass specificity as previously shown (“The Second and Third Extracellular Domains of FcγRI (CD64) Confer the Unique High Affinity Binding of IgG2a,” 1998)(Hulett & Hogarth, 1998; “The Second and Third Extracellular Domains of FcγRI (CD64) Confer the Unique High Affinity Binding of IgG2a,” 1998)(Oganesyan et al., 2015)(Website, n.d.-a)(Shibata-Koyama et al., 2009). Possible FcγR variants with enhanced affinity or modified IgG subclass specificity are exhibited in Example 10.

The immunoglobulin binding domain of the heterologous polypeptides and multimeric proteins described herein includes Fc-binders or polypeptides capable of binding the Fc portion of an immunoglobulin (Ig) molecule (example, IgG, IgA, IgM, IgE or IgD), Suitable Fc-binders may be derived from naturally occurring proteins such as mammalian Fc receptors, certain bacterial proteins such as Protein A or Protein G and virus proteins such as TspB (Müller et al., 2013), gE (Para et al., 1980), gI (Dubin et al., 1990), gpI (Litwin et al., 1992), FcγR-like HCV core protein (Namboodiri et al., 2007), gp34, gp68, gpRL13, gp95, fcr-1 (Corrales-Aguilar et al., 2014). Additionally, Fc-binders may be synthetic polypeptides engineered specifically to bind the Fc portion of any of the Ig molecules described herein with high affinity and specificity.

In some embodiments, the Fc-binder is an extracellular ligand-binding domain of a mammalian Fc receptor. As used herein, an “Fc receptor” is a cell surface bound receptor that is expressed on the surface of many tissue and immune cells (including endothelial cells, epithelial cells, Langerhans, B cells, dendritic cells, natural killer (NK) cells, macrophage, monocytes, myeloid progenitor cells, platelets, neutrophils, mast cells, polymorphonuclear leukocytes, syncytiotrophoblasts, basophils and eosinophils) and exhibits binding specificity to the Fc domain of an antibody. Fc receptors are typically comprised of immunoglobulin (Ig)-like domains with binding specificity to an Fc (fragment crystallizable) portion of an antibody. In some instances, binding of an Fc receptor to an Fc portion of the antibody may mediate effector functions such as antibody dependent cell-mediated cytotoxicity (ADCC).

The Fc receptor used for constructing a heterologous polypeptide or multimeric protein as described herein may be a naturally-occurring polymorphism variant (e.g., the CD16A V158 and F158 variants or CD16B NA1, NA2, SH variants), which may have increased or decreased affinity to Fc as compared to a wild-type counterpart. Alternatively, the Fc receptor may be a functional variant of a wild-type counterpart, which carry one or more mutations (e.g., up to 10 amino acid residue substitutions) that alter the binding affinity to the Fc portion of an Ig molecule. In some instances, the mutation may alter the glycosylation pattern of the Fc receptor and thus the binding affinity to Fc. In some embodiments, the point mutation or mutations of an Fc receptor ablates an enzyme cleavage site such as the ADAM17 cleavage site on FcγR3A and FcγR3B as performed in FcγR3AV-S197P, SEQ ID NO: 23, FcγR3AF-S197P SEQ ID NO: 271, FcγR3B-NA1-S197P SEQ ID NO: 272, FcγR3B-NA2-S197P SEQ ID NO: 273, FcγR3B-SH-S197P SEQ ID NO: 274. Optionally, some or all of an enzyme cleavage site such as the ADAM17 cleavage site can be removed but shortening the Fc receptor sequence as performed in FcγR3AV-short SEQ ID NO: 3, FcγR3AF-short SEQ ID NO: 4, FcγR3B-NA1-short, SEQ ID NO: 275, FcγR3B-NA2-short, SEQ ID NO: 276, FcγR3B-SH-short, SEQ ID NO: 277.

In some embodiments the heterologous polypeptide or multimeric protein and nucleic acids encoding the same, comprises:
(a) at least one immunoglobulin-binding domain derived from an Fc receptor including but not limited to FcγRIIIa, FcγRIIIa with amino acids LVGSKNV in domain 2 replaced by MGKHRY, FcγRIV, FcγRIIIb, FcγRIIIb with amino acids LVGSKNV in domain 2 replaced by MGKHRY, FcγRIIa, FcγRIIc, FcγRIIb, FcγRI, FcγRI with domain 3 removed, FcRL5, pIgR, FcαRI, Fcα/μR, FcμR, FcεRI, FcεRII, FcRn or TRIM21 including allotypes, derivatives and analogs thereof
(b) and at least one immune cell surface protein-binding domain

In some embodiments the heterologous polypeptide or multimeric protein and nucleic acids encoding the same, comprises:
(a) at least one immunoglobulin-binding domain derived from an Fc receptor wherein a point mutation or mutations of an Fc receptor may ablate an enzyme cleavage site such as the ADAM17 cleavage site
(b) Optionally, in the Fc receptor, some or all of an enzyme cleavage site such as the ADAM17 cleavage site has been removed
(c) and at least one immune cell surface protein-binding domain

In some embodiments the heterologous polypeptide or multimeric protein and nucleic acids encoding the same, comprises:
(a) at least one immunoglobulin-binding domain is derived from an Fc receptor comprising FcγR3AV-S197P, SEQ ID NO: 23, FcγR3AF-S197P SEQ ID NO: 271, FcγR3B-NA1-S197P SEQ ID NO: 272, FcγR3B-NA2-S197P SEQ ID NO: 273, FcγR3B-SH-S197P SEQ ID NO: 274 including any allotypes, derivatives and analogs thereof.
(b) Optionally, is derived from an Fc receptor comprising FcγR3AV-short SEQ ID NO: 3, FcγR3AF-short SEQ ID NO: 4, FcγR3B-NA1-short, SEQ ID NO: 275, FcγR3B-NA2-short, SEQ ID NO: 276, FcγR3B-SH-short, SEQ ID NO: 277 including any allotypes, derivatives and analogs thereof.
(c) and at least one immune cell surface protein-binding domain

In some embodiments the heterologous polypeptide or multimeric protein and nucleic acids encoding the same, comprises:
(a) at least one immunoglobulin-binding domain derived from an Fc receptor including but not limited to FcγRIIIa V158 SEQ ID NO: 1, FcγRIIIa-short V158 SEQ ID NO: 3, FcγRIIIa F158 SEQ ID NO: 2, FcγRIIIa-short F158 SEQ ID NO: 4, FcγRIV SEQ ID NO: 8, FcγRIIIb NA1 SEQ ID NO: 6, FcγRIIIb NA2 SEQ ID NO: 7, FcγRIIIb SH SEQ ID NO: 5, FcγRIIa H131 SEQ ID NO: 9, FcγRIIa R131 SEQ ID NO: 10, FcγRIIb/c SEQ ID NO: 11, FcγRI SEQ ID NO: 12, FcRL5 SEQ ID NO: 13, pIgR SEQ ID NO: 14, FcαRI SEQ ID NO: 15, Fcα/μR SEQ ID NO: 16, FcμR SEQ ID NO: 17, FcεRI SEQ ID NO: 18, FcεRII SEQ ID NO: 19, FcRn SEQ ID NO: 20 or c1q including any allotypes, derivatives and analogs thereof.
(b) and at least one immune cell surface protein-binding domain

In some embodiments at least one immunoglobulin-binding domain derived from a bacterial or virus Fc receptor including but not limited to Protein A, Protein G or ProteinA/G, TspB, gE, gI, gpI, FcγR-like HCV core protein, gp34, gp68, gpRL13, gp95, fcr-1 including derivatives and analogs thereof.

Antibody-based immune checkpoint pathway and co-stimulation modulators have begun to provide new immunotherapeutic approaches for treating cancer (for example checkpoint inhibition by binding PD1, PDL1, CTLA4, OX40, TIM3, LAG3, TIGIT, CD47; co-stimulation by binding CD137, CD28, CD2, CD7; and inflammatory diseases by suppressing immune cell activation by binding SIRP1α, CD11a, CD18, CD80, CD86, and PD1.

Multispecific agents that bind tumor associated antigens or tumor associated antigen peptide-MHC complexes and can redirect to surface proteins on lymphocytes such as T cells, NK cells or Myeloid cells such as macrophages, monocytes or neutrophils and have been gaining traction clinically by targeting effector function mediating receptors including but not limited to the T cell receptor (TCR) complex, including CD3, CD3ε, CD3δ, CD3γ, TCR, TCRα, TCRβ, TCRγ, TCRδ or combinations thereof, CD8, CD4, CD2, Fas Ligand, CD40, CD40L, CD137, CD28, CD56, NKG2D, NKp46, PD1, PDL1, CTLA4, CLEC5A, CD79, BCR, OX40, TIM3, TIGIT, CD7, LAG3, CD11a, CD18, CD80, CD86 dectin-1, dectin-2, dectin-3, FcγRIIIa, FcγRIV, FcγRIIIb, FcγRIIa, FcγRIIc, FcγRIIb, FcγRI, C1q, FcRL5, pIgR, FcαRI, Fcα/μR, FcμR, FcεRI or FcεRII.

In some embodiments or in combination with previous embodiments, the heterologous polypeptide or multimeric protein and nucleic acids encoding the same, comprises:
(a) at least one immunoglobulin-binding domain
(b) and at least one immune cell surface protein-binding domain derived from a natural soluble ligand derivatives or analogs thereof or the extracellular portion of a natural receptor or ligand found on an immune cell including but not limited to Lymphoid cells, Myeloid cells, T cells, B cells, NK cells, Macrophages, Monocytes, NK-T cells, Neutrophils, Dendritic cells, Basophils, Eosinophils and Mast cells

In some embodiments or in combination with previous embodiments, the heterologous polypeptide or multimeric protein comprises:
(a) at least one immunoglobulin-binding domain
(a) at least one immune cell surface protein-binding domain is derived from a natural soluble ligand including but not limited to cytokines, chemokines, pentraxins, galectin-9, HMGB1, TGF-beta, growth factors, pattern recognition proteins, lectins and enzymes, including derivatives or analogs thereof or the extracellular portion of a natural receptor or ligand including but not limited to PD1, PDL1, PDL2, CTLA4, OX40, OX40L, TIM3, TIM1, LAG3, TIGIT, CD137, CD137L, CD28, CD2, CD7, CD11a, CD11b, CD18, CD80, CD86, MHC-I, MHC-II, MHC-G, HLA-DR, CD209, CD206, Galectin-3, LSECtin, FGL1, CD112, CD155, HVEM, CEACAM-1, Fas ligand, TCR complex, TCRα, TCRβ, TCRγ, TCRδ, CD3, CD3ε, CD3γ, CD3δ, CD226, CD27, CD47, SIRP1α, CCR8, TNFR2, CD103, CD39, TIGIT, CD96, VISTA, BTLA, B7-H3, CD8, CD4, dectin-1, dectin-2, dectin-3, chemokine receptors, cytokine receptors, growth factor receptors, pattern recognition receptors, NKG2D, NKp46, MICA, ULBP-1, ULBP-2, BCR, CD79, CD40, CD40L and enzymes including derivatives or analogs thereof.

In some embodiments or in combination with previous embodiments, a heterologous polypeptide or multimeric protein comprises
(a) at least one immunoglobulin-binding domain
(b) At least one immune cell surface protein-binding domain is an antigen-binding domain, antibody or antigen-binding fragment including derivatives or analogs thereof with affinity and specificity for the extracellular portion of a natural receptor or ligand including but not limited to PD1, PDL1, PDL2, CTLA4, OX40, OX40L, TIM3, TIM1, LAG3, TIGIT, CD137, CD137L, CD28, CD2, CD7, CD11a, CD11b, CD18, CD80, CD86, MHC-I, MHC-II, MHC-G, HLA-DR, CD209, CD206, Galectin-3, LSECtin, FGL1, CD112, CD155, HVEM, CEACAM-1, Fas ligand, TCR complex, TCRα, TCRβ, TCRγ, TCRδ, CD3, CD3ε, CD3γ, CD3δ, CD226, CD27, CD47, SIRP1α, CCR8, TNFR2, CD103, CD39, TIGIT, CD96, VISTA, BTLA, B7-H3, CD8, CD4, chemokine receptors, cytokine receptors, growth factor receptors, pattern recognition receptors, NKG2D, NKp46, MICA, ULBP-1, ULBP-2, BCR, CD79, CD40, CD40L, FcγRIIIa, FcγRIV, FcγRIIIb, FcγRIIa, FcγRIIc, FcγRIIb, FcγRI, FcRL5, pIgR, FcαRI, Fcα/μR, FcμR, FcεRI, FcεRII, DC-SIGN, CLEC5A dectin-1, dectin-2, dectin-3,
In some embodiments or in combination with previous embodiments, a heterologous polypeptide or multimeric protein comprises
(a) at least one immunoglobulin-binding domain
(b) at least one immune cell surface protein-binding domain is an antigen-binding domain, antibody or antigen-binding fragment including derivatives or analogs thereof with affinity and specificity for the extracellular portion of a natural receptor or ligand including but not limited to Lymphoid cells, Myeloid cells, T cells, B cells, NK cells, Macrophages, Monocytes, NK-T cells, Neutrophils, Dendritic cells, Basophils, Eosinophils and Mast cells

In some aspects, it is desirable to have a heterologous polypeptide or multimeric protein with a long half-life in vivo. Multiple half-life extension strategies have been previously described (Strohl, 2015) including but not limited to proteins, polypeptides, protein fusions and like comprising: human serum albumin (HSA), anti-HSA antibody, transferrin, anti-transferrin antibody, Fc, PEGylation, XTENylation, PASylation, HAPylation, ELPylation, GLP fusion, CTP fusion. Monomer Fc fusion proteins have been previously described (Wang et al., 2017) which can enable longer half-life and some Fc receptor binding while maintaining a smaller molecular weight.

In some aspects, it is desirable to increase the half-life of heterologous polypeptide or multimeric protein comprising an Fc polypeptide where the Fc region has amino acid mutation or mutations that can further enhance the half-life of the molecule in vivo. Multiple Fc-mutational strategies have been previously described (Saunders, 2019) that enable half-life enhancement including but not limited to amino acid mutation or mutations: R435H, N434A, M252Y/S254T/T256E, M428L/N434S, T252L/T253S/T254F, E294(deletion)/T307P/N434Y, T256N/A378V/S383N/N434Y or glycosylation engineering for example E294(deletion) inducing increased Fc-glycan sialylation which in turn increases half-life.

In some aspects, it is desirable to have an Fc domain that does not substantially bind an immunoglobulin-binding domain such as an Fc receptor or antibody. Multiple strategies have been previously described that enable substantially reduced Fc receptor binding (Saunders, 2019; Strohl, 2015) in an Fc region including but not limited amino acid mutation or mutations: L235E, L234A/L235A, S228P/L235E in IgG4, L234A/L235A/P239G, P331S/L234E/L235F, N265A, G237A, E318A, E233P, G236R/E328R, IgG2/IgG4 cross-subclass, H268Q/V209L/A330S/P331S, V234A/G237A/P238S/H268A/A330S/P331S, A330L, N270A, K322A, P329A, P331A, IgG2/IgG3 cross-subclass, V264A, F241A, N297A, N297G, N297Q, S228P/F234A/L235A or N297 glycosylation engineering, modification or removal including but not limited to high mannose or enzymatic deglycosylation or trimming.

In some aspects, the IgR is a multimeric protein comprising an immunoglobulin binding domain that is an anti-CH1 antibody fragment and an human IgG CH1 domain with mutation F122Y (Kabat numbering) to ablate the IgR's ability to bind itself, “self-bind” or “self-associate.”

In some aspects, the IgR is a multimeric protein comprising an immunoglobulin binding domain that is an anti-IgG antibody fragment comprising clone 17F12 CDRs and some or all of a human constant heavy chain domain with one or multiple amino acid mutations including but not limited to CH1-P13S; CH1-K87E; hinge: IgG1-EPKSCDKTH, hinge IgG2-ERKCCV, hinge IgG3-ELKTPLGDTTH, IgG4-ESKYGP to IgG-EFTP; CH2-E64R; CH3-T10I; CH3-S14P; CH3-K30T; CH3-T711; CH3-V92T to ablate the IgR's ability to “self-bind.”

In some aspects or in combination with previous embodiments, a heterologous polypeptide or multimeric protein comprises:
(a) at least one immunoglobulin-binding domain
(b) and at least one immune cell surface protein-binding domain
(c) and at least one half-life extension domain including but not limited to human serum albumin (HSA), anti-HSA, transferrin, anti-transferrin, PEGylation, XTENylation, PASylation, HAPylation, ELPylation, GLP fusion, CTP fusion or an Fc polypeptide derived from an immunoglobulin wherein the Fc does not substantially bind said Fc-binding region.

In some embodiments or in combination with previous embodiments a heterologous polypeptide or multimeric protein comprises:
(a) at least one immunoglobulin-binding domain
(b) and at least one immune cell surface protein-binding domain
(c) and at least one half-life extension domain including but not limited to an Fc polypeptide derived from an immunoglobulin wherein the Fc does not substantially bind said immunoglobulin-binding domain by selection of IgG heavy chain Fc polypeptide including the mutations L234A, L235A and P329A or alternatively P329G.

In some embodiments or in combination with previous embodiments a heterologous polypeptide or multimeric protein comprises:
(a) at least one immunoglobulin-binding domain
(b) and at least one immune cell surface protein-binding domain
(c) and at least one half-life extension domain including but not limited to an Fc polypeptide derived from an immunoglobulin wherein the Fc does not substantially bind said immunoglobulin-binding domain and the Fc polypeptide includes mutations M252Y/S254T/T256E.

In some aspects or in combination with previous aspects or embodiments, it is desirable to use amino acid mutation or mutations in the constant regions of antibodies to produce multispecific heterologous polypeptides or hetero-multimeric polypeptides with at least one immunoglobulin-binding domain and at least one immune cell surface protein-binding domain. Multiple protein engineering strategies have been previously described (H. Liu et al., 2017) to produce multispecific proteins including but not limited to Quadromas, Knobs-in-holes cognate light chains, Knobs-in-holes cognate light chains, CrossMab Fab, CrossMab VH-VL, CrossMab CH1-CL, TriMab, OAscFab-IgG, dsFv-IgG, DuetMab, cFae-IgG1, Charged-Pair-ScFv-Fc, SEEDbody, Two-Arm LUZ-Y, kappa-lambda-body, bite, diabody, Tamdab, DART, BiKE, TriKE, mFc-VH or Fcab. Previously described protein engineering strategies include introducing amino acid mutation or mutation in the Fc region to produce hetero-multimeric proteins including but not limited mutations in the Fc CH3-1 and CH3-2 respectively comprising: T366Y and Y407T; S354C/T366W and Y349C, T366S, L368A, Y407V; S364H/F405A and Y349T/T394F; T350V/L351Y/F405A/Y407V and T350V/T366L/K392L/T394W; K392D/K409D and E356K/D399K; IgG1 D221E/P228E/L368E and IgG1 D221R/P228R/K409R; IgG2 C223E/P22SE/L368E and IgG2 C223R/E225R/P228R/K409R; K360E/K409W and Q347R/D399V/F405T; K360E/K409W/Y349C and Q347R/D399V/F405T/S354C; 366K (+351K) and 351D or E or D at 349, 368, 349, or 349+355; Duobody F405L and K409R, SEEDbody IgG/A chimera, BEAT residues from TCRα interface and residues from TCRβ interface; K360D/D399M/Y407A and E345R/Q347R/T366V/K409V; or Y349S/K370Y/T366M/K409V and E356G/E357D/S364Q/Y407A. (Brinkmann & Kontermann, 2017; H. Liu et al., 2017).

In some embodiments or in combination with previous embodiments a multimeric protein comprises:
(a) at least one immunoglobulin-binding domain
(b) and at least one immune cell surface protein-binding domain
(c) and two half-life extension domains including but not limited to an Fc polypeptide derived from an immunoglobulin wherein the Fc does not substantially bind said Fc-binding region
(d) wherein the first and second Fc polypeptides comprise a hetero-multimerization domain wherein the hetero-multimerization domain may be a knob into hole mutation or mutations, leucine zippers, electrostatic, and the like.

In some embodiments or in combination with previous embodiments a multimeric protein comprises:
(a) at least one immunoglobulin-binding domain
(b) and at least one immune cell surface protein-binding domain
(c) and two half-life extension domains including but not limited to an Fc polypeptide derived from an immunoglobulin wherein the Fc does not substantially bind said Fc-binding region by selection of IgG heavy chain Fc polypeptide including the mutations L234A, L235A and P329A or alternatively P329G
(d) wherein the first and second Fc polypeptides comprise a hetero-multimerization domain wherein the hetero-multimerization domain may be a knob into hole mutation, leucine zippers, electrostatic, and the like.

In some embodiments or in combination with previous embodiments, a multimeric protein comprises
(a) at least one immunoglobulin-binding domain
(b) and at least one immune cell surface protein-binding domain
(c) and two half-life extension domains including but not limited to an Fc polypeptide derived from an immunoglobulin wherein the Fe does not substantially bind said Fc-binding region by selection of IgG heavy chain Fc polypeptide including the mutations L234A, L235A and P329A or alternatively P329G
(d) wherein the first Fc polypeptide includes mutation T366W and optionally S354C wherein the second Fc polypeptide includes T366S, L368A and Y407V and optionally Y349C.

In some embodiments or in combination with previous embodiments, a multimeric protein comprises:
(a) at least one immunoglobulin-binding domain
(b) and at least one immune cell surface protein-binding domain
(c) and two half-life extension domains including but not limited to an Fc polypeptide derived from an immunoglobulin wherein the Fc does not substantially bind said Fc-binding region by selection of IgG heavy chain Fc polypeptide including the mutations L234A, L235A and P329A or alternatively P329G and the Fc polypeptide includes mutations M252Y/S254T/T256E
(d) wherein the first Fc polypeptide includes mutation T366W and optionally S354C wherein the second Fc polypeptide includes T366S, L368A and Y407V and optionally Y349C.

In some embodiments or in combination with previous embodiments, it is desirable to produce a multimeric protein with a Fab scaffold that has amino acid mutations introduced that remove the native interchain disulfide bond formation between the constant heavy chain and constant light chain and in some instances introduce a buried interchain disulfide between the constant heavy chain and constant light chain as previously described (Brinkmann et al., 1993; Geddie et al., 2022)(Nakamura et al., 2018), within one of the chains (Hagihara & Saerens, 2014; Nakamura et al., 2018) or as a solution to improve light-chain pairing within multi-specific antibodies (Vaks et al., 2018). (Geddie et al., 2022)

In some embodiments or in combination with previous embodiments, a multimeric protein comprises:
(a) at least one immunoglobulin-binding domain
(b) and at least one immune cell surface protein-binding domain
(c) and at least one immunoglobulin kappa or lambda light chain, derivatives and analogs thereof
(d) and at least one CH1 domain derivatives and analogs thereof
(e) Optionally, the constant heavy chain domain 1 contains mutation C233S or the like and constant light chain C214S or the like to prevent interchain disulfide bond formation
(f) Optionally, the constant heavy chain domain 1 contains mutation C233S and F174C or the like and constant light chain C214S and S176C or the like to prevent interchain disulfide bond formation and to create a buried interchain disulfide bond

In some aspects or in combination with other embodiments or aspects, it is desirable to have multiple immunoglobulin-binding domains to alter the biological activity or improve the overall affinity and avidity of a heterologous polypeptides or multimeric proteins that comprise at least one immune cell surface protein-binding domain. Strategies to produce immunoglobulin-binding domains comprising multiple FcγR in a head-to-tail arrangement or multi-meric Fc-fusions have been previously described or contemplated (Hogarth & Wines, 2014; Johnson et al., 2010; Rueger et al., 2018).

In some embodiments or in combination with previous embodiments, a heterologous polypeptide or multimeric protein comprises:
(a) a first immunoglobulin-binding domain
(b) and at least one immune cell surface protein-binding domain
(c) and a second immunoglobulin-binding domain wherein the second immunoglobulin-binding domain is different from the first immunoglobulin domain
(d) optionally the second immunoglobulin-binding domain is the same from the first immunoglobulin-binding domain wherein the domains are not arranged head to tail if they are derived from FcγR type Fc receptors

In some aspects or in combination with other embodiments or aspects, it is desirable to have multiple immune cell surface protein-binding domains to alter the biological activity or improve the overall affinity and avidity of a heterologous polypeptides or multimeric proteins that comprise at least one immunoglobulin binding domain. For instance it has been previously described that engaging CD3 and co-stimulation or immune checkpoint targets can provide benefit to T cell engagers, for instance CD3 and CD28 (Promsote et al., 2023), CD3 and CD137 (L. Liu et al., 2019) CD3 and PD1 (Herrmann et al., 2018).

In some embodiments or in combination with previous embodiments, a heterologous polypeptide or multimeric protein comprises:
(a) at least one immunoglobulin-binding domain
(b) and a first immune cell surface protein-binding domain
(c) and a second immune cell surface protein-binding domain
(d) Optionally, a second immune cell surface protein-binding domain wherein the second immune cell surface protein binding domain is the same as the first

In some aspects, cysteines are used in antibody fragments described herein to enable site-specific conjugation to a lipid or polymer. Antibodies modified by engineering cysteines into specific sites for conjugation have been previously described (Pillow et al., 2014)(Junutula et al., 2008)(Beck et al., 2017)(Jeffrey et al., 2013)(Tumey et al., 2017). Optionally, non-natural amino acids can be incorporated into specific sites for conjugation (Hallam et al., 2015).

In some embodiments or in combination with previous embodiments, a heterologous polypeptide or multimeric protein comprises:
(a) at least one immunoglobulin-binding domain
(b) and at least one immune cell surface protein-binding domain
(c) and a free cysteine at or near the C-terminus or optionally at or near the N-terminus.

In some embodiments or in combination with previous embodiments, a heterologous polypeptide or multimeric protein comprises:
(a) at least one immunoglobulin-binding domain
(b) and at least one immune cell surface protein-binding domain
(c) and a covalently linked lipid component.

In some embodiments or in combination with previous embodiments, a heterologous polypeptide or multimeric protein comprises:
(a) at least one immunoglobulin-binding domain
(b) and at least one immune cell surface protein-binding domain
(c) and a covalently linked lipid, PEG-lipid or optionally cholesterol component.

In some embodiments or in combination of previous embodiments, the present invention contemplates a method using a heterologous polypeptide or multimeric protein to treat disease as a monotherapy or in combination with one or more standard, current or experimental therapeutics to treat cancer, immune disorders and pathogenic infections including but not limited to: Rituximab, Trastuzumab, Cetuximab, Bevacizumab, Ofatumumab, Pertuzumab, Obinutuzumab, Racotumomab, Ramucirumab, Alemtuzumab, Necitumumab, Dinutuximab, Daratumumab, Elotuzumab, Olaratumab, Atezolizumab, Inotuzumab ozogamicin, Avelumab, Durvalumab, Gemtuzumab ozogamicin, Mogamulizumab (mogamulizumab-kpkc), Cemiplimab (cemiplimab-rwlc), Emapalumab (emapalumab-lzsg), Moxetumomab pasudotox (moxetumomab pasudotox-tdfk), Polatuzumab vedotin (polatuzumab vedotin-piiq), Enfortumab vedotin (enfortumab vedotin-ejfv), [fam-]trastuzumab deruxtecan, (fam-trastuzumab deruxtecan-nxki), Isatuximab (isatuximab-irfc), Sacituzumab govitecan (sacituzumab govitecan-hziy), Tafasitamab (tafasitamab-cxix), Belantamab mafodotin (belantamab mafodotin-blmf), Naxitamab-gqgk, Margetuximab-cmkb, Loncastuximab tesirine, Dostarlimab, Amivantamab, Tisotumab vedotin, tisotumab vedotin-tftv, Relatlimab, Tremelimumab, Nivolumab, Pembrolizumab, ipilimumab, Sotrovimab, Casirivimab, imdevimab, Regdanvimab, Relatlimab, Tixagevimab, cilgavimab, Nirsevimab, Mirvetuximab soravtansine.

In some embodiments or in combination of previous embodiments, the present invention contemplates a kit comprising heterologous polypeptide or multimeric protein.

As stated herein, the pharmaceutical compositions comprise one or more linker sequences. A linker sequence serves to provide flexibility between polypeptides, such that, for example, the immunoglobulin binding domain and the immune cell surface protein binding domain are capable of engaging their targets simultaneously. The linker sequence can be located between any or all of the immunoglobulin binding domains, immune cell surface binding domains, the serum half-life extension element, and/or the blocking moiety. Suitable linkers can be of different lengths, such as from 1 amino acid (e.g., Gly) to 20 amino acids, from 2 amino acids to 15 amino acids, from 3 amino acids to 12 amino acids, including 4 amino acids to 10 amino acids, amino acids to 9 amino acids, 6 amino acids to 8 amino acids, or 7 amino acids to 8 amino acids, and may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. A blocking moiety can prevent one or more immunoglobulin binding domains or one or more immune cell surface protein binding domains or both from substantially engaging their targets. The linker can comprise a cleavage site specific to environmental conditions including but not limited to pH, redox potential, electrical potential and enzyme such as metalloproteases. The linker can be designed to be conditionally broken or change conformation to relieve the blocking moiety from preventing the immunoglobulin binding domains or the immune cell surface protein binding domains from substantially engaging their targets.

he method can further involve the administration of one or more additional agents to treat cancer, such as chemotherapeutic agents (e.g., Adriamycin, Cerubidine, Bleomycin, Alkeran, Velban, Oncovin, Fluorouracil, Thiotepa, Methotrexate, Bisantrene, Noantrone, Thiguanine, Cytaribine, Procarabizine), immuno-oncology agents (e.g., anti-PD-L1, anti-CTLA4, anti-PD-1, anti-CD47, anti-GD2, anti-SIRP1α), cellular therapies (e.g., CAR-T, T-cell therapy), oncolytic viruses and the like.

In some embodiments, the heterologous polypeptide comprises: (a) one or more Fc-binders with affinity and specificity for the Fc portion of an immunoglobulin molecule (Ig); and (b) at least one domain, including derivatives and analogs thereof, with affinity and specificity for at least one extracellular polypeptide, present on immune cells where the Fc-binder comprises the extracellular domain of FcγRI, FcγRIIA, FcγRIIB/C, FcγRIIIA, FcγRIIIB, FcαRI, Fcα/μR, FcεRI, FcεRII, pIgR or FcRn including alloytpes, derivatives and analogs thereof. Selection of the ligand binding domain of an Fc receptor for use in the heterologous polypeptide described her, ein will be apparent to one of skill in the art. For example, it may depend on factors such as the isotype of the antibody to which binding of the Fc receptor is desired and the desired affinity of the binding interaction.

In some embodiments, the heterologous polypeptide comprises: (a) one or more Fc-binders with affinity and specificity for the Fc portion of an immunoglobulin molecule (Ig); and (b) at least one domain, including derivatives and analogs thereof, with affinity and specificity for at least one extracellular polypeptide, present on immune cells where the Fc-binder may be synthetic polypeptides engineered specifically to bind the Fc portion of any of the Ig molecules described herein with high affinity and specificity. For example, such an Fc-binder can be an antibody or an antigen-binding fragment thereof that specifically binds the Fc portion of an immunoglobulin. Examples include, but are not limited to, fragment antigen-binding region (Fab) or (Fab)2, a single-chain variable fragment (scFv), a domain antibody, or a nanobody. Alternatively, an Fc-binder can be a synthetic peptide that specifically binds the Fc portion, such as, but not limited to, a Kunitz domain, a small modular immunopharmaceutical (SMIP), an adnectin, an avimer, an affibody, a DARPin, or an anticalin, Fc-III peptide (Multiple resolution-JaLC, n.d.) which may be identified by screening a peptide combinatory library for binding activities to Fc.

In some embodiments, the heterologous polypeptide comprises: (a) one or more Fc-binders with affinity and specificity for the Fc portion of an immunoglobulin molecule (Ig); and (b) at least one domain, including derivatives and analogs thereof, with affinity and specificity for at least one extracellular polypeptide, present on immune cells where the Fc-binder may be derived from naturally occurring proteins such as mammalian Fc receptors, certain bacterial proteins such as Protein A or Protein G and virus proteins such as TspB, gE, gI, gpI, FcγR-like HCV core protein, gp34, gp68, gpRL13, gp95, fcr-1.

In some embodiments, the heterologous polypeptide comprises: (a) one or more Fc-binders with affinity and specificity for the Fc portion of an immunoglobulin molecule (Ig); and (b) at least one domain, including derivatives and analogs thereof, with affinity and specificity for at least one extracellular polypeptide, present on immune cells where an extracellular polypeptide (b) comprises the extracellular domain(s) of FcγRI, FcγRII, FcγRIII, FcαRI, Fcα/μR, FcεRI, FcεRII, pIgR, FcRn, T cell receptor (TCR) complex (including CD3R, CD3δ, CD3γ, TCRα, TCRβ, TCRγ, TCRδ or combinations thereof), CD8, CD2, Fas Ligand, CD40L, CD16, CD32, CD64, NKG2D, NKp46, PD1, PDL1, CTLA4, OX40, TIM3, LAG3, TIGIT, CD137, CD28, CD28, CD7, CD11a, CD18, CD80, CD86, CD121a, CD121b, IL-18Rα, IL-18Rβ, CD25, CD122, CD132, CD124, CD213a13, CD127, CD360, CD19, CD20, CD5, IL-9R, CD213a1, CD213a2, IL-15Ra, CD123, CDw131, CDw125, CD131, CD116, CDw131, CD126, CD130, IL-11Ra, CD130, CD114, CD212, LIFR, CD130, OSMR, CDw210, IL-20Rα, IL-20Rβ, IL-14R, CD4, CDw217, CD118, CDw119, LTβR, CD120a, CD120b, CD137L, BCMA, TACI, CD27, CD30, CD95 (Fas), GITR, GITRL, CLEC5A, LTbR, HVEM, TRAILR1-4, Apo3, RANK, OPG, TGF-βR1, TGF-βR2, TGF-βR3, EpoR, TpoR, Flt-3, CD117, CD115, or CDw136, CD47, SIPR1α, CCR8, TNFR2, CD103, CD39, CD79, dectin-1, dectin-2, dectin-3.

In some embodiments, the heterologous polypeptide comprises: (a) one or more immunoglobulin-binding domains with affinity and specificity for a region or multiple regions of an immunoglobulin molecule (Ig); and (b) at least one domain, including derivatives and analogs thereof, with affinity and specificity for at least one extracellular polypeptide, present on immune cells; (c) wherein the heterologous polypeptide additionally is fused to a transmembrane domain and optionally one or more intracellular domains.

In some embodiments, the heterologous polypeptide comprises: (a) one or more immunoglobulin-binding domains with affinity and specificity for a region or multiple regions of an immunoglobulin molecule (Ig); and (b) at least one domain, including derivatives and analogs thereof, with affinity and specificity for at least one extracellular polypeptide, present on immune cells; (c) wherein the heterologous polypeptide is conjugated to a chemical compound or compounds such as a lipid, PEG, cytotoxic drug agent.

Various embodiments are as follows.

1. A heterologous polypeptide comprising:
at least one immunoglobulin binding domain; and
at least one immune cell surface protein-binding domain;
wherein the at least one immunoglobulin binding domain is derived from an Fc receptor or Fc binder, including but not limited to, FcγRIII, mFcγRIV, FcγRIIa, FcγRIIb, FcγRIIc, FcγRI, mFcγRIII, mFcγRIIa, mFcγRIIb, mFcγRI, FcαRI, C1q, FcRL, FcRL5, pIgR, Fcα/μR, FcμR, FcεRI, FcεRII, FcRn, TRIM21, allotypes, derivatives and analogs thereof; wherein the at least one immunoglobulin binding domain is derived from an antigen-binding domain, antibody or antigen-binding fragment, variants, derivatives or analogs thereof comprising VH and VL pairs, ScFv, Fab, IgG, sdAb-VL, sdAb-VH, VHH or avimer, derivatives or analogs thereof,
wherein the heterologous polypeptide comprises one or more half-life extension domains comprising anti-HSA antigen-binding domain, antibody or antigen-binding fragment, variants, derivatives or analogs thereof comprising VHH or single domain antibodies, immunoglobulin IgG Fc domains their variants, derivatives or analogs thereof,
wherein the heterologous polypeptide comprises at least two immunoglobulin binding domains; and
wherein the heterologous polypeptide comprises at least two immune cell surface protein-binding domains.

2. A heterologous polypeptide comprising:
at least one immunoglobulin binding domain; and
at least one immune cell surface protein-binding domain;
wherein the at least one immunoglobulin binding domain comprises all or a portion of the Fc receptor of SEQ ID NO: 1, 2, 3, 4, 5, 6, 7, 8, 9, 10, 11, 12, 13, 14, 15, 16, 17, 18, 19, 20, 23, 77, 80, 271, 272, 273, 274, 275, 276, 277, 294, 296, 298, 300, 324 and 325;
wherein the at least one immunoglobulin binding domain is derived from FcγRIIa comprising one or more mutations comprising R56H, K118N, T120V, L160Q and V172E of SEQ ID NO: 9; or
wherein the at least one immunoglobulin binding domain is derived from FcγRIII comprising one or more mutations comprising S181P, K122N, T124V, Q176E, 190R, T118K, A119L and Y134F of SEQ ID NO: 1;
wherein the heterologous polypeptide comprises at least two immunoglobulin binding domains; and
wherein the heterologous polypeptide comprises at least two immune cell surface protein-binding domains.

3. A heterologous polypeptide comprising:
at least one immunoglobulin binding domain; and
at least one immune cell surface protein-binding domain;
wherein the at least one immunoglobulin binding domain is derived from an antigen-binding domain, antibody or antigen-binding fragment their variants, derivatives or analogs thereof, including but not limited to, VH and VL pairs, ScFv, Fab, IgG, sdAb-VL, sdAb-VH, VHH or avimer, their derivatives or analogs thereof;
wherein the at least one immunoglobulin binding domain comprises at least one of the CDR or FR regions in SEQ ID NO: 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 601, 602, 603, 604, 605, 606, 607, 608, 609, 610, 611, 612, 613, 614, 615, 616, 617, 618, 619, 620, 621, 622, 623, 624, 625, 626, 627, 628, 629, 6230, 631, 632, 633, 634, 635, 636, 637, 638, 639, 640, 641, 642, 643, 644, 645, 646, 647, 648, 649, 650, 651, 652, 653, 654, 655, 656, 657, or the avimer defined in SEQ ID NO: 132;
wherein the heterologous polypeptide comprises at least two immunoglobulin binding domains; and
wherein the heterologous polypeptide comprises at least two immune cell surface protein-binding domains.

4. A heterologous polypeptide comprising:
at least one immunoglobulin binding domain;
at least one immune cell surface protein-binding domain;
wherein at least one immune cell surface protein-binding domain is derived from an antigen-binding domain, antibody or antigen-binding fragment their variants, derivatives or analogs thereof, including but not limited to, VH and VL pairs, ScFv, Fab, IgG, sdAb-VL, sdAb-VH, VHH, their derivatives or analogs thereof;
wherein the immune cell surface protein-binding domain comprises one or more CDR or FR regions in SEQ ID NO: 580, 606, 608, 609, 610, 611, 612, 615, 626, 635, 636, 637, 638, 639, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 840, 841, 842, 843, 844, 845, 846, 847, 848, 849, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 940, 941, 942, 943, 944, 945, 946, 947, 948, 949, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046 and 1047;
wherein the heterologous polypeptide comprises at least two immunoglobulin binding domains; and
wherein the heterologous polypeptide comprises at least two immune cell surface protein-binding domains.

5. A heterologous polypeptide comprising:
at least one immunoglobulin binding domain;
at least one immune cell surface protein-binding domain;
at least one half-life extension domain;
wherein the at least one half-life extension domain comprises anti-HSA antigen-binding domain, antibody or antigen-binding fragment their variants, derivatives or analogs thereof comprising VHH or single domain antibodies, comprising one or more CDR or FR regions in SEQ ID NO: 558, 559, 560, 561, 562, 563, 564, 565, 566, 567 and 568;
wherein the heterologous polypeptide comprises at least two immunoglobulin binding domains; and
wherein the heterologous polypeptide comprises at least two immune cell surface protein-binding domains.

6. A heterologous polypeptide comprising:
at least one immunoglobulin binding domain;
at least one immune cell surface protein-binding domain;
at least one half-life extension domain;
wherein the heterologous polypeptide is a single chain of the structure:
D1-D2-D3
and
wherein the at least one half-life extension domain comprises anti-HSA antigen-binding domain, antibody or antigen-binding fragment their variants, derivatives or analogs thereof comprising VHH or single domain antibodies, comprising one or more CDR or FR regions in SEQ ID NO: 558, 559, 560, 561, 562, 563, 564, 565, 566, 567 and 568.

7. The heterologous polypeptide of claim 5, further comprising one or more linkers between the domains.

8. A heterologous polypeptide comprising:
at least two immunoglobulin binding domain;
at least one immune cell surface protein-binding domain;
at least one half-life extension domain;
wherein the heterologous polypeptide is a single chain with at least four domains of the structure:
D1-D2-D3-D4
and
wherein the at least one half-life extension domain comprises anti-HSA antigen-binding domain, antibody or antigen-binding fragment their variants, derivatives or analogs thereof comprising VHH or single domain antibodies, comprising one or more CDR or FR regions in SEQ ID NO: 558, 559, 560, 561, 562, 563, 564, 565, 566, 567 and 568.

9. The heterologous polypeptide of claim 6, further comprising one or more linkers between the domains.

10. A multimeric protein wherein regions of the molecule comprise:
at least one immunoglobulin binding domain;
at least one immune cell surface protein-binding domain; and
two half-life extension domains comprising a first Fc polypeptide and a second Fc polypeptide derived from an immunoglobulin wherein the Fc does not substantially bind the immunoglobulin binding domain;
wherein the first Fc polypeptide and the second Fc polypeptide comprise a hetero-multimerization domain wherein the hetero-multimerization domain is at least one knob into hole mutation;
wherein the multimeric protein comprises at least two immunoglobulin binding domains; and
wherein the multimeric protein comprises at least two immune cell surface protein-binding domains.

11. A multimeric protein wherein regions of the molecule comprise:
at least one immunoglobulin binding domain;
at least one immune cell surface protein-binding domain;
two half-life extension domains comprising a first Fc polypeptide and a second Fc polypeptide derived from an immunoglobulin wherein the Fc does not substantially bind the immunoglobulin binding domain by selection of a IgG heavy chain Fc polypeptide comprising mutations L234A, L235A and P329A or P329G in the constant heavy chain domain 2 (EU Numbering);
wherein the first Fc polypeptide and the second Fc polypeptide comprise a hetero-multimerization domain wherein the hetero-multimerization domain is at least one knob into hole mutation;
wherein the multimeric protein comprises at least two immunoglobulin binding domains; and
wherein the multimeric protein comprises at least two immune cell surface protein-binding domains.

12. A multimeric protein wherein regions of the molecule comprise:
at least one immunoglobulin binding domain;
at least one immune cell surface protein-binding domain;
two half-life extension domains including but not limited to a first Fc polypeptide and a second Fc polypeptide derived from an immunoglobulin wherein the Fc does not substantially bind said immunoglobulin binding domain by selection of a IgG heavy chain Fc polypeptide comprising mutations L234A, L235A and P329A or P329G in the constant heavy chain domain 2 (EU Numbering);
wherein the first Fc polypeptide comprises mutation T366W; and
wherein the second Fc polypeptide comprises T366S, L368A and Y407V in the constant heavy chain domain 3 (EU Numbering);
wherein the first Fc polypeptide further comprises S354C and wherein the second Fc polypeptide further comprises Y349C in the constant heavy chain domain 3 (EU Numbering);
wherein the multimeric protein comprises at least two immunoglobulin binding domains; and
wherein the multimeric protein comprises at least two immune cell surface protein-binding domains.

13. A multimeric protein wherein regions of the molecule comprise:
at least one immunoglobulin binding domain;
at least one immune cell surface protein-binding domain;
at least one immunoglobulin kappa or lambda constant light chain, their variants, derivatives and analogs thereof;
at least one immunoglobulin constant heavy chain domain 1 and at least a portion of the immunoglobulin hinge region, their variants, derivatives and analogs thereof,
wherein the one or more constant heavy chain domain 1 and at least a portion of the hinge comprises mutation C233S and constant light chain C214S (Kabat Numbering);
wherein the one or more constant heavy chain domain 1 and at least a portion of the hinge comprises mutation C233S and F174C and constant light chain C214S and S176C (Kabat Numbering);
wherein at least a first pair of constant heavy chain and at least a portion of the hinge that comprises no mutation at C233 and no mutation in constant light chain at C214 and at least a second pair of constant heavy chain domain 1 at least a portion of the hinge comprises mutation C233S or the like and constant light chain C214S or the like (Kabat Numbering); wherein at least a first pair of constant heavy chain and at least a portion of the hinge that contain no mutation at C233 and no mutation in constant light chain at C214 and at least a second pair of constant heavy chain domain 1 and at least a portion of the hinge comprises mutation C233S and F174C and the constant light chain comprises mutation C214S and S176C (Kabat Numbering);
wherein at least a first pair of constant heavy chain domain 1 and at least a portion of the hinge comprises mutation C233S and constant light chain C214S and at least a second pair of constant heavy chain domain 1 and at least a portion of the contains mutation C233S and F174C and constant light chain C214S and S176C (Kabat Numbering);
wherein the heterologous polypeptide comprises one or more half-life extension domains comprising anti-HSA antigen-binding domain, antibody or antigen-binding fragment, variants, derivatives or analogs thereof comprising VHH or single domain antibodies, immunoglobulin IgG Fc domains their variants, derivatives or analogs thereof,
wherein the multimeric protein comprises at least two immunoglobulin binding domains; and
wherein the multimeric protein comprises at least two immune cell surface protein-binding domains.

14. A multimeric protein wherein a region or regions of the molecule comprise:
at least two immunoglobulin binding domain;
at least one immune cell surface protein-binding domain;
at least a second immunoglobulin-binding domain wherein the second immunoglobulin binding domain is separated from the first immunoglobulin binding domain by a linker from 1 amino acid to 20 amino acids comprising regions of the human constant heavy chain domain 1, kappa chain domain, lambda chain domain, polypeptides comprising linkers 13 amino acids or less, linkers comprising 6 amino acids or less, the constant heavy chain domain 1 derived spacer ASTKGPSVFPLAP, ASTKGP or ASTKGPSVFPLAS, the constant kappa chain derived spacer RTVAAPSVFIFPP or RTVAAP, the constant lambda chain derived spacer SQPKAAPSVTLFP, GQPKANPTVTLFP, GQPKAAPSVTLFP, SQPKAA, GQPKAN or GQPKAA, (GGGS)1, (GGGS)2, (GGGS)3, (GGGS)4;
wherein at least one immunoglobulin binding domain comprises one or more CDR or FR regions in SEQ ID NO: 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 601, 602, 603, 604, 605, 606, 607, 608, 609, 610, 611, 612, 613, 614, 615, 616, 617, 618, 619, 620, 621, 622, 623, 624, 625, 626, 627, 628, 629, 6230, 631, 632, 633, 634, 635, 636, 637, 638, 639, 640, 641, 642, 643, 644, 645, 646, 647, 648, 649, 650, 651, 652, 653, 654, 655, 656, 657, or the avimer defined in SEQ ID NO: 132; and
wherein at least a second immunoglobulin-binding domain wherein the second immunoglobulin-binding domain is the same as the first immunoglobulin-binding domain; or
wherein at least a second immunoglobulin-binding domain wherein the second immunoglobulin-binding domain is different from the first immunoglobulin-binding domain.

15. A multimeric protein wherein at least one region comprises:
at least one immunoglobulin binding domain;
at least one immune cell surface protein binding domain;
one or more amino acid mutations in one or more constant or framework domains of human IgG1, IgG2, IgG3 or IgG4, variants, derivatives and analogs thereof wherein the immunoglobulin binding domain does not substantially bind itself or another region or regions of the molecule;
wherein a mutation of one or more amino acids in the constant heavy chain domain 1 comprising F122Y, P126S and K213E (Kabat Numbering);
wherein a mutation of one or more amino acids in a in the constant heavy chain domain 2 comprising N276K, L309V, L234A, L235A and P329A or P329G (EU Numbering);
wherein a heterologous polypeptide or multimeric protein and nucleic acids encoding the same, comprises at least two immunoglobulin binding domains; and wherein a heterologous polypeptide or multimeric protein and nucleic acids encoding the same, comprises at least two immune cell surface protein binding domains.

16. A multimeric protein wherein regions of the molecule comprise:
at least one immunoglobulin kappa or lambda constant light chain, their variants, derivatives and analogs thereof, and
at least one immunoglobulin constant heavy chain domain 1 and at least a portion of the immunoglobulin hinge region, their variants, derivatives and analogs thereof; and two half-life extension domains comprising a first Fc polypeptide and a second Fc polypeptide derived from an immunoglobulin wherein the first and second Fc polypeptides comprise a hetero-multimerization domain wherein the hetero-multimerization domain is selected from at least one knob into hole mutation;
wherein the first Fc polypeptide comprises T366W and wherein the second Fc polypeptide comprises T366S, L368A and Y407V in the constant heavy chain domain 3 (EU Numbering);
wherein the Fc does not substantially bind one or more of its cognate Fc receptors by selection of IgG heavy chain Fc polypeptide comprising L234A, L235A and P329A in the constant heavy chain domain 2 (EU Numbering);
wherein one or more constant heavy chain domain 1 and all, none or a portion of the hinge comprises mutation C233S and constant light chain C214S (Kabat Numbering);
wherein one or more constant heavy chain domain 1 and at least a portion of the hinge comprises mutation C233S and F174C and constant light chain C214S and S176C (Kabat Numbering);
wherein at least a first pair of constant heavy chain and at least a portion of the hinge that comprises no mutation at C233 and no mutation in constant light chain at C214 and at least a second pair of constant heavy chain domain 1 and at least a portion of the hinge comprises a mutation at C233S and a constant light chain C214S (Kabat Numbering);
wherein at least a first pair of constant heavy chain and at least a portion of the hinge that contain no mutation at C233 and no mutation in constant light chain at C214 and at least a second pair of constant heavy chain domain 1 and at least a portion of the hinge comprises mutations at C233S and F174C and the constant light chain comprises mutation C214S and S176C (Kabat Numbering);
wherein at least a first pair of constant heavy chain domain 1 and at least a portion of the hinge comprises a mutation at C233S and constant light chain C214S and at least a second pair of constant heavy chain domain 1 and at least a portion of the hinge contains mutation C233S and F174C and constant light chain C214S and S176C (Kabat Numbering).

17. The multimeric protein of claim 16
wherein the first Fc polypeptide further comprises S354C and wherein the second Fc polypeptide further comprises Y349C in the constant heavy chain domain 3 (EU Numbering); and
wherein IgG heavy chain Fc polypeptide further comprises P329G in the constant heavy chain domain 2 (EU Numbering).

18. A heterologous polypeptide or multimeric protein of claim 1, 2, 3, 4, 13, 14, or 16 wherein an additional region of the molecule comprises a free cysteine at or near the C-terminus.

19. A heterologous polypeptide or multimeric protein of claim 1, 2, 3, 4, 13, 14, or 16 wherein an additional region of the molecule comprises a covalently linked PEG-lipid.

20. A method of treatment comprising administering the heterologous polypeptide or multimeric protein of claim 1, 2, 3, 4, 5, 6, 7, 8, 9, 10, 11, 12, 13, 14, 16 or 17 to a patient in need thereof to treat disease as a monotherapy.

21. A method of treatment comprising administering the heterologous polypeptide or multimeric protein of claim 1, 2, 3, 4, 5, 6, 7, 8, 9, 10, 11, 12, 13, 14, 16 or 17 to a subject in need thereof to treat cancer, immune disorders, or pathogenic infections in combination with at least one selected from standard, current, or experimental therapeutics.

22. A nucleotide encoding the heterologous polypeptide or multimeric protein of claim 1, 2, 3, 4, 5, 6, 7, 8, 9, 10, 11, 12, 13, 14, 16 or 17.

23. A kit comprising the heterologous polypeptide or multimeric protein of claim 1, 2, 3, 4, 5, 6, 7, 8, 9, 10, 11, 12, 13, 14, 16 or 17.

### EXAMPLES

### Example 1. Expression and Purification of FcγR3A/αCD3 and FcγR3A/αTCR Fusions

Fc-binding heterologous polypeptides were encoded into plasmid DNA expression vectors and transiently expressed as recombinant proteins in chinese hamster ovarian (CHO) cells. The proteins were purified using immobilized metal chromatography by incorporating a GGHHHHHH (SEQ ID NO: 21) tag on the C-terminus or by Protein A if the protein comprised an Fc region.

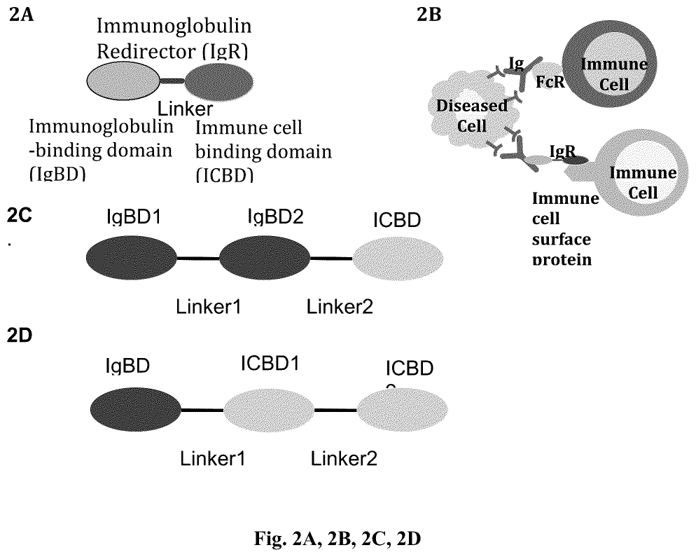

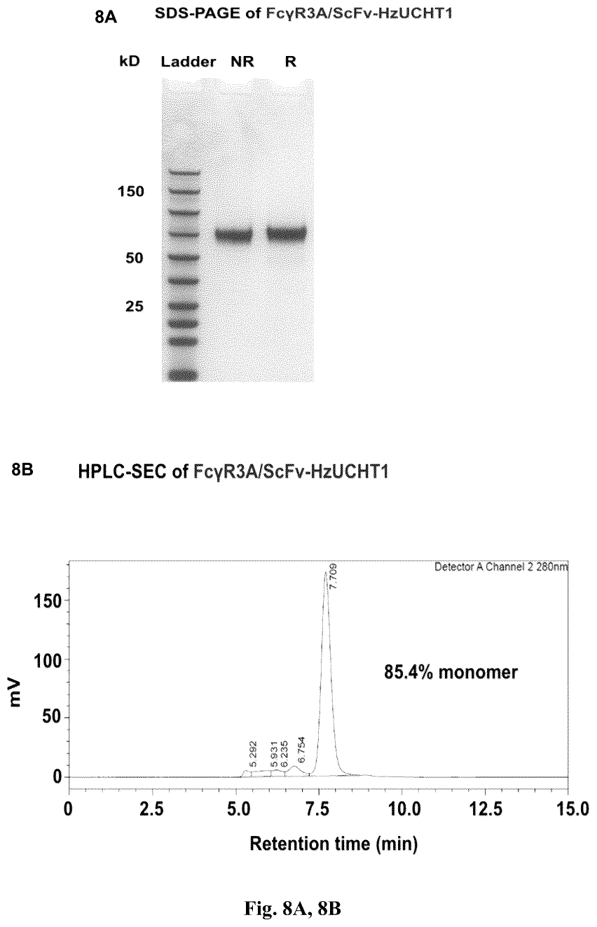

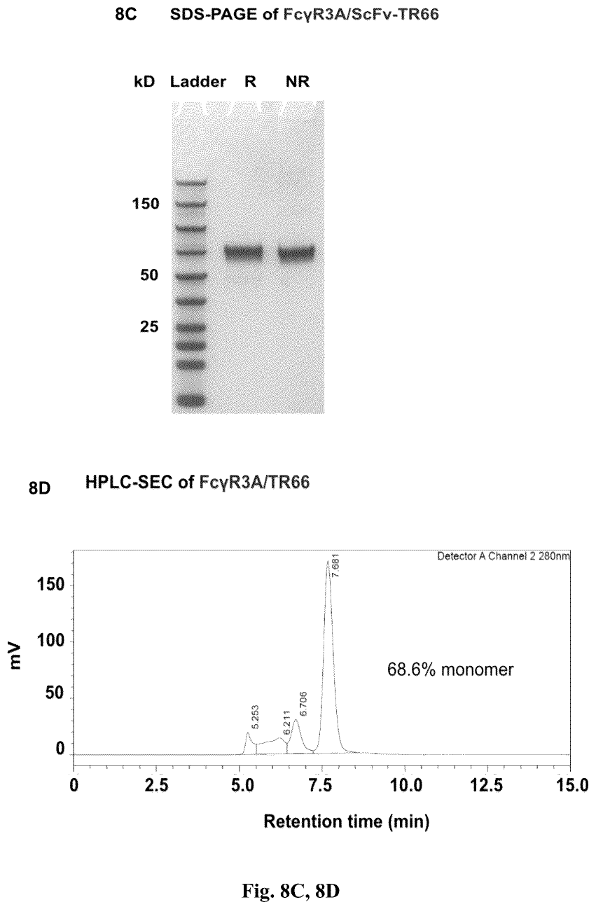

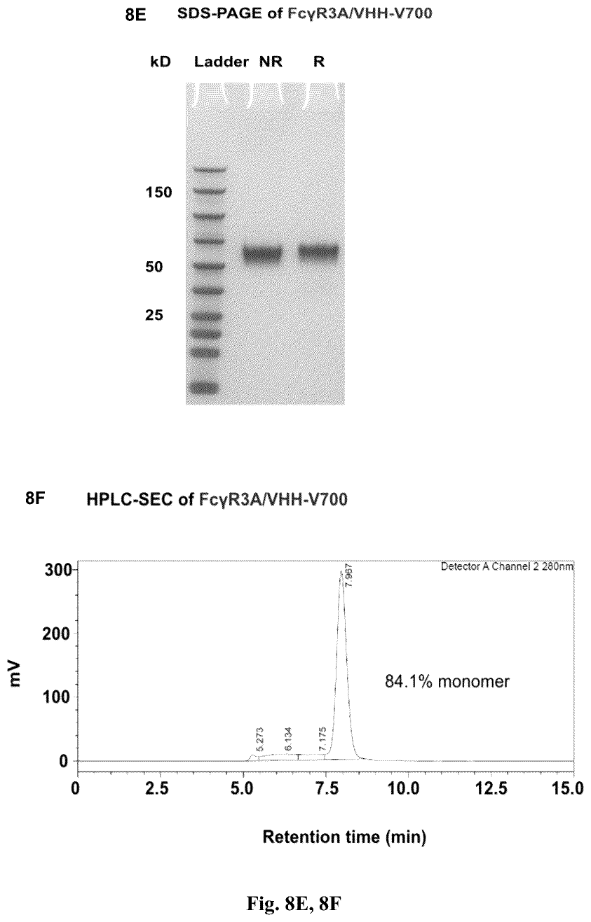

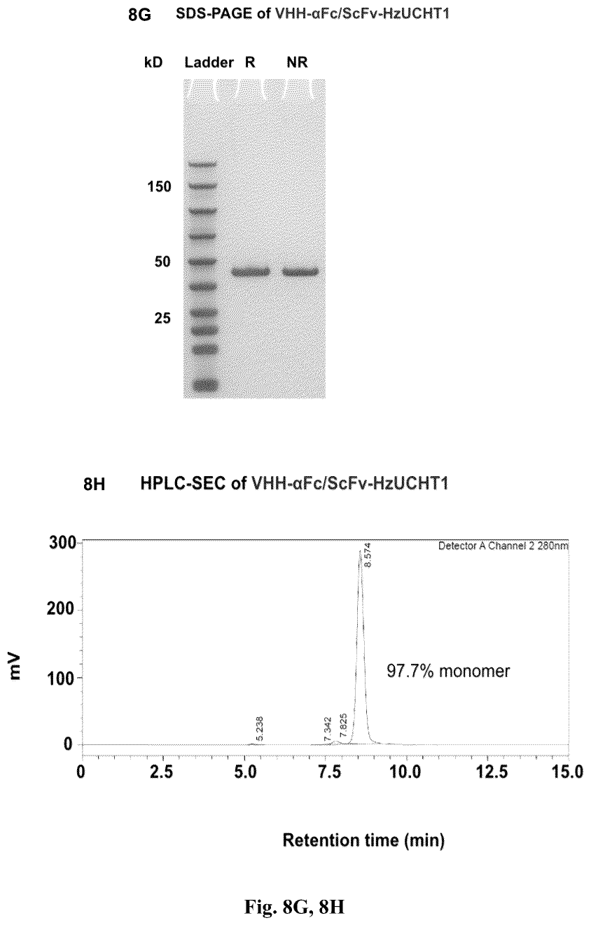

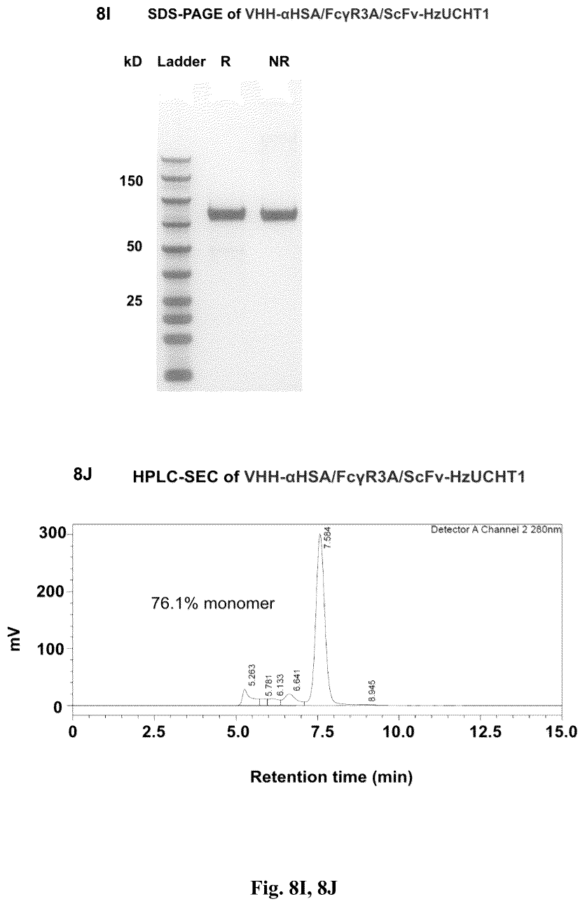

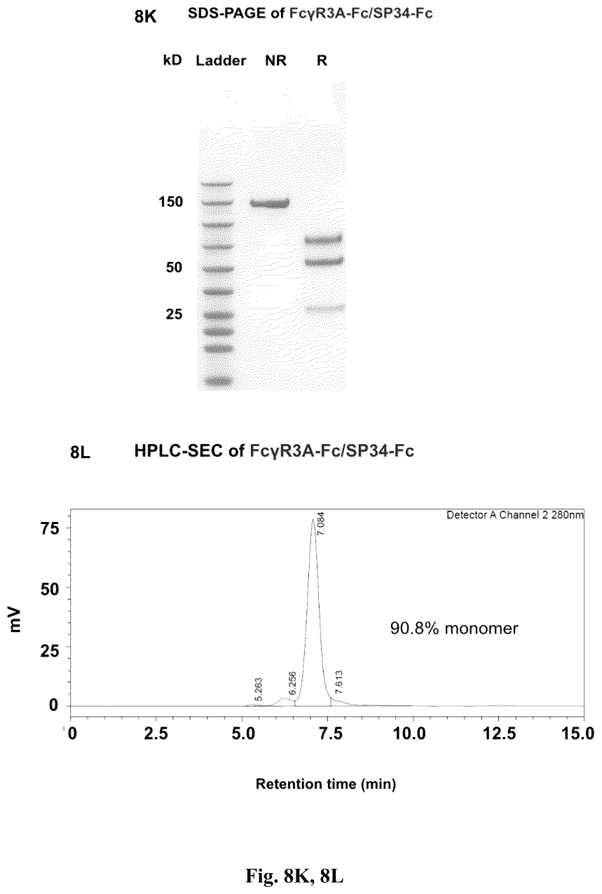

The extracellular domain of the CD16A Fc-receptor (FcγR3A-V158, SEQ ID NO: 1) with an S197P mutation (FcγR3AV-S197P, SEQ ID NO: 23), to ablate susceptibility to ADAM17 cleavage, was fused via a (G4S)₃ linker (SEQ ID NO: 22) to: a humanized version of an αCD3 ScFv clone HzUCHT1 in the VL-VH orientation using a (G4S)₃ linker (VH SEQ ID NO: 24; VL SEQ ID NO: 25, ScFv-HzUCHT1 SEQ ID NO: 26); to form a FcγR3A/ScFv-HzUCHT1 (SEQ ID NO: 27; JIB1, SEQ ID NO: 28) fusion as depicted in FIG. 2A; an αCD3 ScFv clone TR66 in the VH-VL orientation using a (G4S)₃ linker (TR66-VH SEQ ID NO: 29; TR66-VL SEQ ID NO: 30, ScFv-TR66 SEQ ID NO: 31) to form a FcγR3A/ScFv-TR66 fusion (SEQ ID NO: 32; JIB2, SEQ ID NO: 33) as depicted in FIG. 2A; and an αTCR VHH clone V700 (VHH-V700 SEQ ID NO: 34) to form a FcγR3A/VHH-V700 fusion (SEQ ID NO: 35; JIB3, SEQ ID NO: 36) as depicted in FIG. 2A. FIG. 8A, FIG. 8B, FIG. 8C, FIG. 8D, FIG. 8E, FIG. 8F, FIG. 8G, FIG. 8H, FIG. 8I, FIG. 8J, FIG. 8K, and FIG. 8L and Table 1 shows that the proteins were well expressed (by amount recovered using A280 after single step purification) with high purity by both SDS-PAGE and SEC (after single step purification).

TABLE 1
Protein Expression and % Monomer by
SEC for IgRs produced in Example 1.

| Protein | CHO (mg/L) | % Monomer by SEC |
|---|---|---|
| JIB1 | 231 | 85.4% |
| JIB2 | 256 | 68.6% |
| JIB3 | 370 | 84.1% |

### Example 2. Expression and Purification of αHSA/FcγR3A/αCD3 Fusion

Fc-binding heterologous polypeptides were expressed and purified as described in Example 1.

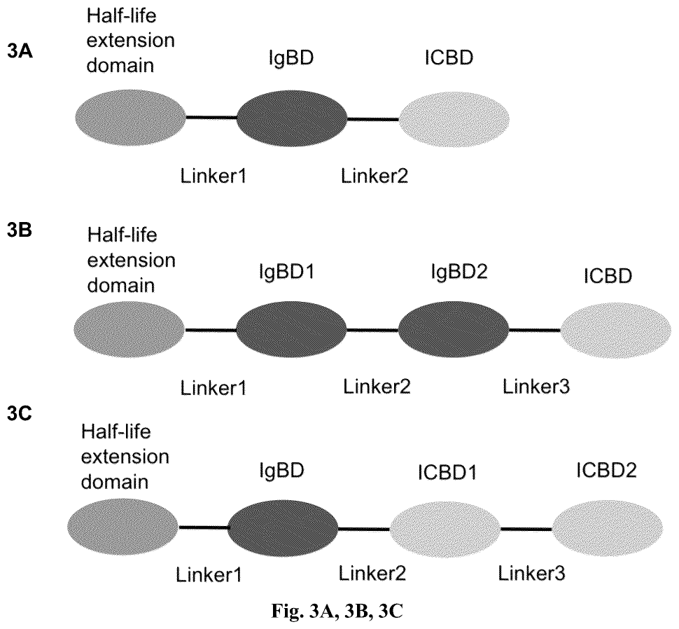

A humanized VHH against HSA (SEQ ID NO: 37) was fused via a (G4S)₃ linker to the extracellular domain of the human CD16A Fc-receptor with an S197P mutation which was fused via a (G4S)₃ linker to a humanized version of an αCD3 ScFv clone HzUCHT1 in the VL-VH orientation using a (G4S)₃ linker to form an αHSA-VHH/FcγR3A/αCD3-ScFv fusion (SEQ ID NO: 38; JIB5, SEQ ID NO: 39) as depicted in FIG. 3A. A 6his-tag with a GG spacer was fused to the C-terminus of the ScFv for purification purposes. FIG. 8A, FIG. 8B, FIG. 8C, FIG. 8D, FIG. 8E, FIG. 8F, FIG. 8G, FIG. 8H, FIG. 8I, FIG. 8J, FIG. 8K, and FIG. 8L and Table 2 show that the proteins were well expressed with high purity by both SDS-PAGE and SEC. Comparing FIG. 8A-FIG. 8B with FIG. 8I-FIG. 8J indicates the addition of the anti-HSA VHH at the N-terminus of FcγRIIIA did not promote a substantial amount of aggregation indicating FcγRIIIA is tolerant of binding regions on both its N and C-terminus.

Table 2. Protein Expression and % Monomer by SEC for IgR produced in Example 2.

TABLE 2
Protein Expression and % Monomer by
SEC for IgR produced in Example 2.

| Protein | CHO (mg/L) | % Monomer by SEC |
|---|---|---|
| JIB5 | 433 | 78.8% |

### Example 3. Expression and Purification of FcγR3A-Fc/αCD3-Fc Heterodimeric Protein

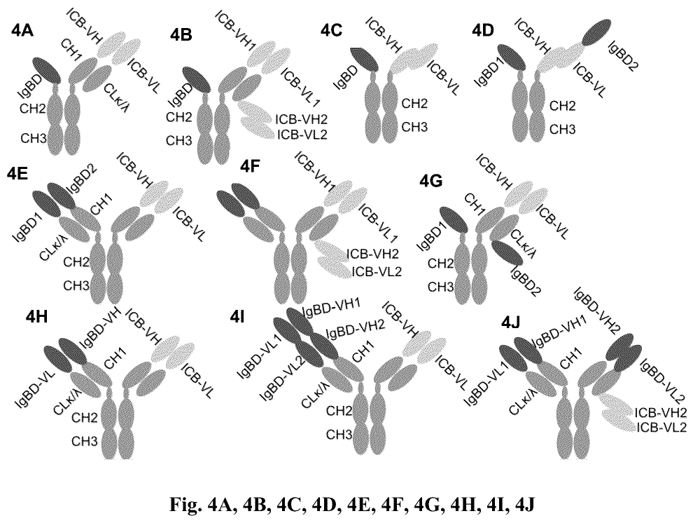

Fc-binding heterodimer protein was expressed and purified as described in Example 1. The FcγR3A-Fc/αCD3-Fc fusion was expressed as 3 polypeptide chains to assemble into one soluble protein comprising two heterodimer forming heavy chains that assembled using knob-in-hole mutations and a light chain that assembles with one of the two heterodimeric heavy chains as depicted in FIG. 4A. One heavy chain comprised of the extracellular domain of the human CD16A Fc-receptor with an S197P mutation was fused via a (G4S)₃ linker to a partial human IgG1 heavy chain with mutations: C233S to prevent disulfide formation; T366S, L368A, Y407V to produce the “hole” Fc heavy chain; and L234A, L235A and P329A (C233S/Fc-LALAPA-hole, SEQ ID NO: 40) to ablate Fc receptor binding; forming an FcγR3A-Fc polypeptide (FcγR3A-S197P/Fc-LALAPA-hole SEQ ID NO: 41). The other heavy chain comprised of a variable heavy chain for the αCD3 clone SP34 (VH-SP34, SEQ ID NO: 42) and a human IgG1 constant heavy chain with mutations: T366W to produce the “knob” Fc heavy chain; and L234A, L235A and P329A (hIgG1-HC/LALAPA-knob, SEQ ID NO: 43) to ablate Fc receptor binding; forming an SP34 human IgG1 heavy chain (SP34-Fc-LALAPA-knob, SEQ ID NO: 44). The light chain comprised of a variable light chain for the αCD3 clone SP34 (SP34-VL, SEQ ID NO: 45) and a human constant lambda light chain (hLC7, SEQ ID NO: 46) forming an SP34 human light chain (SP34-hLC7, SEQ ID NO: 47). FIG. 8A, FIG. 8B, FIG. 8C, FIG. 8D, FIG. 8E, FIG. 8F, FIG. 8G, FIG. 8H, FIG. 8I, FIG. 8J, FIG. 8K, and FIG. 8L and Table 3 show that the proteins were well expressed with high purity by both SDS-PAGE and SEC. FIG. 8K-FIG. 8L indicates the knob-in-hole mutations promoted a near 1:1 ratio of the FcγR3A-Fc and SP34-Fc arms and the FcR KO mutations prevented self-binding between FcγRIIIA and the hetero-dimer Fcs given the high purity.

TABLE 3
Protein Expression and % Monomer by
SEC for IgR produced in Example 3.

| Protein | CHO (mg/L) | % Monomer by SEC |
|---|---|---|
| JIB6 | 107 | 90.8% |

### Example 4. Expression and Purification of αFc/αCD3 Fusion

Fc-binding heterologous polypeptides were expressed and purified as described in Example 1.

An αFc VHH (Fc-10-VHH, SEQ ID NO: 48) capable of binding human Fc was fused via a (G4S)₃ linker to: a humanized version of an αCD3 ScFv clone HzUCHT1 in the VL-VH orientation using a (G4S)₃ linker for form an αFc-VHH/ScFv-HzUCHT1 fusion (SEQ ID NO: 49, SEQ ID NO: 50) as depicted in FIG. 2A. FIG. 8A, FIG. 8B, FIG. 8C, FIG. 8D, FIG. 8E, FIG. 8F, FIG. 8G, FIG. 8H, FIG. 8I, FIG. 8J, FIG. 8K, and FIG. 8L and Table 4 show that the protein was well expressed with high purity by both SDS-PAGE and SEC.

TABLE 4
Protein Expression and % Monomer by
SEC for IgR produced in Example 4.

| Protein | CHO (mg/L) | % Monomer by SEC |
|---|---|---|
| JIB4 | 315 | 97.7% |

### Example 5. Expression and Purification of FcγRIV/αCD3 Fusion

Fc-binding heterologous polypeptides were expressed and purified as described in Example 1.

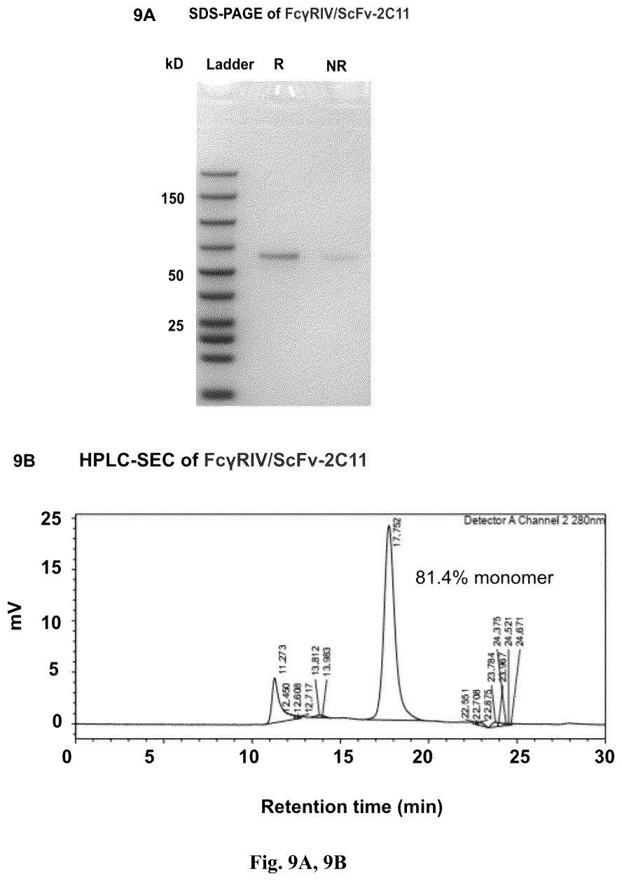

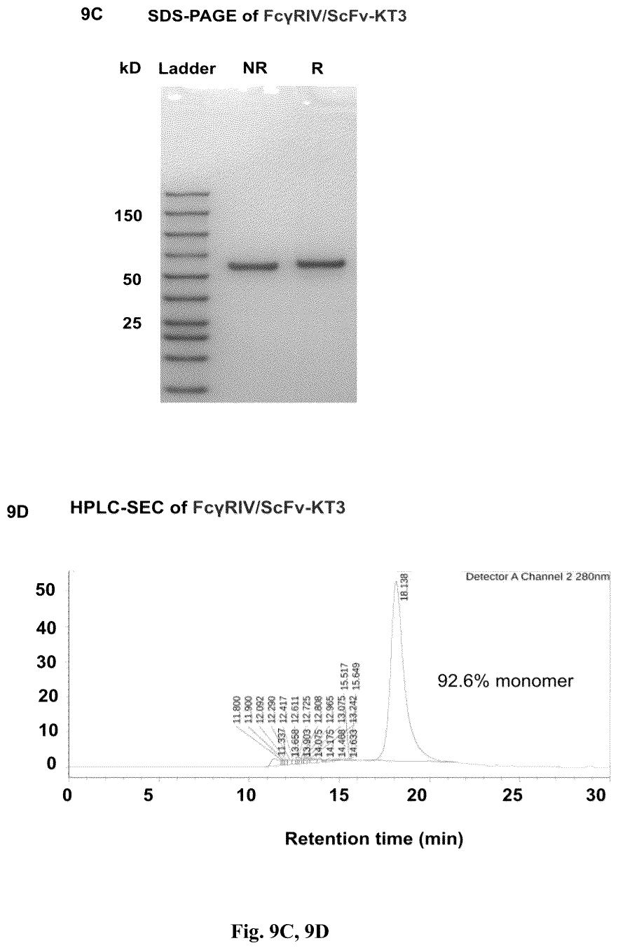

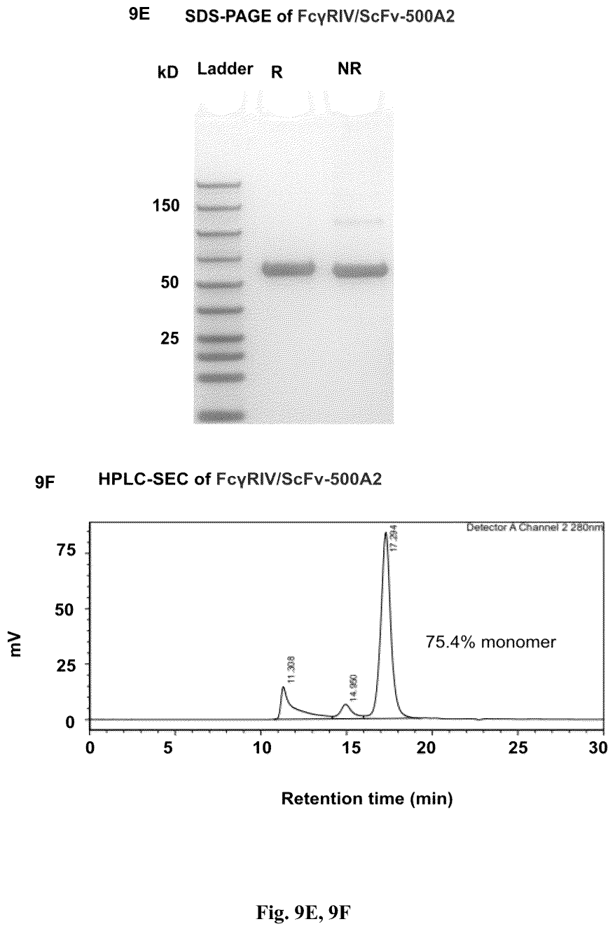

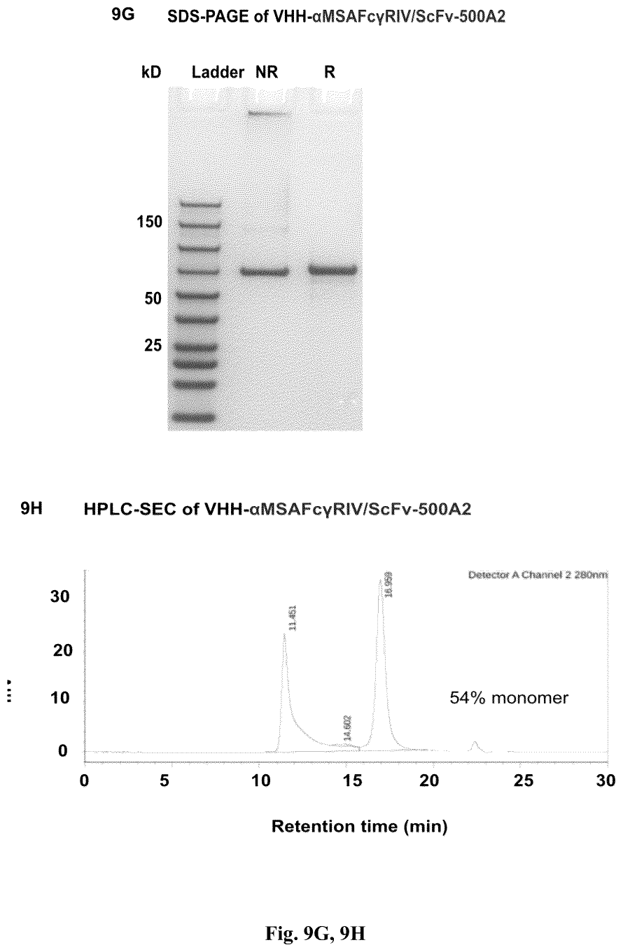

The extracellular domain of the mouse FcγRIV Fc-receptor (mFcγRIV ECD, SEQ ID 8) was fused via a (G4S)₃ linker to: an αCD3 ScFv clone 2C11 in the VH-VL orientation using a (G4S)₄ linker (2C11-VH SEQ ID NO: 51; 2C11-VL SEQ ID NO: 52, ScFv-2C11 SEQ ID 53) for form a FcγRIV/ScFv-2C11 fusion (SEQ ID NO: 54, SEQ ID 55) as depicted in FIG. 2A; an αCD3 ScFv clone KT3 in the VH-VL orientation using a (G4S)₄ linker (KT3-VH SEQ ID NO: 56; KT3-VL SEQ ID NO: 57, ScFv-KT3 SEQ ID 58) to form a FcγRIV/ScFv-KT3 fusion (SEQ ID NO: 59, SEQ 60) as depicted in FIG. 2A; an αCD3 ScFv clone 500A2 in the VH-VL orientation using a (G4S)₄ linker (500A2-VH SEQ ID NO: 61; 500A2-VL SEQ ID NO: 62, ScFv-500A2 SEQ ID NO: 63) for form a FcγRIV/ScFv-500A2 fusion (SEQ ID NO: 64, SEQ ID 65) as depicted in FIG. 2A. FIG. 9A, FIG. 9B, FIG. 9C, FIG. 9D, FIG. 9E, FIG. 9F, FIG. 9G, FIG. 9H, FIG. 9I, and FIG. 9J and Table 5 show that the proteins were well expressed with high purity by both SDS-PAGE and SEC.

TABLE 5
Protein Expression and % Monomer by
SEC for IgR produced in Example 5.

| Protein | CHO (mg/L) | % Monomer by SEC |
|---|---|---|
| FcγRIV/ScFv-2C11-his | 24 | 81.4 |
| FcγRIV/ScFv-KT3-his | 56 | 92.6 |
| FcγRIV/ScFv-500A2-his | 75 | 75.4 |

### Example 6. Expression and Purification of αMSA/FcγRIV/αCD3 Fusion

Fc-binding heterologous polypeptides were expressed and purified as described in Example 1.

A VHH against mouse serum albumin (MSA) (SEQ ID NO: 66) was fused via a (G4S)₃ linker to the extracellular domain of the mouse FcγRIV Fc-receptor (FcγRIV, SEQ ID NO: 24) which was fused via a (G4S)₃ linker to an αCD3 ScFv clone 500A2 in the VH-VL orientation using a (G4S)₄ linker to form an αMSA-VH H/FcγRIV/αCD3-ScFv fusion (SEQ ID NO: 67, SEQ ID NO: 68) as depicted in FIG. 3A. A 6his-tag with a GG spacer was fused to the C-terminus of the ScFv for purification purposes. FIG. 9A, FIG. 9B, FIG. 9C, FIG. 9D, FIG. 9E, FIG. 9F, FIG. 9G, FIG. 9H, FIG. 9I, and FIG. 9J shows that the protein was well expressed with reasonable purity by both SDS-PAGE and SEC from single step purification.

TABLE 6
Protein Expression and % Monomer by
SEC for IgR produced in Example 6.

| Protein | CHO (mg/L) | % Monomer by SEC |
|---|---|---|
| αMSA-VHH/FcγRIV/αCD3- | 48 | 54.0 |
| ScFv-his | | |

### Example 7. Expression and Purification of FcγRIV-Fc/αCD3-Fc Heterodimeric Protein

Fc-binding heterodimeric protein was expressed and purified as described in Example 1.

The FcγRIV-Fc/αCD3-Fc fusion was expressed as 3 polypeptide chains to assemble into one soluble protein comprising two heterodimer forming heavy chains that assembled using knob-in-hole mutations and a light chain that assembles with one of the two heterodimeric heavy chains as depicted in FIG. 4A. One heavy chain comprised of the extracellular domain of the mouse FcγRIV Fc-receptor fused via a (G4S)₃ linker to a partial mouse IgG2a heavy chain with mutations: E356K, T364S, M368L, T370K, D399K and R411T to produce the Knob-in-holes A (KiH-A) Fc heavy chain; and L234A, L235A and P329G (KiH-A/LALAPG/mIgG2a-Fc, SEQ ID NO: 69) to ablate Fc receptor binding; forming an FcγRIV-Fc polypeptide (FcγRIV/mIgG2a-Fc SEQ ID NO: 70). The other heavy chain comprised of a variable heavy chain for the αCD3 clone 500A2 and a human IgG2a constant heavy chain with mutations: T364S, M368L, T370K, K409D, R411T and K439D to produce the Knob-in-holes B (KiH-B) Fc heavy chain; and L234A, L235A and P329G (mIgG2a-HC/KiH-B/LALAPG, SEQ ID NO: 71) to ablate Fc receptor binding; forming an 500A2 human IgG2a heavy chain (500A2-HC-Fc, SEQ ID 72). The light chain comprised of a variable light chain for the αCD3 clone 500A2 (500A2-VL, SEQ ID NO: 32) and a mouse constant kappa light chain (mLC, SEQ ID NO: 73) forming an 500A2 mouse light chain (500A2-mLC, SEQ ID NO: 74). FIG. 9A, FIG. 9B, FIG. 9C, FIG. 9D, FIG. 9E, FIG. 9F, FIG. 9G, FIG. 9H, FIG. 9I, and FIG. 9J and Table 6 show that the protein was well expressed with high purity by both SDS-PAGE and SEC, the knob-in-hole mutations induced approximately 1:1 ratio between FcγRIV-Fc and 500A2-Fc and the FcR-KO Fc mutations prevented any self-binding between FcγRIV and the heterodimer fusion protein Fcs.

TABLE 7
Protein Expression and % Monomer by
SEC for IgR produced in Example 7.

| Protein | CHO (mg/L) | % Monomer by SEC |
|---|---|---|
| SEQ ID 70, 72, 74, | 235 | 96.8 |

### Example 8. Redirecting T Cells to Blood Cancer Cells by Combining Anti-CD20 IgG with IgRs Targeting CD3 or TCR

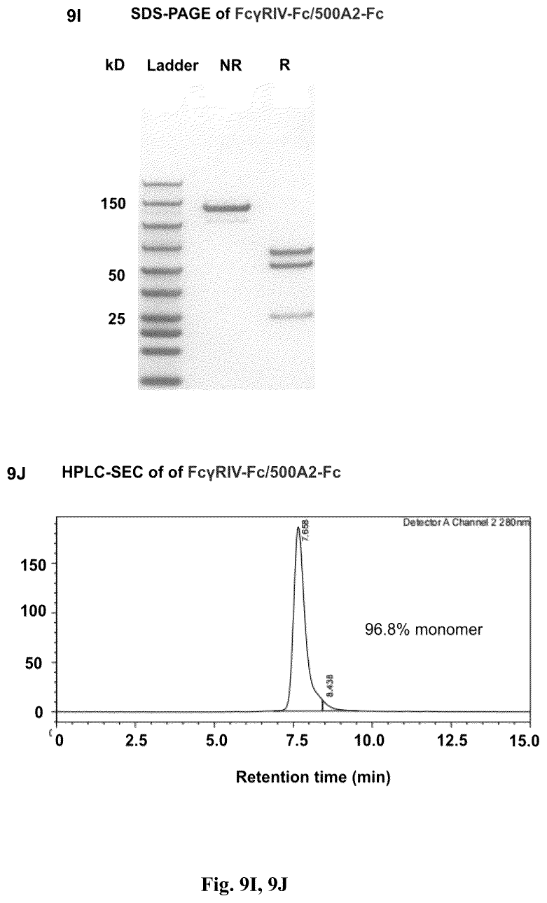

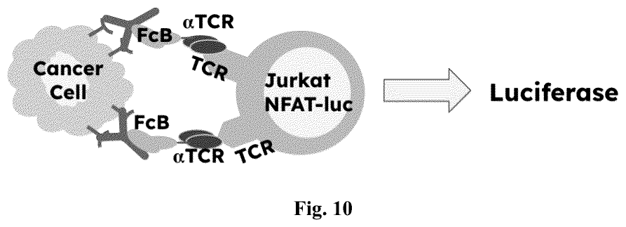

To evaluate the ability of the Fc-binding T cell engaging (FcB-TCE) IgRs to bridge blood cancer cells to T cells a CD20+ Raji cell and CD3/TCR+ Jurkat-CD28-NFAT-Luc co-culture reporter system was employed as displayed in FIG. 10. Commercially available anti-CD20 Rituximab IgG1 (Biointron) was used to opsonize CD20+ Raji cells and bridge to T cells via various IgR proteins including FcγR3A/ScFv-HzUCHT1 fusion (SEQ ID NO: 28); FcγR3A/ScFv-TR66 fusion (SEQ ID NO: 33); FcγR3A/VHH-V700 fusion (SEQ ID NO: 36); αHSA-VHH/FcγR3A/αCD3-ScFv fusion (SEQ ID NO: 39); FcγR3A-Fc/SP34-Fc heterodimer (SEQ ID NO: 41, SEQ ID NO: 44, SEQ ID NO: 47); and an aFc-VHH/ScFv-HzUCHT1 fusion (SEQ ID NO: 50).

The bioassay was performed by (1) collecting Raji and Jurkat-CD28-NFAT1-luc from cell culture flasks and centrifuging at 300× gravity for 5 min; (2) adjusting the density of Raji to 5E5 cells/mL, also adjust Jurkat-CD28-NFAT-luc to 2.5E6 cells/mL; (3) Adding 40 uL Raji and Jurkat-CD28-NFAT-luc respectively into a 96 well U bottom culture plate (NEST) (4) 20 uL Rituximab, Mosunetuzumab or culture medium, RPMI 1640 (Corning)+1% Fetal Bovine Serum (BI), was added into wells, the working concentrations of Rituximab or Mosunetuzumab were 10× the final target concentration or if combining with an IgR 10 ul of Rituximab and 10 ul of IgR was use at 10× the final target concentration; (5) cells and proteins were mixed gently and incubated in the plate at 37° C. for 6 h; (6) after incubation, 100 μl of Bio-Lite luciferase assay reagent (Vazyme) was added and transferred to a ViewPlate-96 White, Clear bottom, TC-treated (Perkin Elmer) and luminescence was measured using a microplate reader.

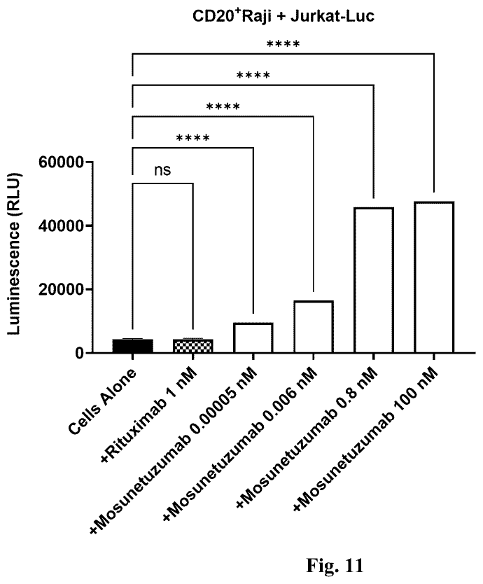

To verify the functionality of the assay system a commercially available anti-CD20/anti-CD3 bispecific, Mosunetuzumab (Biointron), was used as a positive assay control to directly bridge Raji B cells to Jurkat T cells while cells alone or with Rituximab were used as negative controls. FIG. 11 shows that there is no statistical difference between cells alone and cells+Rituximab indicating that Rituximab alone is not capable of bridging B cells to T cells. In contrast, Mosunetuzumab at every concentration evaluated was statistically significantly different from cells alone which is expected given it was specifically designed to bridge CD20+ cells with CD3+ T cells.

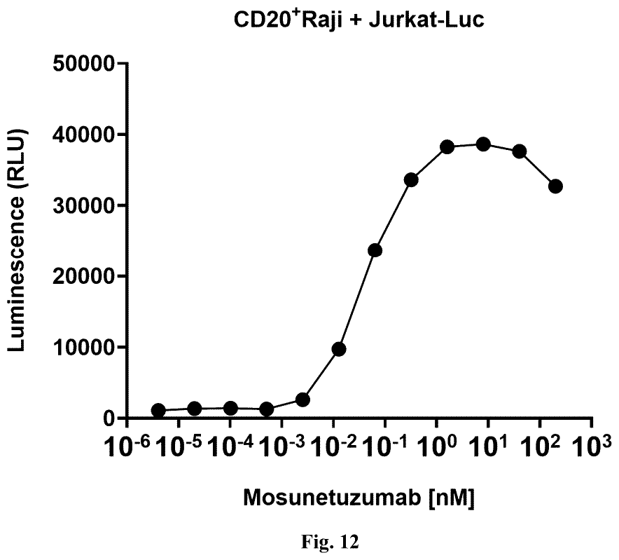

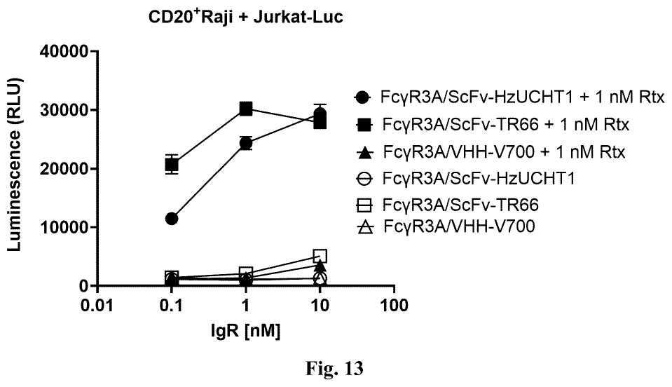

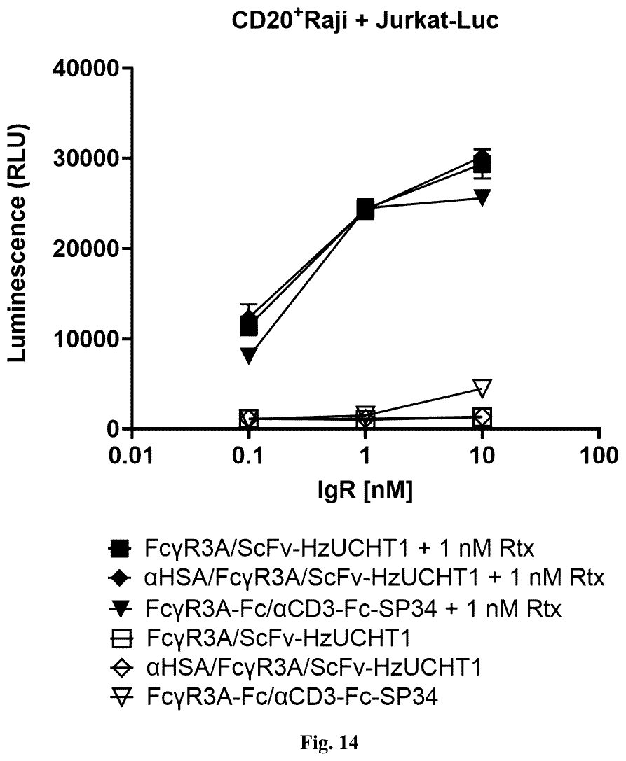

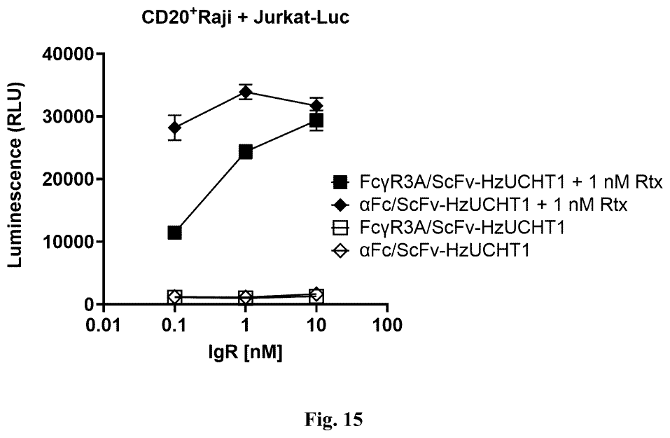

Next, IgRs at 0.1, 1 and 10 nM were evaluated for their ability to bridge CD20+ Raji B cells with T cells and mediate T cell activation with and without the presence of 1 nM Rituximab and with Mosunetuzumab run as a positive control (FIG. 12). As shown in FIG. 13, FIG. 14 and FIG. 15, all 6 designs were capable of driving T cell activation when combined with Rituximab versus without. FIG. 13 shows that the anti-TCR V700 fusion exhibited a lower potency than the anti-CD3 TR66 or anti-CD3 HzUCHT1 fusions and the TR66 fusion exhibited a slightly higher potency than HzUCHT1 indicative of the importance to optimize around the T cell target, the epitope within a T cell target and the affinity against that T cell target. FIG. 14 shows that the IgR designs with half-life extension strategies such as fusion to an anti-HSA VHH or fusion to an IgG Fc with FcR KO mutations show similar potencies indicating that the addition of a VHH at the N-terminal end of the FcγRIIIA region did not interfere with Rituximab IgG-Fc binding and the fusion of an Fe to the C-terminus of the FcγRIIIA region did not interfere with Rituximab IgG-Fc binding. FIG. 15 shows that using an anti-Fc VHH instead of an Fc-receptor such as FcγRIIIA can mediate a higher potency indicating the epitope, affinity and stoichiometry of the Fc-binding domain towards the IgG-Fc are important parameters to optimize (for example FcγR:IgG stoichiometry is 1:1 while anti-Fc VHH:IgG stoichiometry could be 2:1 or 1:1 depending on the epitope). Additionally, most of the IgRs evaluated exhibited maximum luminescence values similar to that of Mosunetuzumab indicating IgRs in combination with target cell specific immunoglobulin are capable of mediating T cell activity similar to that of a traditional T cell engager (TCE).

### Example 9. Redirecting T Cells to Solid Cancer Cells by Combining Anti-HER2 IgG with IgRs Targeting CD3 or TCR

To evaluate the ability of the Fc-binding T cell engaging (FcB-TCE) IgRs to bridge blood cancer cells to T cells a HER2+ SKBR3 cell and CD3/TCR+ Jurkat-CD28-NFAT-Luc co-culture reporter system was employed as displayed in FIG. 10. Commercially available anti-HER2 Trastuzumab IgG1 (Biointron) was used to opsonize HER2+ SKBR3 cells and bridge to T cells via various IgR proteins including FcγR3A/ScFv-HzUCHT1 fusion (SEQ ID NO: 28); FcγR3A/ScFv-TR66 fusion (SEQ ID NO: 33); FcγR3A/VHH-V700 fusion (SEQ ID NO: 36); αHSA-VHH/FcγR3A/αCD3-ScFv fusion (SEQ ID NO: 39); FcγR3A-Fc/SP34-Fc heterodimer (SEQ ID NO: 41, SEQ ID NO: 44, SEQ ID NO: 47); and an aFc-VHH/ScFv-HzUCHT1 fusion (SEQ ID NO: 50).

The bioassay was performed by (1) collecting SKBR3 and culturing overnight at 37C in 100 ul per well in complete medium at 1E5 cells/mL; (2) the next day resuspending with Jurkat-CD28-NFAT-luc at 1E6 cells/mL in 50 ul; (3) 25 uL Rituximab (negative control), Trastuzumab or culture medium, RPMI 1640 (Corning)+1% Fetal Bovine Serum (BI), was added into wells, the working concentrations of Rituximab or Trastuzumab were 4× the final target concentration of 1 nM, 25 ul f IgR was used at 4× the final target concentrations of 0.1, 1 and 10 nM; (5) cells and proteins were mixed gently and incubated in the plate at 37° C. for 6 h; (6) after incubation, 100 μl of Bio-Lite luciferase assay reagent (Vazyme) was added and transferred to a ViewPlate-96 White, Clear bottom, TC-treated (Perkin Elmer) and luminescence was measured using a microplate reader.

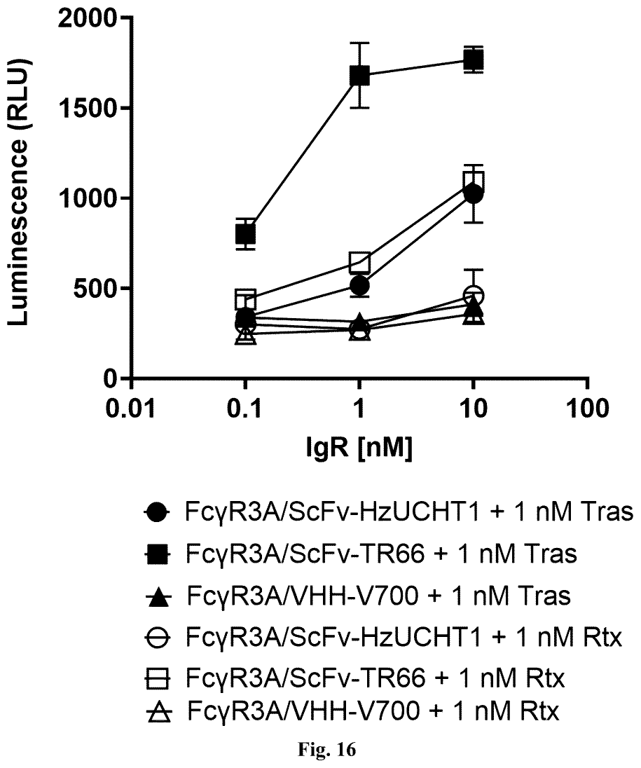

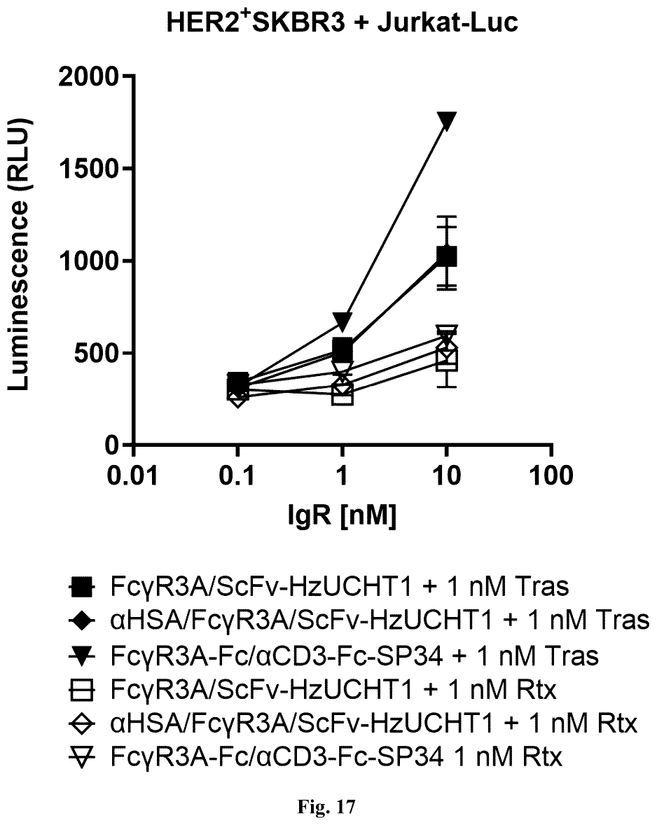

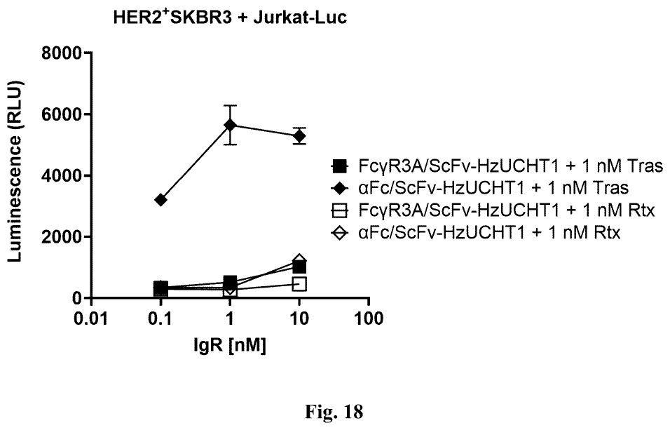

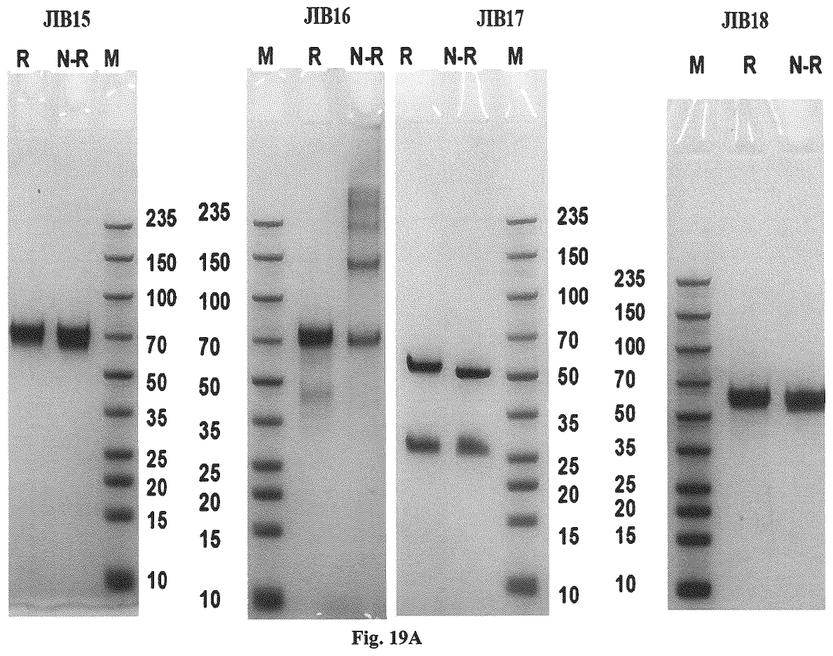

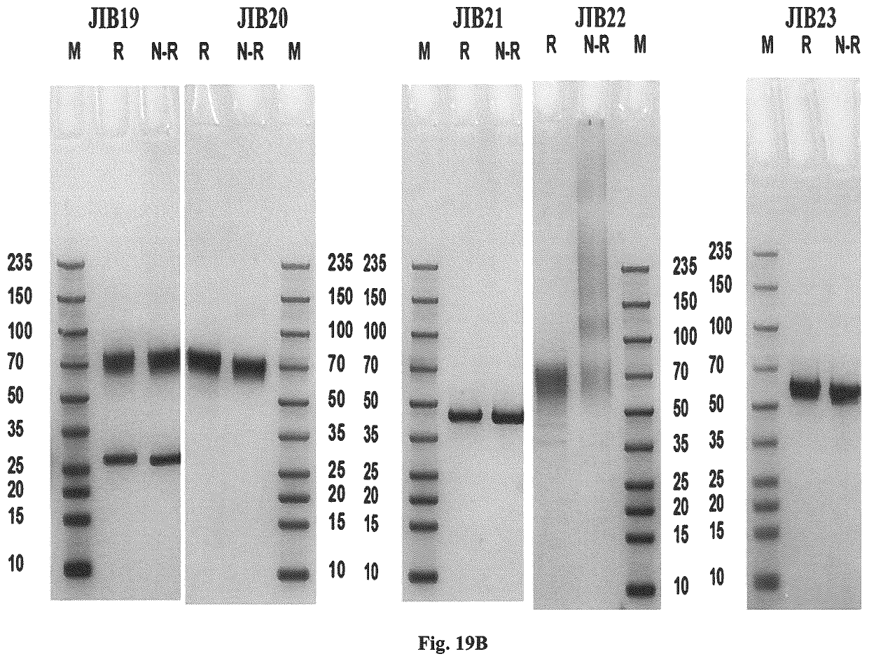

IgRs at 0.1, 1 and 10 nM were evaluated for their ability to bridge HER2+ SKBR3 breast cancer cells with T cells and mediate T cell activation with the presence of 1 nM Trastuzumab and avoid T cell activation in the presence of anti-CD20 Rituximab. As shown in FIG. 17, FIG. 18, FIG. 19A, and FIG. 19B, all 6 designs were capable of driving more T cell activation when combined with Trastuzumab versus an SKBR3 cancer cell line irrelevant IgG, Rituximab, given SKBR3 cells lack CD20. FIG. 16 shows that the anti-TCR V700 fusion exhibited lower potency than the anti-CD3 TR66 or anti-CD3 HzUCHT1 fusions and the TR66 fusion exhibited a higher potency than HzUCHT1 indicative of the importance to optimize around the T cell target, the epitope within a T cell target and the affinity against that T cell target. FIG. 17 shows that the IgR designs with half-life extension strategies such as fusion to an anti-HSA VHH or fusion to an IgG Fc with FcR KO mutations show similar potencies indicating that the addition of a VHH at the N-terminal end of the FcγRIIIA region did not interfere with Trastuzumab IgG-Fc binding and the fusion of an Fc to the C-terminus of the FcγRIIIA region did not interfere with Trastuzumab IgG-Fc binding. FIG. 18 shows that using an anti-Fc VHH instead of an Fc-receptor such as FcγRIIIA can mediate a higher potency indicating the epitope, affinity and stoichiometry of the Fc-binding (FcB) region towards the IgG-Fc are important parameters to optimize (for example FcγR:IgG stoichiometry is 1:1 while anti-Fc VHH:IgG stoichiometry could be 2:1 or 1:1 depending on the epitope). The ability of IgRs in combination with Rituximab in Example 8 and Trastuzumab in Example 9 being able to activate T cells across both blood and solid tumor cancer cells and across 2 different antigen target indicates IgRs can function broadly and independent of the immunoglobulin target antigen and target cell type.

### Example 10. Expression, Purification and Evaluation of FcγR Variants Fused to αCD3

Fc-binding heterologous polypeptides can be expressed and purified as described in Example 1.

The extracellular domain of the human CD16A Fc-receptor (FcγR3A-V158, SEQ ID NO: 1) can be shortened by removal of 15 amino acids on the C-terminus (FcγR3AV-short, SEQ 3), this can then be fused via a (G4S)₃ linker to: a humanized version of an αCD3 ScFv clone HzUCHT1 in the VL-VH orientation using a (G4S)₃ linker to form a FcγR3AV-short/ScFv-HzUCHT1 fusion (SEQ ID NO: 75, SEQ ID NO: 76) as depicted in FIG. 2A. The extracellular domain of the human CD16A Fc-receptor (FcγR3A-V158, SEQ ID NO: 1) with an S197P mutation (FcγR3AV-S197P) and replacing LVGSKNV in domain 2 of FcγR3A with MGKHRY from FcγR1 (SEQ 77), can be fused via a (G4S)₃ linker to: a humanized version of an αCD3 ScFv clone HzUCHT1 in the VL-VH orientation using a (G4S)₃ linker to form a FcγR3A-MGKHRY/ScFv-HzUCHT1 fusion (SEQ ID NO: 78, SEQ ID 79) as depicted in FIG. 2A. The extracellular domain of the human CD64 Fc-receptor (FcγR1, SEQ ID NO: 12) can be truncated to remove domain 3 (FcγR1-D1-D2, SEQ 80), fused via a (G4S)₃ linker to: a humanized version of an αCD3 ScFv clone HzUCHT1 in the VL-VH orientation using a (G4S)₃ linker to form a FcγR1-D1-D2/ScFv-HzUCHT1 fusion (SEQ ID NO: 81, SEQ ID NO: 82) as depicted in FIG. 2A. The extracellular domain of the human CD32A Fc-receptor (FcγR2A-H131, SEQ ID NO: 9) can be fused via a (G4S)₃ linker to: a humanized version of an αCD3 ScFv clone HzUCHT1 in the VL-VH orientation using a (G4S)₃ linker to form a FcγR2AH/ScFv-HzUCHT1 fusion (SEQ ID NO: 83, SEQ ID 84) as depicted in FIG. 2A. The extracellular domain of the human CD32B/C Fc-receptor (FcγR2BC, SEQ ID NO: 11) can be fused via a (G4S)₃ linker to: a humanized version of an αCD3 ScFv clone HzUCHT1 in the VL-VH orientation using a (G4S)₃ linker to form a FcγR2BC/ScFv-HzUCHT1 fusion (SEQ ID NO: 85, SEQ ID NO: 86) as depicted in FIG. 2A. The extracellular domain of the mouse Fc-receptor RIV (FcγRIV, SEQ ID NO: 8) can be fused via a (G4S)₃ linker to: a humanized version of an αCD3 ScFv clone HzUCHT1 in the VL-VH orientation using a (G4S)₃ linker to form a FcγR2BC/ScFv-HzUCHT1 fusion (SEQ ID NO: 87, SEQ ID NO: 88) as depicted in FIG. 2A.

Optionally, other anti-CD3, anti-TCR or other immune cell surface polypeptide binding domains could be used and fused to the FcγR variants described herein. Optionally, the FcγR described herein could be fused in a Fab format with or without lipid conjugation or an Fc fusion format. The function of the IgRs with or without tumor targeting IgGs could be evaluated using a reported assay as described in Examples 8 and 9 or using cytotoxicity assays with primary immune cells.

### Example 11. Expression and Purification of FcγR/αCD3 Multimeric Proteins in Various Fab Formats

Fc-binding multimeric proteins can be expressed and purified as described in Example 1 or optionally a Protein A (for VH3 family heavy chain), Protein L (for VL1, VL3, VL4 family light chain) or anti-CH1 (for any family) affinity chromatography could be employed.

The constant heavy chain CH1 region of a human IgG and optionally some of the hinge (CH1 SEQ ID 89) and the constant kappa light chain (kappa SEQ ID NO: 90) and optionally lambda (LC1 SEQ ID NO: 91, LC2 SEQ ID NO: 92, LC3 SEQ ID NO: 93, LC7 SEQ ID NO: 46), when appropriate, can be fused with VH and VL, respectively, of immune cell surface targeting antigen binding domains. Optionally, the interchain disulfide forming Cysteines on the constant heavy chain (C233S, CH1-NoDS SEQ ID NO: 94) and constant light chain (C214S, kappaNoDS SEQ ID NO: 95, hLC1NoDS SEQ ID NO: 96, hLC2NoDS SEQ ID NO: 97, hLC3NoDS SEQ ID NO: 98, hLC7NoDS SEQ ID NO: 99) to Serine or the like. Optionally, in addition to mutating the interchain disulfide forming Cysteines to Serine or the like, additional buried disulfide forming cysteines could be introduced in both the constant heavy chain (F174C and C233S, CH1-bDS SEQ ID NO: 100) and constant light chains (S176C and C214S, kappabDS SEQ ID NO: 101, hLC1bDS SEQ ID NO: 102, hLC2bDS SEQ ID NO: 103, hLC3bDS SEQ ID NO: 104, hLC7bDS SEQ ID NO: 105).

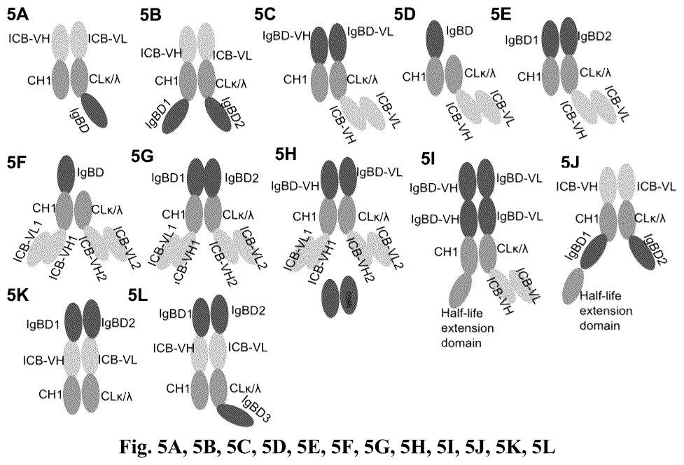

The VH/VL pair of HzUCHT1 can be fused to the CH1 heavy chain and kappa light chain with the extracellular domain of Fc receptor such as FcγR3AV fused to the C-terminus of the constant light chain as depicted in FIG. 5A or optionally or additionally the constant heavy chain via a (G4S)₃ linker as depicted in FIG. 5B (CH1NoDS-HzUCHT1, SEQ ID NO: 106, CH1NoDS-HzUCHT1/FcγR3AV-short, SEQ ID NO: 107, kappaNoDS-HzUCHT1/FcγR3AV-short, SEQ ID NO: 108) or optionally the N-terminus of the variable heavy chain or variable light chain via a (G4S)₃ linker. FcγR3AV can be fused to the N-terminus of the CH1 domain and optionally with HzUCHT1 ScFv fused to the C-terminus and co-expressed with constant light chain with HzUCHT1 ScFv fused to the C-terminus and optionally with FcγR3AV fused to the N-terminus as depicted in FIGS. 5D, 5E, 5F and 5G (FcγR3AV-short/CH1bDS SEQ ID NO: 109, FcγR3AV-short/CH1bDS/ScFv-HzUCHT1 SEQ ID NO: 110, kappabDS/ScFv-HzUCHT1 SEQ ID NO: 111, FcγR3AV-short/kappabDS/ScFv-HzUCHT1: 112). The function of the IgRs with or without tumor targeting IgGs could be evaluated using a reported assay as described in Examples 8 and 9 or using cytotoxicity assays with primary immune cells.

### Example 12. Expression, Purification and Lipid Conjugation of FcγR/αCD3 Multimeric Polypeptides in Various Fab Formats

Fc-binding multimeric proteins can be expressed and purified as described in Example 11.

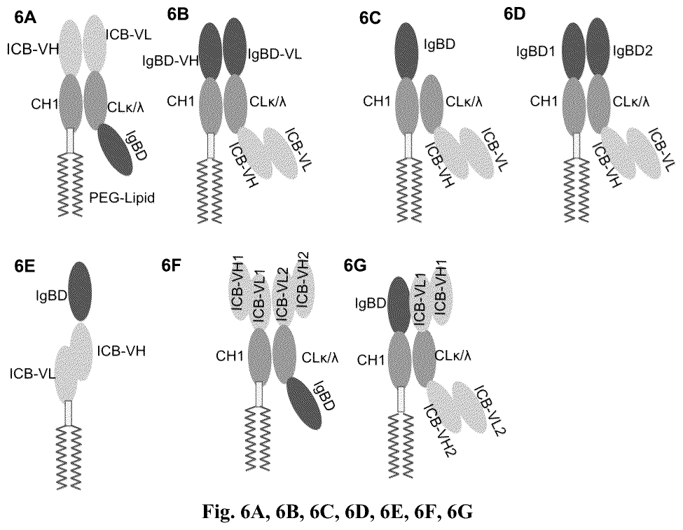

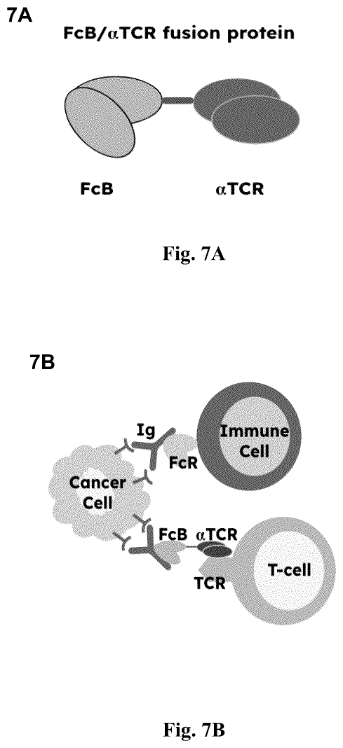

The constant heavy CH1 domain with the native hinge region up the first cysteine and with the interchain disulfide mutated to Serine (C233S, CH1-NoDS-Cys SEQ ID NO: 113) or the like can be paired with the NoDS light chain sequences and VH/VL pairs against immune cell surface proteins as described in Example 11. Optionally, the constant heavy CH1 domain with the native hinge region up the first cysteine and with the interchain disulfide mutated to Serine can have an additional mutation introduced in both the heavy chain (F174C and C233S, CH1-bDS-Cys SEQ ID NO: 114) and light chain sequences to generate Fabs with buried disulfides as described in Example 11. PEG-lipid can be covalently attached to the C-terminal cysteine on the constant heavy chain as depicted in FIG. 6A, FIG. 6B, FIG. 6C, FIG. 6D, FIG. 6F, FIG. 6G or the C-terminal cysteine of an ScFv as depicted in FIG. 6E.

The VH/VL pair of HzUCHT1 can be attached to their corresponding CH1 heavy chain and kappa light chain and fused to an immunoglobulin binding domain such as FcγR3AV at the C-terminus of the constant heavy chain via a (G4S)₃ linker (HzUCHT1-CH1NoDS-Cys, SEQ ID NO: 115, HzUCHT1-CH1bDS-Cys, SEQ ID NO: 116, FcγR3AV-short/kappaNoDS-HzUCHT1, SEQ ID 108, FcγR3AV-short/kappabDS-HzUCHT1, SEQ ID NO: 117) as depicted in FIG. 6A. The function of the IgRs with or without tumor targeting IgGs could be evaluated using a reported assay as described in Examples 8 and 9 or using cytotoxicity assays with primary immune cells.

After expression and purification of a Fab fusion with a C-terminal cysteine, lipid conjugation can be performed. First, make a stock of Maleimide-PEG2K-DSPE/MeOH-PEG2K-DSPE, dissolve Maleimide-PEG2K-DSPE to 3.4 mM (10 mg/L) in 1 mM Citrate pH 6.7, dissolve MeOH-PEG2K-DSPE to 3.4 mM (9.54 mg/mL) in 1 mM Citrate pH 6.7. These stocks can be frozen and stored at −80C individually or after mixing at desired molar ratio. For a 2:3 Mal:MeOH, given the stocks are both at 3.4 mM one can perform volumetric ratio mixing, for instance 200 ul of Mal stock+300 ul MeOH stock would get you 500 ul at a 2:3 molar ratio. If the Fab is not around 3 mg/mL already, concentrate the protein with a 10 kD ultracel regenerated cellulose membrane or the like, use fresh PBS for recovery, spike with 0.5 M EDTA pH 7.5 for final of PBS+5 mM EDTA pH 7.5.

For the reduction, dissolve TCEP to 100 mM in PBS+5 mM EDTA, spike TCEP concentrate for final concentration of around 0.2 mM in protein solution, reduce at room temp for around 1.5 hr. After reduction, use a 40K Zeba desalting column to remove free cysteine or other small molecules and exchanging the Fab into PBS+5 mM EDTA. After Zeba, take A280 by nanodrop or the like and calculate protein concentration so one can calculate how much Mal-PEG-DSPE/MeOH-PEG-DSPE to add for around 2:1 ratio of MAL:Protein, this is equivalent to PEG-DSPE to protein ratio of around 5:1.

For the reaction, add protein to MAL/MeOH mixture, pipette mix, incubate for around 3 hr at 37° C. After reaction, spike 1:10 v/v with 15 mM Cysteine (for final of 1.5 mM cysteine post dilution), incubate around 15 min 37° C. to quench unreacted Mal-PEG-DSPE.

For purification, dilute with 25 mM HEPES 150 mM NaCl pH 7.5 (HBS), concentrate with 100 kD UF membrane, dilute >25×, repeat for serial concentration/dilution 3 times, this lets unreacted protein pass through the filter while lipid conjugated protein micelles get retained. Carefully recover protein-micelles from membrane, with 1-2 washes using 10-15 ul, ideally final volume is only 35-50 ul, measure volume by pipette, measure A280 by nanodrop or cuvette material can be recovered from. Run SDS-PAGE with original material non-reduced and reduced and final conjugate reduced, one should see a shift in the heavy chain band between the conjugated Fab and PEG-lipid conjugated Fab. Optionally, a similar PEG-lipid conjugation and purification procedure can be performed when a Fc-binding-domain/ScFv fusion protein contains a C-terminal cysteine.

### Example 13. Expression and Purification of Multiple-FcRs or Fc-Binding Domains Fused to αCD3

Fc-binding heterologous polypeptides and heteromultimeric proteins can be expressed and purified as described in Example 1 and Example 11.

Multiple immunoglobulin-binding domains of the same type or different types can be fused with an ScFv or Fab against an immune cell surface protein (as depicted in 4D, 4E, 4F, 4G) optionally with or without an half-life extension domain such as anti-HSA (as depicted in FIG. 3B) or in an Fc-Fc heterodimeric protein format. FcγR3A can be fused to a HzUCHT1 ScFv followed by another FcγR3A (FcγR3AV-short/ScFv-HzUCHT1/FcγR3AV-short, SEQ ID NO: 118) as depicted in FIG. 2C with an optional domain order. FcγR2A can be fused to a HzUCHT1 ScFv followed by FcγR3A (FcγR2AH131/ScFv-HzUCHT1/FcγR3AV-short, SEQ ID NO: 119) as depicted in FIG. 2C with an optional domain order. FcαRI can be fused to a FcγR3A followed by HzUCHT1 ScFv (FcαRI/FcγR3AV-short/ScFv-HzUCHT1, SEQ ID NO: 120) as depicted in FIG. 2C. Two tandem domain 1 of FcαRI can be fused to HzUCHT1 ScFv (FcαRI-D1/FcαRI-D1/ScFv-HzUCHT1, SEQ ID NO: 121) as depicted by FIG. 2C and optionally with FcγR3A. Anti-Fc VHH can be fused to another anti-Fc followed by HzUCHT1 ScFv (anti-Fc/anti-Fc/ScFv-HzUCHT1, SEQ ID NO: 122) as depicted in FIG. 2C. Anti-Fc VHH can be fused to a FcγR3A followed by HzUCHT1 ScFv (anti-Fc/FcγR3AV-short/ScFv-HzUCHT1, SEQ ID NO: 123) as depicted in FIG. 2C. FcγR3A can be fused to the C-terminus of CH1/HzUCHT1 heavy chain (CH1-HzUCHT1/FcγR3AV-short, SEQ ID NO: 124) and the C-terminus of the kappa/HzUCHT1 light chain (kappa-HzUCHT1/FcγR3AV-short, SEQ ID NO: 125) as depicted in FIG. 5B. FcγR3A can be fused to the N-terminus of IgG1-LALAPA-knob Fc forming FcγR3AV-short/Fc-LALAPA-knob, SEQ ID NO: 126 and optionally the N-terminus of a ScFv-HzUCHT1-Fc-LALAPA-hole SEQ ID 128 forming FcγR3AV-short/kappa-HzUCHT1-Fc-LALAPA-hole, SEQ ID NO: 129 to produce a hetero-multimeric proteins with one or two immunoglobulin binding domains as depicted in FIG. 4C and FIG. 4D. The function of the IgRs with or without tumor targeting IgGs and/or IgAs when appropriate could be evaluated using a reported assay as described in Examples 8 and 9 or using cytotoxicity assays with primary immune cells.

FcγR3A can be fused to the C-terminus via a (G4S)₃ linker to a partial human IgG1 heavy chain with mutations: C233S to prevent disulfide formation; T366S, L368A, Y407V to produce the “hole” Fc heavy chain; and L234A, L235A and P329A to ablate Fc receptor binding; forming an FcγR3A-Fc polypeptide (FcγR3A-short/hIgG1-Fc-LALAPA-hole SEQ ID NO: 41). The other heavy chain can comprise of a variable heavy chain for the αCD3 clone HzUCHT1 and a human IgG1 constant heavy chain with mutations: T366W to produce the “knob” Fc heavy chain; and L234A, L235A and P329A to ablate Fc receptor binding; forming an HzUCHT1 human IgG1 heavy chain (HzUCHT1-HC-Fc-LALAPA-knob, SEQ ID NO: 127. The light chain comprised of a variable light chain for the αCD3 clone HzUCHT1 and a human constant kappa light chain with an FcγR3AV fused to the C-terminus via a (G4S)3 linker forming an HzUCHT1 human light chain FcγR3AV fusion (kappa-HzUCHT1/FcγR3AV-short, SEQ ID NO: 125) as depicted in FIG. 4G. Expression of these 3 chains would produce a hetero-multimeric protein with two immunoglobulin-binding domains.

Using a Fab format, FcγR3A can be fused to the C-terminus a of a CH1 (CH1-HzUCHT1/FcγR3AV-short, SEQ ID NO: 124) and the C-terminus of a constant light chain (kappa-HzUCHT1/FcγR3AV-short, SEQ ID NO: 125) to form a hetero-multimeric protein with two immunoglobulin domains as depicted in FIG. 5B.

### Example 14. Expression and Purification of Immunoglobulin Binding Domains Fused to αCD3

Immunoglobulin-binding heterologous polypeptides and heteromultimeric proteins can be expressed and purified as described in Example 1 and Example 11.

Immunoglobulin binding domains such as anti-CH1 VHH (VHH-CH128 SEQ ID 130) can be fused to various immune cell binding domains with specificity towards CD3, TCR or other immune cell surface polypeptides using a (G4S)₃ linker to form VHH-CH128/ScFv fusions. Fusing the anti-CD3 HzUCHT1 ScFv to the CH128 VHH via a (G4S)₃ linker forms CH128/ScFv-HzUCHT1 SEQ ID NO: 131. An avimer against the Fc region of an IgG (AVIG SEQ ID 132) can be fused to HzUCHT1 ScFv to form AVIG/ScFv-HzUCHT1 SEQ ID NO: 133. An anti-IgG VH/VL pair (17F12 VH SEQ ID NO: 134, 17F12 VL SEQ ID NO: 135) can be fused to an immune cell targeting domain in the form of an ScFv (ScFV-17F12 SEQ ID 136) forming ScFv-17F12/ScFv-HzUCHT1: 137 or optionally as a Fab with one immune cell targeting domain attached to the C-terminus of the light constant light chain, heavy chain or both (CH1-NoDS-17F12 SEQ ID NO: 138, CH1-NoDS-17F12/ScFv-HzUCHT1 SEQ ID NO: 140, kappaNoDS-17F12/ScFv-HzUCHT1 SEQ ID NO: 141) as depicted in FIG. 5C and FIG. 5H or an Fc fusion with one or multiple point mutations in the constant heavy chain to significantly reduce 17F12 binding as depicted in FIG. 4A where the IgBD is a whole Fab comprising the 17F12 VH/VL pair attached to their corresponding constant heavy and light chains. Optionally, an anti-IgG Fab with one immune cell targeting domain attached to the C-terminus of the light constant light chain (CH1-NoDS-17F12-Cys SEQ ID NO: 139, kappaNoDS-17F12/ScFv-HzUCHT1 SEQ ID NO: 141) can be conjugated to an lipid via free cysteine at or near the C-terminus as depicted in FIG. 6B. The function of the IgRs with or without tumor targeting IgGs could be evaluated using a reported assay as described in Examples 8 and 9 or using cytotoxicity assays with primary immune cells.

### Example 15. Expression and Purification of FcγR Fused to αCD3 and αTCR

Fc-binding heterologous polypeptides can be expressed and purified as described in Example 1.

An immunoglobulin-binding domain such as the extracellular domain of the human CD16A Fc-receptor with an S197P mutation (FcγR3AV-S197P SEQ ID 23) or optionally with 15 amino acids removed from the C-terminus, can be fused to various VH/VL pairs with specificity towards CD3 and TCR using a (G4S)₃ linker to form FcγR3AV-ScFv fusions: anti-CD3 Hu291 (Hu291-VH SEQ: 142, Hu291-VL SEQ ID NO: 143, ScFv-Hu291 SEQ ID NO: 144, FcγR3AV-short/ScFv-Hu291 SEQ ID NO: 145), anti-CD3 huCLB-T3/4.A (huCLB-VH SEQ ID NO: 146, huCLB-VL SEQ ID NO: 147, ScFv-huCLB SEQ ID NO: 148, FcγR3AV-short/ScFv-huCLB SEQ ID NO: 149), anti-CD3 HuYTH 12.5 (12.5-VH SEQ ID NO: 150, 12.5-VL SEQ ID NO: 151, ScFv-12.5 SEQ ID NO: 152, FcγR3AV-short/ScFv-12.5 SEQ ID NO: 153), anti-CD3 TR66-high affinity (TR66HF-VH SEQ ID NO: 154, TR66HF-VL SEQ ID NO: 155, ScFv-TR66HF SEQ ID NO: 156, FcγR3AV-short/ScFv-TR66HF SEQ ID NO: 157), anti-CD3 G19-4 (G19-4-VH SEQ ID NO: 158, G19-4-VL SEQ ID NO: 159, ScFv-G19-4 SEQ ID NO: 160, FcγR3AVshort/ScFv-G19-4 SEQ ID NO: 161), anti-CD3 BC3 (BC3-VH SEQ ID NO: 162, BC3-VL SEQ ID NO: 163, ScFv-BC3 SEQ ID NO: 164, FcγR3AV-short/ScFv-BC3 SEQ ID NO: 165), anti-CD3 Hz06 (Hz06-VH SEQ ID NO: 166, Hz06-VL SEQ ID NO: 167, ScFv-Hz06 SEQ ID NO: 168, FcγR3AV-short/ScFv-Hz06 SEQ ID NO: 169), anti-CD3 Ly17.2G3 (Ly17-VH SEQ ID NO: 170, Ly17-VL SEQ ID NO: 171, ScFv-Ly17 SEQ ID NO: 172, FcγR3AV-short/ScFv-Ly17 SEQ ID NO: 173), anti-TCR TOL-101 (TOL-VH SEQ ID NO: 174, TOL-VL SEQ ID NO: 175, ScFv-TOL SEQ ID NO: 176, FcγR3AV-short/ScFv-TOL SEQ ID NO: 177), anti-TCR hJOV1 (hJOV1-VH SEQ ID NO: 178, hJOV1-VL SEQ ID NO: 179, ScFv-hJOV1 SEQ ID NO: 180, FcγR3AVshort/ScFv-hJOV1 SEQ ID NO: 181), anti-TCR BMA-031 (BMA-VH SEQ ID NO: 182, BMA-VL SEQ ID NO: 183, FcγR3AV-short/ScFv-BMA SEQ ID NO: 184). Optionally, the VH/VL pairs in this example or other examples could be incorporated in an Fc fusion format as described in Example 3 or a Fab format with or without a C-terminal and with or without lipid conjugation as described in Examples 11 and 12. The function of the IgRs with or without tumor targeting IgGs could be evaluated using a reported assay as described in Examples 8 and 9 or using cytotoxicity assays.

### Example 15. Expression and Purification of FcγR Fused to Co-Stimulation, Immune Checkpoint and Immune Suppression Targets on T Cells

Fc-binding heterologous polypeptides and heteromultimeric proteins can be expressed and purified as described in Example 1 and Example 11.

Immunoglobulin-binding domain Fc fusions can be expressed with immune cell binding domain Fc proteins to generate multiple hetero-multimeric proteins with 1 to 2 immunoglobulin-binding domains combined with 1 to 2 immune cell binding domains as depicted in FIG. 4A, FIG. 4B, FIG. 4C, FIG. 4D, FIG. 4E, FIG. 4F, FIG. 4G, FIG. 4H, FIG. 4I, and FIG. 4J. 3 polypeptide chains can assemble into one soluble protein comprising two heterodimer forming heavy chains be assembled using knob-in-hole mutations and a light chain that assembles with one of the two heterodimeric heavy chains. One heavy chain can comprise of an immunoglobulin binding domain such as human CD16A Fc-receptor fused via a (G4S)₃ linker to a partial human IgG1 heavy chain with mutations: C233S to prevent disulfide formation; T366S, L368A, Y407V to produce the “hole” Fc heavy chain; and L234A, L235A and P329A to ablate Fc receptor binding; forming an FcγR3A-Fc polypeptide. The other heavy chain can comprise of a variable heavy chain of various T cell co-stim and immune checkpoint targeting clones (anti-CD137 5B9, Urelumab, P566 (Utomilumab), Hz4B4-2; anti-CD28 2E12, TGN1412, 9.3; anti-PD1 Nivolumab, Pembrolizumab; anti-CTLA4 Ipilizumab; anti-CD11 a hMHRM24; anti-CD18 rhuMab) and a human IgG1 constant heavy chain with mutations: T366W to produce the “knob” Fc heavy chain; and L234A, L235A and P329A to ablate Fc receptor binding. The light chain can comprise of the corresponding variable light chains of various T cell co-stim and immune checkpoint targeting clones and a human constant kappa or lambda light chain as appropriate. Optionally, these T cell co-stimulation or immune checkpoint targeting clones can be produced as ScFvs and fused to the C-terminus or N-terminus of an immunoglobulin binding domain, the light chain of an another T cell targeting Fab or Fc-fusion or an anti-T cell targeting ScFv such as anti-CD3 or TCR can be fused to the C-terminal light chain of an anti-T cell immune checkpoint or costimulation light chain for example CH1NoDS-17F12/ScFv-9.3 SEQ ID NO: 220, kappaNoDS-17F12/ScFv-HzUCHT1 SEQ ID NO: 141, FIG. 5H; FcγR3AV-short/CH1bDS/ScFv-9.3 SEQ ID NO: 221, kappabDS/ScFv-HzUCHT1 SEQ ID NO: 111, FcγR3AV-short/kappabDS/ScFv-HzUCHT1 SEQ ID 112 FIG. 5F, FIG. 5G). The function of the IgRs with or without tumor targeting IgGs could be evaluated using a reported assay as described in Examples 8 and 9 or using cytotoxicity or activation assays with primary immune cells.

### Example 16. Expression and Purification of FcγR Fused to Effector Function, Co-Stimulation, Immune Checkpoint Targets on Myeloid, B Cells and NK Cells

Fc-binding heterologous polypeptides and heteromultimeric proteins can be expressed and purified as described in Example 1 and Example 11.

Immunoglobulin binding domains such as Fc receptors, anti-Fc, anti-CH1 and anti-IgG can be fused to immune cell surface polypeptide binding domains on Myeloid cells, B cells and NK cells in an ScFv fusion format or optionally can be could be incorporated in an Fc fusion format as described in Example 3 (with mutations to knock out anti-CH1 or anti-IgG binding as appropriate) or a Fab format (with mutations to knock out anti-CH1 or anti-IgG binding as appropriate) with or without a C-terminal and with or without lipid conjugation as described in Examples 11 and 12. The function of the IgRs with or without tumor targeting IgGs can be evaluated using reporter assays similar to those in Example 8 and 9, primary cell activation assays, cytotoxicity assays or phagocytosis assays. FcγR3a can be fused to anti-CD89 VH/VL pairs (14A8, A77, 8D2) to target Myeloid cells. FcγR3a can be fused to anti-CD40 VH/VL pair (40.2.220), CD40L, anti-CD79 (SN8, 2F2) to target B cells. FcγR3a can be fused to anti-NKp46 VH/VL pair (NKp46-1, NKp46-4) to target NK cells.

### Example 17. Expression and Purification of Half-Life Extension Domains Fused to Immunoglobulin-Binding and Immune Cell Binding Domains

Fc-binding heterologous polypeptides and heteromultimeric proteins can be expressed and purified as described in Example 1 and Example 11.

Anti-HSA or HSA can be fused to the N or C terminus or optionally between immunoglobulin binding domains and immune cell binding domains as depicted in FIG. 3A, FIG. 3B, and FIG. 3C. Anti-HSA VHH or HSA (SEQ ID NO: 266) can be fused to the C-terminus of FcγR3A fused to anti-CD3 ScFv UCHT1 fused to another FcγR3A to form anti-HSA/FcγR3A/ScFv-HzUCHT1/FcγR3A SEQ ID NO: 267 and HSA/FcγR3A-short/ScFv-HzUCHT1/FcγR3A SEQ ID NO: 268. HSA can also be fused to FcγR3A-short/ScFv-HzUCHT1 to form HSA/FcγR3A-short/ScFv-HzUCHT1 SEQ ID 269. Optionally, anti-HSA can be fused to one immunoglobulin-binding domain and two immune cell binding domains (anti-HSA/FcγR3A/ScFv-HzUCHT1/ScFv-9.3 SEQ ID NO: 270)

### Example 18. Expression, Purification and Evaluation of FcγR Variants, IgG Binding Variants and Multimeric Proteins in Various Formats Fused to αCD3

IgG-binding heterologous polypeptides and heteromultimeric proteins can be expressed and purified as described in Example 1 and Example 11. IgG-binding multimeric proteins can be expressed and purified as described in Example 1 or optionally a Protein A (for VH3 family heavy chain), Protein L (for VL1, VL3, VL4 family light chain) or anti-CH1 (for any family) affinity chromatography could be employed.

The FcγR3AV-short/ScFv-HzUCHT1-his fusion (JIB15, SEQ ID NO: 76), FcγR3A-MGKHRY/ScFv-HzUCHT1-his fusion (JIB16, SEQ ID 79), FcγR1-D1-D2/ScFv-HzUCHT1 fusion (JIB17, SEQ ID NO: 82), FcγR2AH/ScFv-HzUCHT1-his fusion (JIB18, SEQ ID 84) and FcγR2BC/ScFv-HzUCHT1 fusion (JIB23, SEQ ID NO: 86) from Example 10 were produced as well as the VHH-CH128/ScFv-HzUCHT1-his fusion using SEQ ID NO: 131, and SEQ ID NO: 21 from Example 14 was produced as JIB21 (SEQ ID NO: 278) as depicted in FIG. 2A.

The VH of HzUCHT1 was fused to the CH1 heavy chain and his tag (CH1NoDS-HzUCHT1 SEQ ID NO: 106; his SEQ ID NO: 21) forming CH1NoDS-HzUCHT1-his (SEQ: ID 279) and the VL of HzUCHT1 was fused to the kappa light chain (kappaNoDS, SEQ ID NO: 108) with the extracellular domain of Fc receptor FcγR3AV-S197P (SEQ ID NO: 23) fused to the C-terminus of the constant light chain via a (G4S)₃ (kappaNoDS-HzUCHT1-FcγR3AV-S197P, SEQ ID NO: 280) as depicted in FIG. 5A to form JIB19 (SEQ ID NO: 279 and SEQ ID NO: 280) or optionally or additionally the constant heavy chain via a (G4S)₃ linker making CH1NoDS-HzUCHT1-FcγR3AV-S197P-his, SEQ ID NO: 281 (CH1NoDS-HzUCHT1 SEQ ID NO: 106; FcγR3AV-S197P SEQ ID NO: 23, his SEQ ID NO: 21) as depicted in FIG. 5B to form JIB20 (SEQ ID 280, SEQ ID 281).

The anti-IgG clone 17F12, a known human IgG1, IgG2, IgG3 and IgG4 binder that cross reacts with non-human primate IgG, was fused to human CH1 heavy chain (SEQ ID 138) with a his tag (SEQ ID 21) formed CH1NoDS-17F12-his (SEQ ID 282) and kappa light chain (kappaNoDS-17F12, SEQ ID NO: 112) and a humanized version of an αCD3 ScFv clone HzUCHT1 in the VL-VH orientation using a (G4S)₃ linker was fused to the C-terminus of the constant light chain via (G4S)₃ linker to form kappaNoDS-17F12/ScFv-HzUCHT1 (SEQ ID NO: 141) generating to heterodimeric polypeptide, JIB22 (SEQ ID NO: 282, SEQ ID NO: 141) as depicted in FIG. 5C.

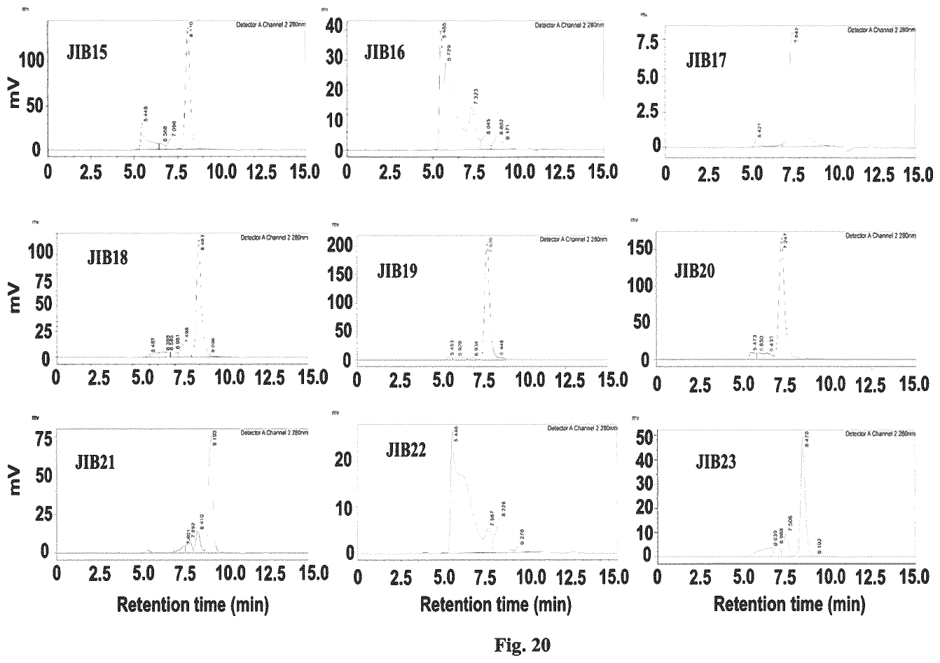

FIG. 19A, FIG. 19B, and FIG. 20 and Table 8 show the proteins were well expressed and were of high to reasonable purity by SDS-PAGE and SEC with exception of JIB16 and JIB22 where their % Monomer by SEC was 4.3% and 13.3% respectively. Interestingly, when comparing their SDS-PAGE profiles, it can be observed that the anti-IgG 17F12 molecule, JIB22, has laddering even when the sample was reduced and denatured which indicates that the binding regions of the VH/VL pair may be binding a portion a constant region.

TABLE 8
Protein Expression and % Monomer by
SEC for IgR produced in Example 18.

| Protein | CHO (mg/L) | SEC % Monomer |
|---|---|---|
| JIB15 | 215 | 69.6% |
| JIB16 | 91 | 4.3% |
| JIB17 | 11 | 96.0% |
| JIB18 | 88 | 74.6% |
| JIB19 | 170 | 89.4% |
| JIB20 | 184 | 84.9% |
| JIB21 | 58 | 67.0% |
| JIB22 | 70 | 13.3% |
| JIB23 | 101 | 63.8% |

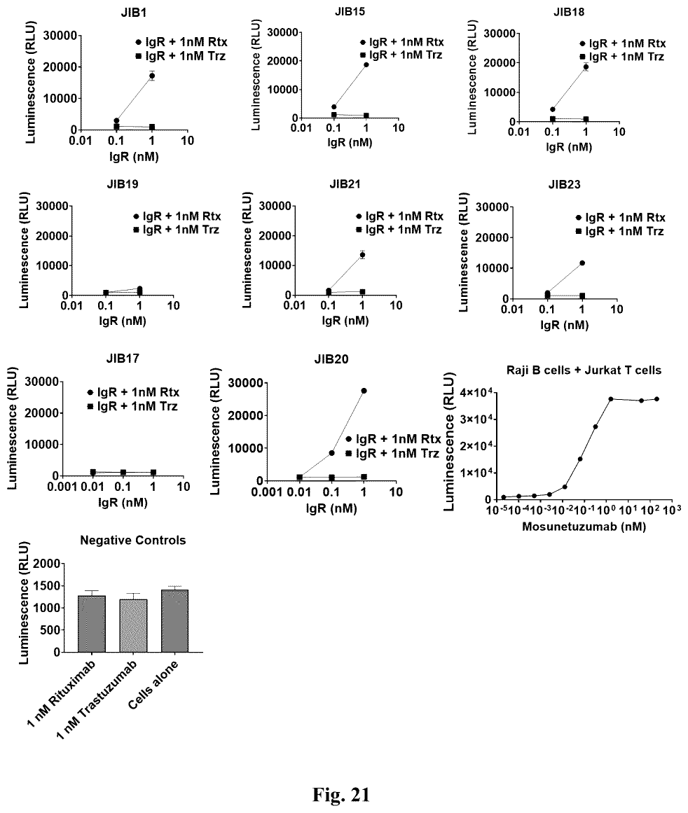

Next, IgRs at 0.1 and 1 nM were evaluated for their ability to bridge CD20+ Raji B cells with T cells and mediate T cell activation in the presence of 1 nM Rituximab, avoid T cell activation in the presence of 1 nM Trastuzumab or with Mosunetuzumab run as a positive control (FIG. 21) using the method described in Example 8. The use of FcγR3A-short in JIB15 did not reduce its potency when compared to the non-truncated FcγR3A form in JIB1 indicating a FcγR3A amino acids near the C-terminus of the extracellular domain, including the lacking the ADAM17 cleavage site are not necessary for IgG binding. The FcγR2A (JIB18), FcγR2BC (JIB23), and anti-CH1 VHH (JIB21) comprising fusions were able to mediate T cell activation in the presence of Rituximab that was substantially greater than in the presence of Trastuzumab indicating IgRs can leverage multiple Fc receptor types that would block cognate immunoglobulin Fc receptor engagement in vivo and IgRs can leverage IgG binders that would not block cognate immunoglobulin Fc receptor engagement in vivo. In contrast, to the FcγR2A-based JIB18 and FcγR2BC-based JIB23, the similarly designed IgR comprising FcγR1 lacking domain 3 (JIB17) did not mediate substantial Rituximab signal over background activation with Trastuzumab indicating the removal of domain 3 in FcγR1 substantially reduced its function. In contrast to the FcγR3A and HzUCHT1 comprising fusions such as JIB1 and JIB15, we observed reduced potency for the HzUCHT1 Fab fusion with one FcγR3A fused to C-terminus of the constant light chain (JIB19). Given this result, we were surprised to observe an IgR identical to JIB19 but with a second FcγR3A fused to the C-terminal constant constant heavy chain, for a total of 2 FcγR3A per IgR molecule (JIB20), exhibited substantial activation at 1 and 0.1 nM Rituximab which was greater than JIB1, JIB15 or JIB19 and it was substantially greater than in the presence of the non-target specific Trastuzumab and without elevated levels of nonspecific activation from Trastuzumab relative to cells alone. These results indicate having 2 or more IgG binding domains can have enhanced potency over an IgR molecule with only one IgG binding domain.

### Example 19. Expression and Purification of Variant FcRs, IgG-Binding or and Fc-Binding Domains Fused to αCD3

IgG and Fc-binding heterologous polypeptides and heteromultimeric proteins can be expressed and purified as described in Example 1 and Example 11.

The wild type VH (SEQ ID NO: 134) and a mutated form of the VL to improve expression and stability (17F12QUAD-VL, SEQ ID NO: 283) of anti-IgG clone 17F12 were fused to a mutated form of the CH1 constant heavy chain comprising P126S and K213E (Kabat numbering) to ablate 17F12 self-binding (C4CH1-bDS, SEQ ID NO: 284) to form (C4CH1-bDS-17F12-his, SEQ ID NO: 285) and kappa light chain (kappabDS-17F12QUAD, SEQ ID NO: 286) and a humanized version of an αCD3 ScFv clone HzUCHT1 in the VL-VH orientation using a (G4S)₃ linker was fused to the C-terminus of the constant light chain to form (kappa-bDS-17F12QUAD-ScFv-HzUCHT1, SEQ ID NO: 287) generating the heterodimeric polypeptide, JIB33 (SEQ ID NO: 285 and 287) as depicted in FIG. 5C.

The wild type anti-Fc clone HP6017 is known to be capable of binding human IgG1, IgG2, IgG3, IgG4 and non-human primate IgG. HP6017-VH, SEQ ID NO: 288 was fused to a buried disulfide CH1 heavy chain (CH1-bDS, SEQ ID NO: 100) to form CH1-bDS-HP6017-his (SEQ ID NO: 289) and the wild type VL (HP6017-VL, SEQ ID NO: 290) was fused to a buried disulfide kappa light chain (kappabDS, SEQ ID NO: 101) to form kappabDS-HP6017 (SEQ ID NO: 291) and a humanized version of an αCD3 ScFv clone HzUCHT1 in the VL-VH orientation using a (G4S)₃ linker was fused to the C-terminus of the constant light chain to form (kappa-bDS-HP6017/ScFv-HzUCHT1, SEQ ID NO: 292) generating to heterodimeric polypeptide, JIB34 (SEQ ID NO: 289 and 292) as depicted in FIG. 5C.

An anti-Fc avimer, AVIG SEQ ID NO: 133 from Example 14 was fused to a his tag forming AVIG/ScFv-HzUCHT1-his fusion, JIB35 (SEQ ID NO: 293) as depicted in FIG. 2A.

The extracellular domain of the human CD32A Fc-receptor comprised point mutations R56H, K118N, T120V, L160Q and V172E, according to the residue number in SEQ ID NO: 9, (FcγR2A_mut, SEQ ID NO: 294) for increased Fc affinity and was fused via a (G4S)₃ linker to a humanized version of an αCD3 ScFv clone HzUCHT1 in the VL-VH orientation using a (G4S)₃ linker to form a FcγR2A_mut/ScFv-HzUCHT1-his fusion, JIB36 (SEQ ID 295) as depicted in FIG. 2A.

The extracellular domain of the human CD16A Fc-receptor comprised point mutations K122N, T124/Q176E, according to the residue number in SEQ ID NO: 1 (FcγR3A_mut1, SEQ ID NO: 296) for increased Fc affinity and was fused via a (G4S)₃ linker to a humanized version of an αCD3 ScFv clone HzUCHT1 in the VL-VH orientation using a (G4S)₃ linker to form a FcγR3A_mut1/ScFv-HzUCHT1-his fusion, JIB37 (SEQ ID 297) as depicted in FIG. 2A.

The extracellular domain of the human CD16A Fc-receptor comprised point mutations 190R, T118K, A119 and Y134F, according to the residue number in SEQ ID NO: 1 FcγR3A_mut2, SEQ ID NO: 298) for varied Fc affinity and was fused via a (G4S)₃ linker to a humanized version of an αCD3 ScFv clone HzUCHT1 in the VL-VH orientation using a (G4S)₃ linker to form a FcγR3A_mut2/ScFv-HzUCHT1-his fusion, JIB38 (SEQ ID 299) as depicted in FIG. 2A.

Domain 2 of the extracellular domain of the human CD64 Fc-receptor was fused with Domain 1 of the CD16A Fc-receptor to form FcγR1-D1-FcγR3A-D2, SEQ ID NO: 300) and was fused via a (G4S)₃ linker to a humanized version of an αCD3 ScFv clone HzUCHT1 in the VL-VH orientation using a (G4S)₃ linker to form a FcγR1-D1-FcγR3A-D2/ScFv-HzUCHT1-his fusion, JIB39 (SEQ ID 301) as depicted in FIG. 2A.

The VH/VL pair of HzUCHT1 was fused to the CH1 heavy chain and kappa light chain with the extracellular domain of Fc receptor FcγR3AV fused to the N-terminus of the variable heavy chain (FcγR3AV-S197P/HzUCHT1-CH1-bDS-his, SEQ ID NO: 302) and variable light chain FcγR3AV-S197P-HzUCHT-kappa-bDS SEQ ID NO: 303) via a (G4S)₃ linker and the chains SEQ ID 302 and 303 were co-expressed to produce JIB40 as shown in FIG. 5K.

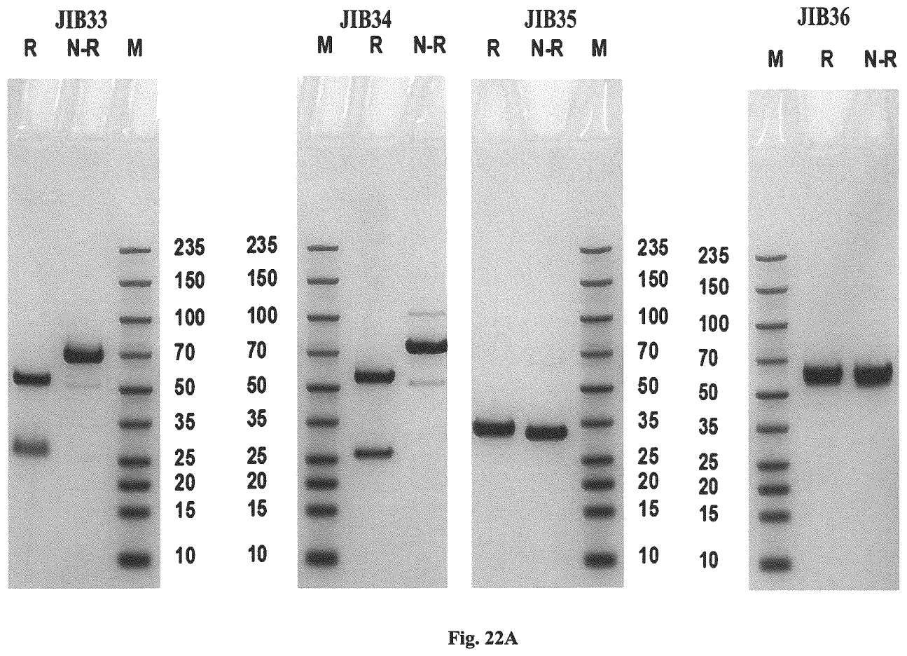

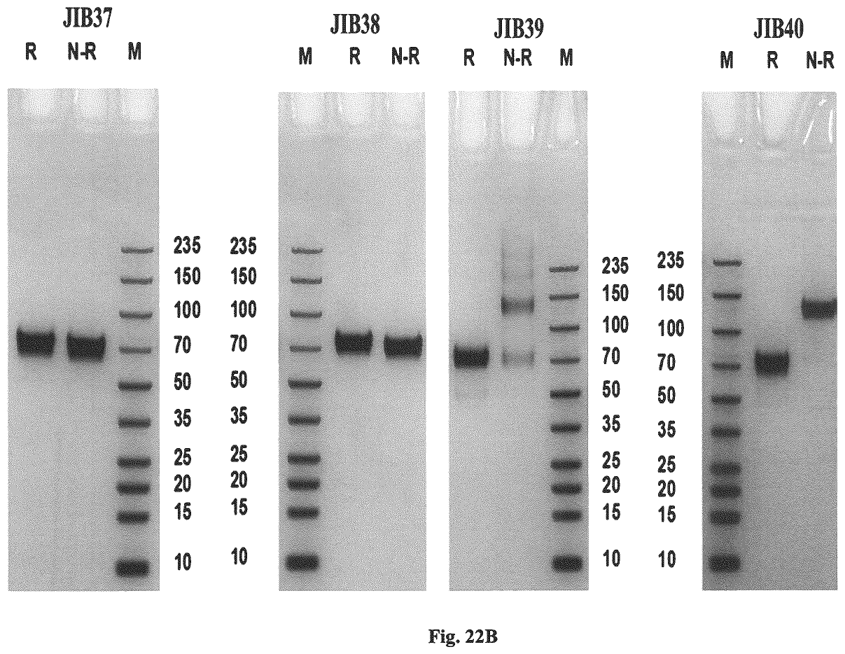

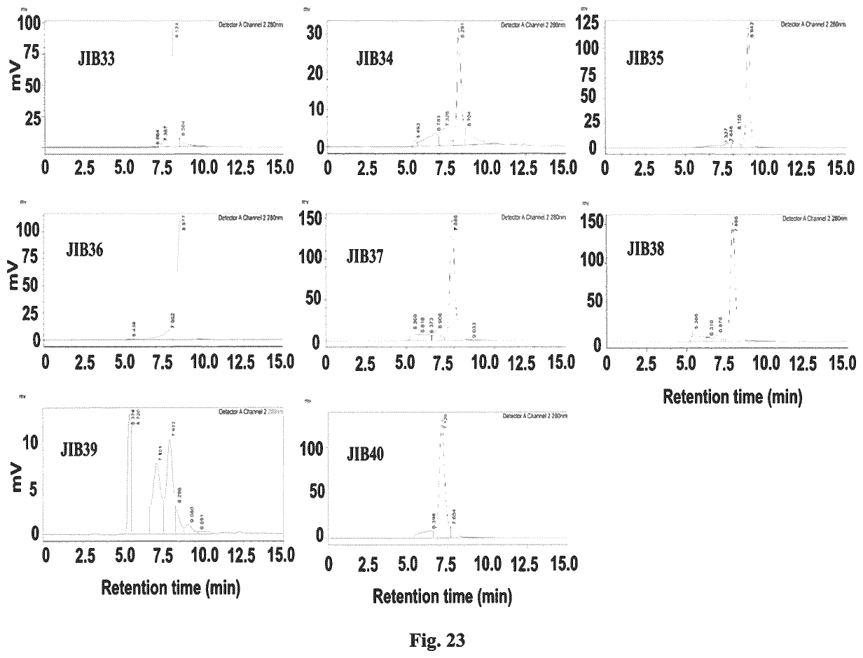

FIG. 22A, FIG. 22B, FIG. 23 and Table 9 show the proteins were well expressed and were of high purity by SDS-PAGE and SEC with exception of JIB34 and JIB39 as shown in Table 2 below where their % Monomer by SEC was 51.8% and 21.1% respectively. Interestingly, the 17F12 variant, JIB33, comprising mutations in its CH1 constant domain to prevent self-binding and in its VL to improve expression did not exhibit laddering by SDS-PAGE in the reduced lane (FIG. 22A and FIG. 22B) in contrast to its wild type counterpart, JIB22. Additionally, JIB33 had substantially improved % Monomer by SEC relative to JIB22 with 86.8% vs. 13.3%, respectively. These results indicate the point mutations P126S and K213E in the constant CH1 domain ablated JIB33 self-binding and the 17F12 clone's binding epitope is in the CH1 domain. Despite a modest purity by SEC, the SDS-PAGE of the HP6017 protein JIB34 did not have laddering in the reduced lane (FIG. 22A and FIG. 22B) unlike that of the wild type 17F12 JIB22 indicating it does not self-bind when it only comprises CH1 and kappa constant regions and lacks an Fc region, consistent with it being a known human IgG1, IgG2, IgG3 and IgG4 Fc binder. The JIB39, FcγR1-D1-FcγR3A-D2/ScFv-HzUCHT1-his fusion had an SDS-PAGE profile indicative of disulfide scrambling by laddering in the non-reduced lane.

TABLE 9
Protein Expression and % Monomer by
SEC for IgR produced in Example 19.

| Protein | CHO (mg/L) | SEC % Monomer |
|---|---|---|
| JIB33 | 87 | 86.8% |
| JIB34 | 74 | 51.8% |
| JIB35 | 68 | 78.4% |
| JIB36 | 140 | 85.5% |
| JIB37 | 140 | 77.8% |
| JIB38 | 103 | 80.8% |
| JIB39 | 52 | 21.1% |
| JIB40 | 99 | 81.9% |

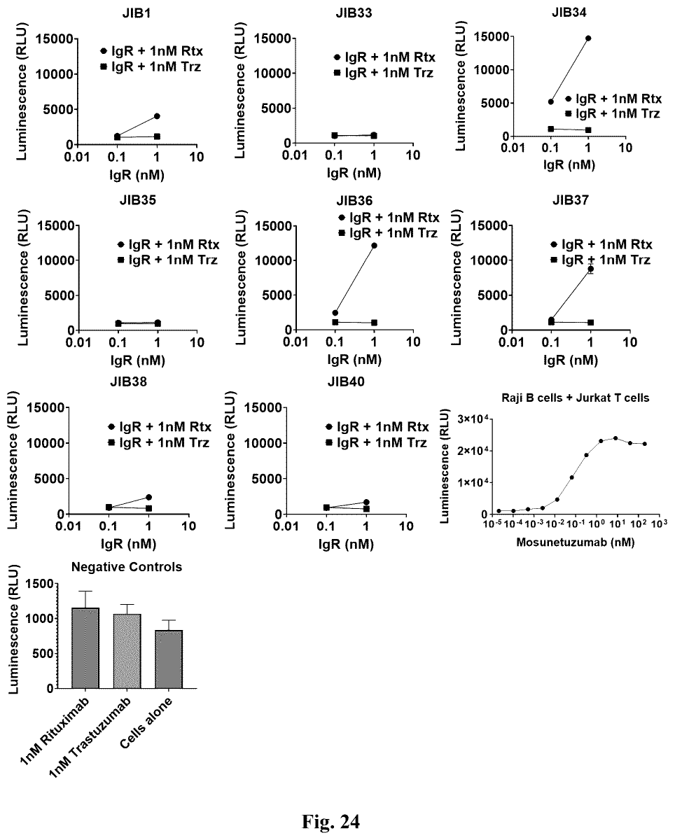

Next, IgRs at 0.1 and 1 nM were evaluated for their ability to bridge CD20+ Raji B cells with T cells and mediate T cell activation in the presence of 1 nM Rituximab, avoid T cell activation in the presence of 1 nM Trastuzumab or with Mosunetuzumab run as a positive control (FIG. 24) using the method described in Example 8. The 17F12 variant HzUCHT1 IgR, JI1B33, exhibited some T cell activation in the presence of 1 nM Rituximab relative to 1 nM Trastuzumab. The anti-Fc HP6017 HzUCHT1 IgR, JIB34, exhibited substantial T cell activation at both 1 nM and 0.1 nM IgR with Rituximab relative to Trastuzumab indicating use of a human pan-subclass (binds human IgG1, IgG2, IgG3 and IgG4) anti-Fc antibody in a Fab-like format fused to an immune cell surface protein binding antibody is a tractable architecture to bridge target-cell-bound antibodies to immune cell receptors and avoid immune cell activity from non-specific (unbound) antibodies. The AVIG HzUCHT1 fusion, JIB35, exhibited some T cell activation 1 nM and 0.1 nM Rituximab relative to Trastuzumab. When compared to the wild type FcγR3A-HzUCHT1 fusion, JIB1, the FcγR3A_mut1-HzUCHT1 JIB36 and FcγR2A_mut-HzUCHT1 JIB37 IgRs exhibited increased T cell activation potency at both 1 and 0.1 nM IgR with Rituximab without increasing signal from Trastuzumab alone indicating an Fc receptor comprising one or multiple mutations can be used can to enhance the potency of an IgR. In contrast, the FcγR3A_mut2-HzUCHT1 JIB37 IgR had a reduced level of T cell activation at both 1 nM and 0.1 nM with Rituximab. The HzUCHT1 Fab-like molecule with one FcγR3A fused to the N-terminus of its VH and one FcγR3A fused to its VL comprising a total of two FcγR3A per JIB40 IgR exhibited lower T cell activation than the single FcγR3A comprising FcγR3A-ScFv-HzUCHT1 JIB1 IgR at both concentrations in the presence of Rituximab.

### Example 20. Evaluation of IgRs' Ability to Bridge Cancer Cells to Immune Cells in the Presence of Tumor Specific IgG and High Concentrations of Nonspecific IgG

Rituximab is an anti-CD20 IgG1 that will specifically bind Raji cells but not T cells. Trastuzumab is an anti-HER2 IgG1 that will not specifically bind Raji cells or T cells. IgRs are capable of binding different immunoglobulins with the same IgR-binding epitopes with the same affinity in liquid phase when they are not bound to their targets regardless of their target specificity. Therefore, when not bound to their targets, Rituximab and Trastuzumab can compete for the same IgR immunoglobulin binding domains. JIB4, JI1B20, JIB34 and JIB37 IgRs at 100, 10 and 1 nM were evaluated for their ability to bridge CD20+ Raji B cells with T cells and mediate T cell activation, in the presence of 100, 10 and 1 nM Rituximab with or without 1 mg/mL Trastuzumab (anti-HER2, will not specifically bind Raji cells or T cells) to mimic the high concentrations of potentially IgR-competitive, non-target specific immunoglobulin present in vivo or with 1 mg/mL Trastuzumab without Rituximab to evaluate non-specific T cell activation due to IgRs in the presence of non-specific immunoglobulin at high concentration alone. Rituximab alone at 100, 10, and 1 nM, Trastuzumab alone at 1 mg/mL were run as negative controls and Mosunetuzumab alone as a positive control using the bioassay method described in Example 8.

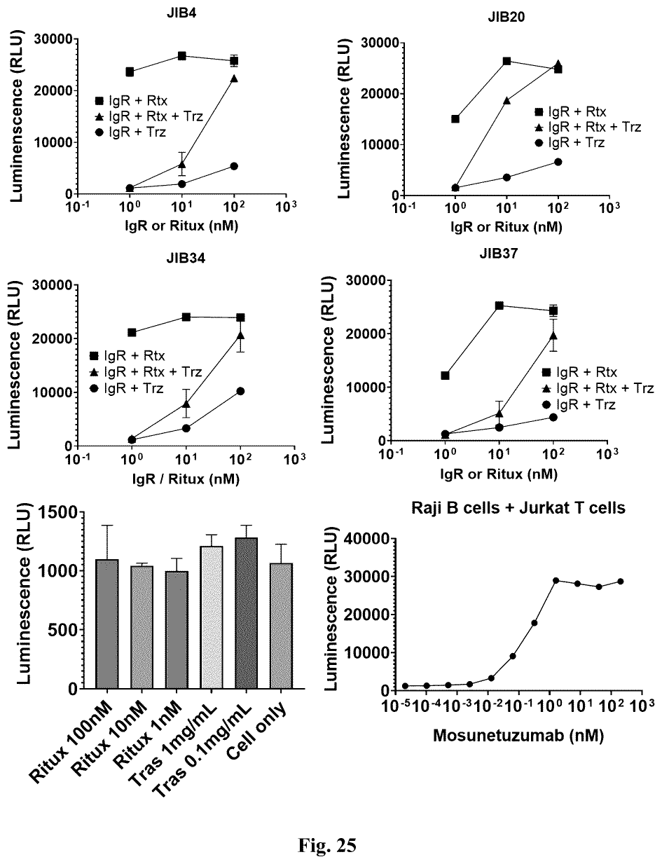

FIG. 25 shows that the αFc-VHH/ScFv-HzUCHT1 JIB4 and anti-Fc H1P6017-HzUCHT1 JIB34 IgRs have the greatest potency with Rituximab in the absence of 1 mg/mL Trastuzumab by maintaining high signal at between the 10 nM and 1 nM IgR and Rituximab conditions however all of the IgRs have substantially greater signal at all of the IgR and Rituximab conditions relative to the IgR and Trastuzumab conditions. Surprisingly, despite the potential for competition between Rituximab and Trastuzumab for an IgR's IgG-binding domains, all of the IgRs were capable of mediating T cell activation in the presence of Rituximab and 1 mg/mL Trastuzumab that was substantially greater than the activation observed in presence of IgR with Trastuzumab alone indicating IgRs can take advantage of avidity due to the multivalent availability of immunoglobulins when they are bound to targets present on cells or present in immune complexes. Additionally, at the 100 nM condition, all of the IgRs were capable of mediating T cell activation in the presence of Rituximab and 1 mg/mL Trastuzumab that was not substantially less than in the presence of Rituximab alone. We were also surprised to observe at the 10 nM condition that JIB20, comprising two immunoglobulin binding domains per IgR molecule, was capable of mediating T cell activation in the presence of Rituximab and 1 mg/mL Trastuzumab that was not substantially less than in the presence of Rituximab alone and was greater that of one immunoglobulin binding domain comprising JIB4, JIB34 and JIB37 at the same conditions despite JIB20 having a lower potency than JIB4 and JIB34 and a similar potency to the high affinity JIB37 in the absence of 1 mg/mL Trastuzumab. These results indicate that IgRs are capable of mediating biologically functional synapse formation and immune cell activity by bridging target-bound immunoglobulins to immune cell surface proteins even in the presence of high concentrations of non-target specific immunoglobulins that are capable of binding the same immunoglobulin binding domain of an IgR, similar to the conditions in vivo. These results also indicate that having more than one IgG-binding domain per IgR molecule can enhance an IgRs potency and ability to bridge target-bound immunoglobulins to immune cell surface proteins in the presence of high concentrations of non-specific immunoglobulins that are capable of binding the same immunoglobulin binding domains of an IgR.

### Example 21. Expression, Purification and Evaluation of αCD3 IgRs with Multivalent Binding to IgG Via Multiple Anti-IgG, Anti-Fc or FcγR Domains

IgG and Fc-binding heterologous polypeptides and heteromultimeric proteins were expressed and purified as described in Example 1 and Example 11.

The VH of TR66 (SEQ ID 29) was fused to the buried disulfide CH1 heavy chain (SEQ ID 100) with the extracellular domain of Fc receptor FcγR3AV-S197P (SEQ ID NO: 23) fused to the C-terminus of the constant heavy chain via a (G4S)₃ and his tag (SEQ ID NO: 21) forming CH1bDS-TR66-FcγR3AV-S197P-his (SEQ ID NO: 304) and the VL of TR66 (SEQ ID NO: 30) was fused to the kappa light chain (kappabDS, SEQ ID NO: 101) with the extracellular domain of Fc receptor FcγR3AV-S197P (SEQ ID NO: 23) fused to the C-terminus of the constant light chain via a (G4S)₃ (kappabDS-HzUCHT1-FcγR3AV-S197P, SEQ ID NO: 305) and heavy chain as depicted in FIG. 5B to form JIB44 (SEQ ID NO: 304 and SEQ ID NO: 305). Alternatively, FcγR3AV-S197P was fused to the N-terminus of both the TR66 VH and VL (JIB46: SEQ ID NO: 306, SEQ ID NO: 307) for 2 Fc-binders per as shown in FIG. 5K and additionally to the C-terminus of the constant heavy chain (JIB45: SEQ ID NO: 308, SEQ ID NO: 307) for 3 Fc-binders per molecule as shown in FIG. 5L.

CH1bDS was fused to C233S-Fc-LALAPA-hole (SEQ ID NO: 40) excluding the redundant “EPKSSDKTHT” on SEQ 40 to form CH1bDS-Fc-LALAPA-hole (SEQ 309). FcγR3AV-S197P (SEQ 23) was fused to the N-terminus of SEQ 309 and kappabDS via (G4S)₃ linkers forming FcγR3AV-S197P-CH1bDS-Fc-LALAPA-hole (SEQ ID NO: 310) and FcγR3AV-S197P-kappabDS (SEQ ID NO: 311). The IgR comprising 2 FcγR per molecule, JIB47, was produced by expressing all 4 chains: SEQ 310, SEQ311, SEQ 44, SEQ 47 as shown in FIG. 4E.

The VH of HP6017 was fused to the N-terminus and the C-terminus of a spacer derived from the CH1 domain, SEQ ID NO: 312, forming HP6017-2xVH (SEQ 313). The VH of TR66 was fused to the C-terminus of HP6017-2xVH via the SEQ 311 linker and was attached to the N-terminus of CH1bDS forming HP6017-2xVH-TR66-VH-CH1bDS-his (SEQ ID NO: 314). The VL of HP6017 was fused to the N-terminus and the C-terminus of a spacer derived from the kappa domain, SEQ ID NO: 315, forming HP6017-2xVL (SEQ 316). The VL of TR66 was fused to the C-terminus of HP6017-2xVL via the SEQ 314 linker and was attached to the N-terminus of kappabDS forming HP6017-2xVL-TR66-VL-kappabDS (SEQ ID NO: 317). The IgR, JIB48, comprising two Fc-binding domains per molecule was produced by expressing both chains (SEQ ID NO: 314, SEQ ID NO: 317) as shown in FIG. 5M. Additionally, HP6017-2xVH was fused to the N-terminus of CH1bDS-his forming HP6017-2xVH-CH1bDS-his (SEQ ID NO: 318) and HP6017-2xVL was fused to the N-terminus of kappabDS with TR66-ScFv fused to the C-terminus via a (G4S)₃ linker forming HP6017-2xVL-kappabDS-TR66-ScFv (SEQ ID NO: 319). The IgR JIB49, which also comprises two Fc-binding domains per molecule, was produced by expressing SEQ 318 and SEQ ID NO: 319 as shown in FIG. 5I (without the half-life extension domain). Additionally, TR66-VH fused to CH1bDS and TR66-VL fused to kappabDS and both had (G4S)₃ linker on the C-terminus followed by 1P6017-ScFv in the VL-VH orientation with a (G4S)₃ linker between with a his tag on the C-terminus of the heavy chain forming SEQ ID NO: 320 and SEQ ID NO: 321 which were expressed to form JIB50 as shown in FIG. 5B.

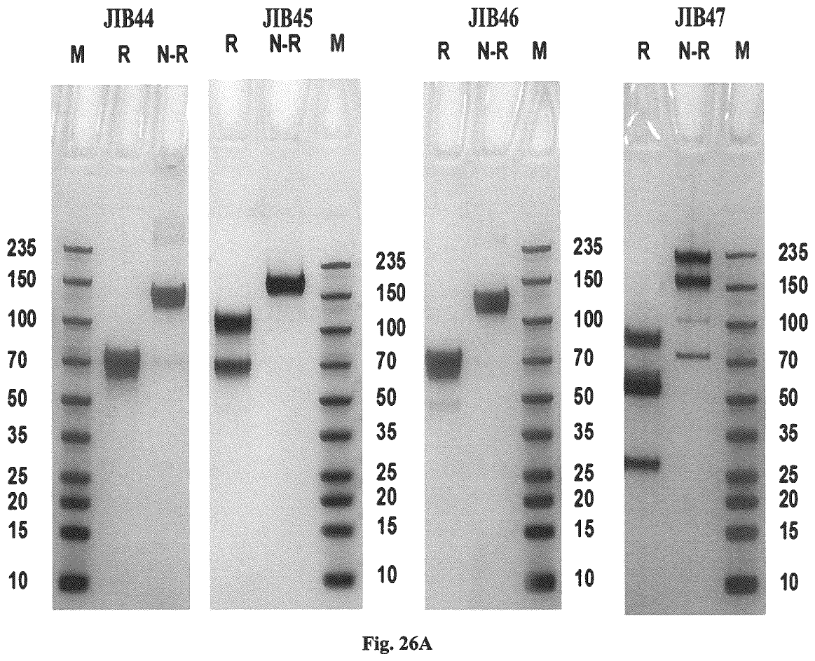

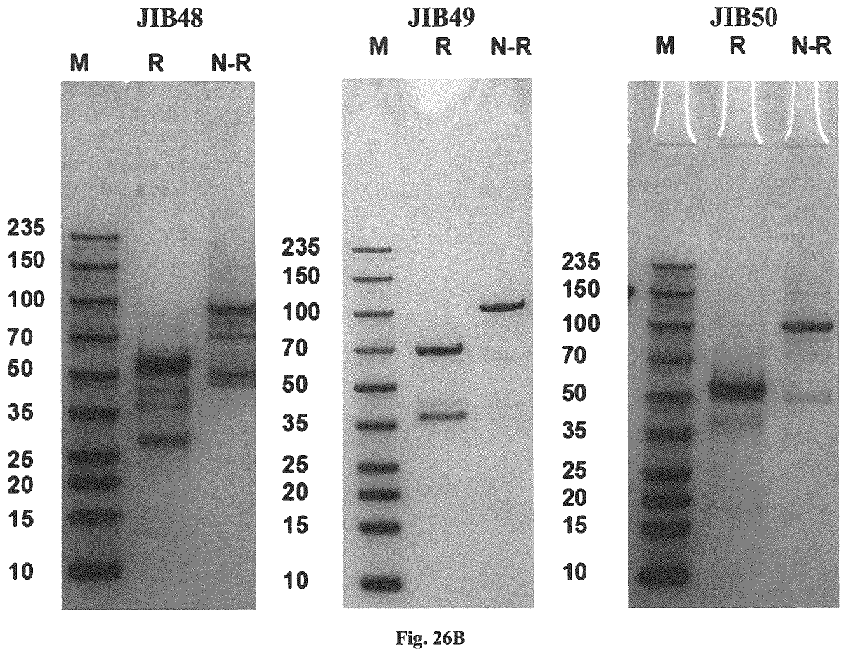

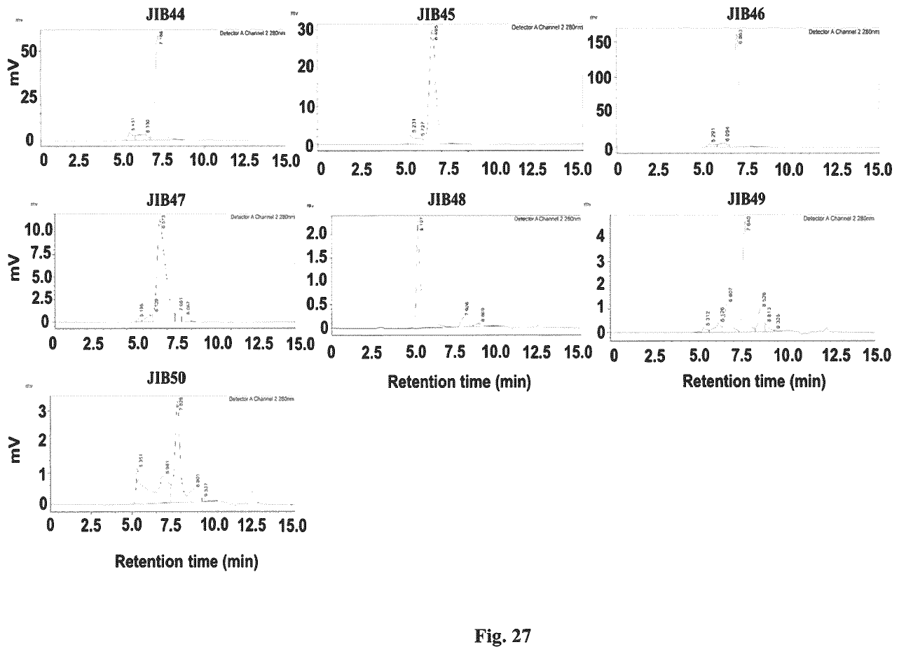

Table 10, FIG. 26A, FIGS. 26B and 27 show that all of the IgRs were well expressed with high purity with exception of JIB48, JIB49 and JIB50 with 8.8%, 54.7% and 43.6% monomer by SEC. JIB44, which is similar to the high performing JIB20 Fab-like design with a total of two FcR per IgR but used a buried interchain disulfide strategy versus an interchain disulfide knockout strategy, shows similarly high purity with 84.7% monomer versus 84.9%, respectively, indicating high purity is achievable for this Fab-like architecture regardless of whether the buried disulfide or disulfide knockout strategy is used. The consistently low purity of HP6017 in different protein architectures and with different CD3 binding clones indicated VH and VL sequence optimization was warranted to try to improve its biophysical properties. Despite the moderate purity of JIB49 and JIB50, the ability to express a dual VH-VH and VL-VL Fab like structure with an ScFv off a constant domain (JIB49) or a Fab-like structure with an ScFv off the C-terminus of each constant domain, and both with a buried interchain disulfide strategy, indicates these are tractable architectures for IgRs. The high purity of JIB47 with two Fc-binding FcγR domains and full length Fc indicates the LALAPA mutations are sufficient to knockout self-binding and aggregation, consistent with the results for JIB6. Additionally, the use of a CH1 and constant kappa chain pair with a buried interchain disulfide strategy in combination with a second CH1 and constant lambda chain pair with a natural interchain disulfide strategy did not to affect the purity of the IgR molecule implying proper heavy chain and light chain pairing.

TABLE 10
Protein Expression and % Monomer by
SEC for IgR produced in Example 21.

| Protein | CHO (mg/L) | SEC % Monomer |
|---|---|---|
| JIB44 | 90 | 84.7% |
| JIB45 | 152 | 89.5% |
| JIB46 | 180 | 92.0% |
| JIB47 | 167 | 87.2% |
| JIB48 | 8 | 8.8% |
| JIB49 | 11 | 54.7% |
| JIB50 | 23 | 43.6% |

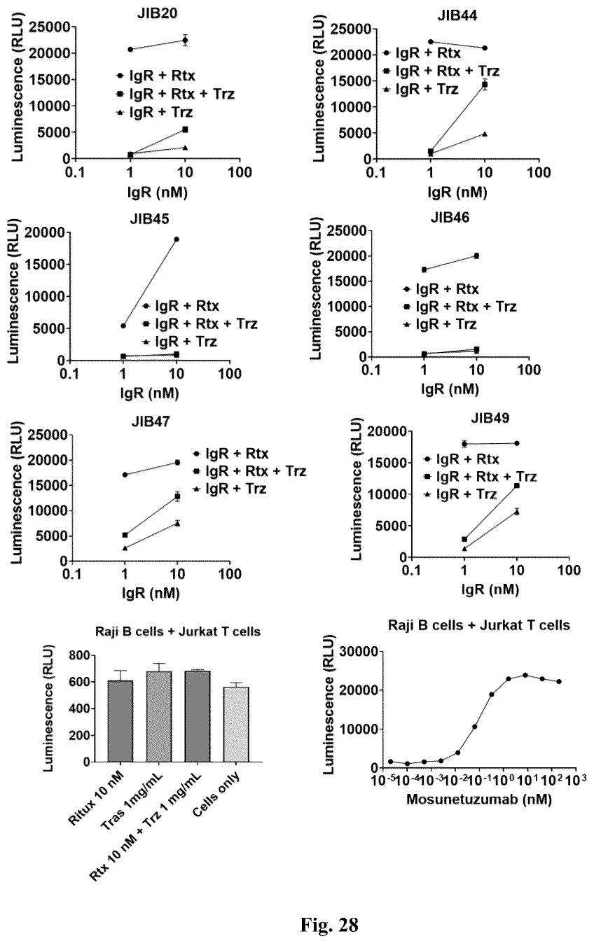

Next, the IgRs were evaluated at 10 nM and 1 nM for their ability to bridge CD20+ Raji B cells with T cells and mediate T cell activation, in the presence of 10 nM Rituximab with or without 1 mg/mL Trastuzumab or with 1 mg/mL Trastuzumab without Rituximab. Rituximab alone at 10 nM, Trastuzumab alone at 1 mg/mL and cells alone were run as negative controls and Mosunetuzumab alone as a positive control using the bioassay method described in Example 8 and Example 20. FIG. 28 shows that all of the IgRs were capable of mediating T cell activation in the presence of Rituximab alone that was substantially greater than IgRs in the presence of 1 mg/mL Trastuzumab alone. Additionally, JIB20, J1B44, JIB47 and JIB49 were capable of mediating T cell activation in the presence of Rituximab and 1 mg/mL Trastuzumab that was substantially greater than in the presence of 1 mg/mL Trastuzumab alone and was not substantially less than in the presence of Rituximab alone for the 10 nM condition. In the presence of Rituximab, with or without 1 mg/mL Trastuzumab, TR66 Fab with FcR off both the C-terminus of its light chain and heavy chain (JIB44) was more potent than a similar design using HzUCHT1 (JIB20) at 0.1 nM IgR and more potent than TR66 with FcR on the N-terminus of both the light chain and heavy chain (JIB46) and additionally off the C-terminus of the heavy chain (JIB45) at both 1 and 0.1 nM IgR. While this result indicates selection of the immune cell surface receptor binding clone can affect the potency of an IgR, it also shows the IgR Fab-like architecture with an immunoglobulin binding domain off the C-terminus of both the heavy and light chain consistently shows high potency even in the presence of competitive non-specific immunoglobulin. It also shows that having FcR off the C-terminus of the heavy and light chain of the Fab-like IgR can be superior over the N-termini of the VH and VL regions even when a third Fc-binder per molecule is fused to the C-terminus. The dual Fc-binding Fc fusion with SP34 (JIB47) and dual HP6017-VH-VH/VL-VL Fab with TR66-ScFv (JIB49) both exhibited potent T cell activation in the presence of Rituximab with or without 1 mg/mL Trastuzumab indicating that two Fc-binding FcγR or two Fc-binding antibody domains are both tractable compositions to produce target cell specific activation of immune cells while overcoming potential competition from high concentration of non-specific IgG. Additionally, JIB47 comprised one light chain/heavy chain pair using CH1bDS and kappabDS and a second light chain/heavy chain pair comprising the WT CH1 and lambda sequences indicating that having a differential in interchain disulfide status between two constant light chain and heavy chain pairs is a tractable means to enable intended constant light chain and heavy chain pairing to maintain specific and intended target binding for a multispecific protein.

### Example 22. Expression, Purification and Evaluation of αCD3 and αCD28 IgRs with Multivalent Binding to IgG Via Multiple Anti-IgG, Anti-Fc or FcγR Domains

IgG and Fc-binding heterologous polypeptides and heteromultimeric proteins were expressed and purified as described in Example 1 and Example 11.

TR66 Fab with the buried disulfide had two different anti-Fc VHHs (anti-Fc VHH2: SEQ ID NO: 322; anti-Fc VHH3, SEQ ID NO: 323), the extracellular domain of mouse FcγR3 and FcγR2b (SEC ID 324, SEQ ID NO: 325) fused to the C-terminus of it's heavy chain with C-terminal his tag and light chain and co-expressed to form JIB51 (SEQ ID NO: 326, SEQ ID NO: 327), JIB52 (SEQ ID NO: 328, SEQ ID NO: 329), JIB53 (SEQ ID NO: 330, SEQ ID NO: 331), JIB54 (SEQ ID NO: 332, SEQ ID NO: 333) as depicted in FIG. 5B. Additionally, anti-Fc VHH2 was fused with VHH2 or VHH3 was fused with VHH3 and both were fused with TR66 ScFv form JIB55 (SEQ ID NO: 334) and JIB56 (SEQ ID NO: 335) as depicted in FIG. 2C.

To improve the biophysical properties of HP6017, such as increasing % Monomer by SEC after single step purification, three different humanization strategies were performed. HzHP6017A (VH SEQ ID NO: 336, VL SEQ ID NO: 337) was designed by grafting the CDRs according to Kabat (Kabat, 1992) of HP6017 onto the top hit human germlines (IGHV1-46*01 and IGKV1-33*01) in the IMGT database (https://www.imgt.org/3Dstructure-DB/cgi/DomainGapAlign.cgi) and back-mutated to the mouse HP6017 wild type sequence in locations critical to IgG packing (Chothia et al., 1985), with known risk factors (Studnicka et al., 1994)(Foote & Winter, 1992). HzHP6017B (VH SEQ ID NO: 338, VL SEQ ID NO: 339) was designed by grafting the CDRs according to Kabat into the IGHV1-2*02 and IGKV1-12*01 human germlines in the IMGT database with VH framework mutations R93 S and D97E and VL framework mutation V11L according to IMGT numbering. HzHP6017C (VH SEQ ID NO: 340, VL SEQ ID NO: 341) was designed by using the BioPhi method previously described (Prihoda et al., 2022). Table 11 displays the % identity to the top hit human germline gene and allele in the IMGT database and highlights that the humanization method for HzHP6017B-VH/VL sequences produced the highest % identity to human germline sequences. The humanized VH and VL pairs were then fused to the CH1bDS and kappabDS sequences with HzUCHT1 ScFv off the C-terminus of the light chain via a (G4S)3 linker like JIB34 generating JIB57 (SEQ ID NO: 342, SEQ ID NO: 343), JIB58 (SEQ ID NO: 344, SEQ ID NO: 345) and JIB59 (SEQ ID NO: 346, SEQ ID NO: 347) as shown in FIG. 5C.

TABLE 11
% Identity of top hit human germline
sequences from IMGT database.

| Variable Chain | IMGT gene and allele | % Identity |
|---|---|---|
| HP6017 VH | IGHV1-46*01 | 69.4% |
| HP6017 VL | IGKV1-33*01 | 67.4% |
| HzHP6017A VH | IGHV1-46*01 | 75.5% |
| HzHP6017A VL | IGKV1-33*01 | 72.6% |
| HzHP6017B VH | IGHV1-46*01 | 88.8% |
| HzHP6017B VL | IGKV1-39*01 | 84.2% |
| HzHP6017C VH | IGHV1-46*01 | 79.6% |
| HzHP6017C VL | IGKV1-39*01 | 81.1% |

A humanized version of VHH-CH128, HzVHH-CH128 (SEQ ID NO: 348), was designed by grafting the CDRs according to Kabat into the IGHV3-64*04 human germline sequence with mutations S24A, V42F, K48Q, Y52A and S54A according to IMGT numbering. VHH-CH128 was fused to another VHH-CH128 via a (G4S)3 linker and then fused to HzUCHT1-ScFv generating JIB60, VHH-CH128-VHH-CH128-HzUCHT1-ScFv-his (SEQ ID NO: 349), as depicted in FIG. 2C.

Two tandem extracellular domains of mouse FcγRIV were fused to HzUCHT1-ScFv (G4S)₃ linkers generating JIB61, mFcγRIV-mFcγRIV-HzUCHT1-ScFv-his (SEQ ID NO: 350), as depicted in FIG. 2C. αFc-10-VHH was fused to the N-terminus HzUCHT1-ScFv via a (G4S)₃ linker followed by the anti-HSA-VHH1 (SEQ ID NO: 351) or αMSA-VHH forming JIB62, αFc-10-VHH-HzUCHT1-ScFv-anti-HSA-VHH1 (SEQ ID NO: 352), and JIB63, αFc-10-VHH-HzUCHT1-ScFv-αMSA-VHH (SEQ ID NO: 353), as depicted in FIG. 3A.

To explore a different architecture and anti-CD3 clone with the anti-IgG 17F12 clone, TR66 Fab using C4CH1-bDS and kappabDS was fused with 17F12QUAD-ScFv off the C-terminus of the light chain and heavy chain via (G4S)3 linkers generating JIB64 (SEQ ID NO: 354, SEQ ID NO: 355) as depicted in FIG. 5B.

Anti-CD28 IgR proteins were designed by fusing the extracellular domain of human FcγR2A (SEQ ID NO: 9) to the C-terminus of CH1bDS and kappabDS forming CH1bDS-FcγR2A-his (SEQ ID NO: 356) and kappabDS-FcγR2A (SEQ ID NO: 357) comprising the VH and VL pairs of 2E12 (SEQ ID NO: 205, SEQ ID NO: 207), TGN1412 (SEQ ID NO: 210, SEQ ID NO: 212), 9.3 (SEQ ID NO: 215, SEQ ID NO: 217), 8GA8 (8GA8-VH SEQ ID NO: 358, 8GA8-VL SEQ ID NO: 359) 9D7 (9D7-VH SEQ ID NO: 360, 9D7-VL SEQ ID NO: 361) and TN228 (TN228-VH SEQ ID NO: 362, TN228-VL SEQ ID NO: 363) generating JIB67 (SEQ ID NO: 364, SEQ ID NO: 365), JIB68 (SEQ ID NO: 366, SEQ ID NO: 367), JIB69 (SEQ ID NO: 368, SEQ ID NO: 369), JIB70 (SEQ ID NO: 370, SEQ ID NO: 371), JIB71 (SEQ ID NO: 372, SEQ ID NO: 373) and JIB72 (SEQ ID NO: 374, SEQ ID NO: 375) respectively, as depicted in FIG. 5B.

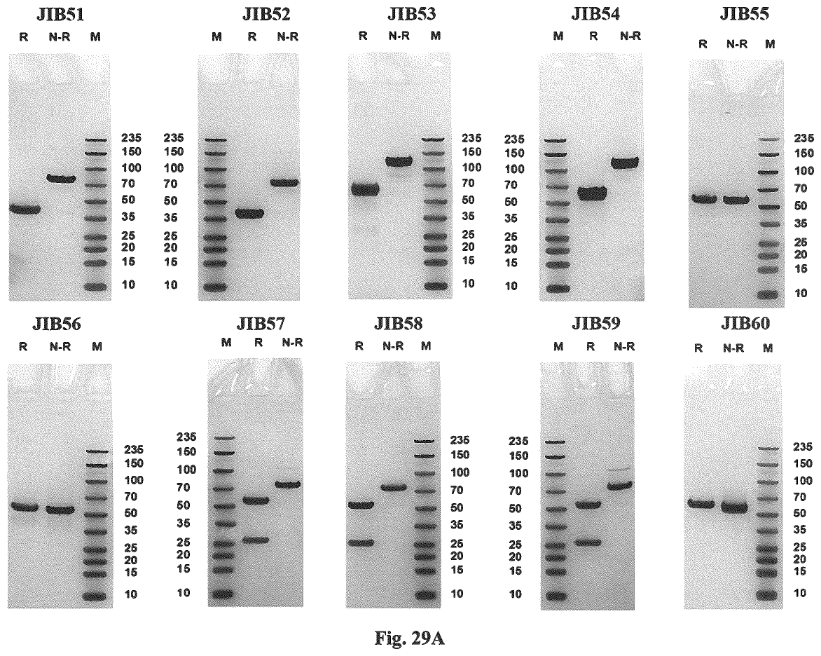

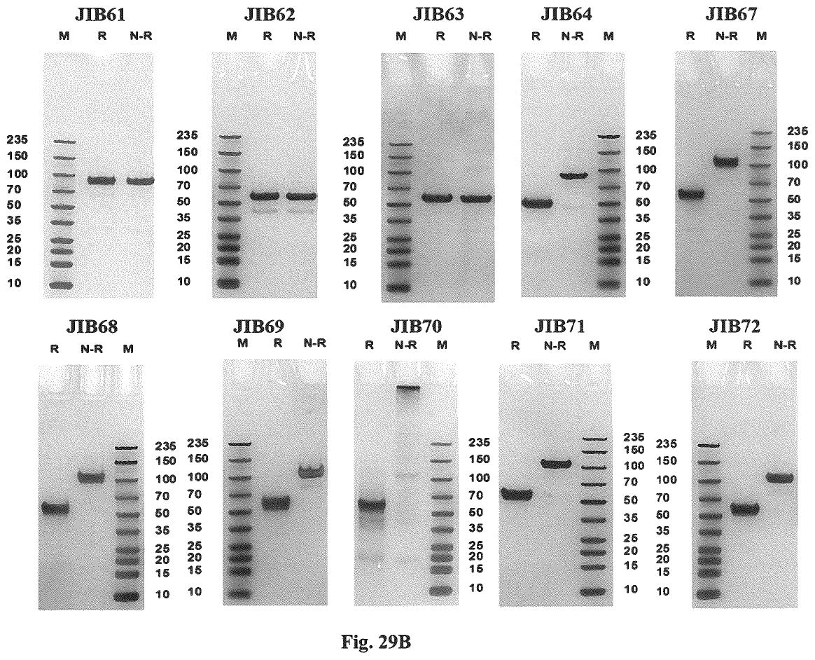

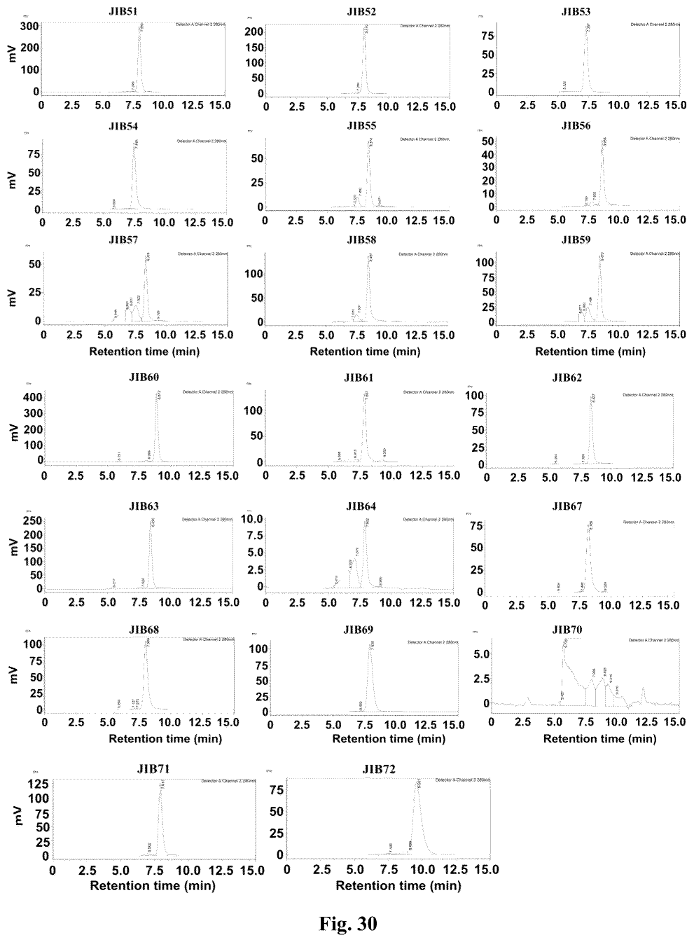

Table 12, FIG. 29A, FIG. 29B, and FIG. 30 show that all of the IgRs were well expressed with high purity after single step purification with the exception of JIB57, JIB59, JIB64 and JIB70 with 46.9%, 57.5%, 43.4% and 14.7% monomer by SEC, respectively. In contrast to the wild type HP6017-VH/VL for JIB34, humanized HP6017A-VH/VL for JIB57 and humanized HP6017C-VH/VL for JIB59, the HP6017B-VH/VL humanization strategy for JIB58 produced an IgR of the highest purity amongst the four with 79.4% monomer by SEC versus 51.8% for JIB34, 46.9% for JIB57, 57.5% for JIB59 indicating the HP6017B humanization strategy had the additional benefit of improving the biophysical properties of the IgR. JIB51, JIB52, JIB53 and JIB54 IgRs with Fab-like structures and two immunoglobulin domains, one off of both the heavy and light chain, show that fused VHH domains do not mispair with the VL of the Fab and that the use of mouse Fc receptors does also not have issues with expression or purity. Similarly, JIB55, JIB56, JIB60 and JIB61 with two tandem anti-immunoglobulin binding domains including anti-Fc and anti-CH1 VHHs or mouse Fc receptors fused in a head to tail orientation along with a C-terminal anti-CD3 domain did not have issues with expression or purity. The anti-CD28 JIB67, JIB68, JIB69, JIB70, JIB71 and JIB72 all have the same Fab-like structure similar to that of anti-CD3 JIB20, JIB44, JIB51, JIB52, JIB53 and JIB52 but with two FcγR2A fused to the heavy chain and light chain versus immunoglobulin binding VHHs, FcγR3A or mouse FcγR. The high expression and purity of all of these constructs (with exception of JIB70) indicates this particular IgR architecture can be produced at generally high expression and purity from single step purification if the starting components are amenable. In the case of JIB70, the low purity and expression is caused by the VH/VL pair used in the Fab-like scaffold and not the immunoglobulin binding domains incorporated.

TABLE 12
Protein Expression and % Monomer by
SEC for IgR produced in Example 22.

| Protein | CHO (mg/L) | SEC % Monomer |
|---|---|---|
| JIB51 | 293 | 98.0% |
| JIB52 | 224 | 95.0% |
| JIB53 | 88 | 97.1% |
| JIB54 | 127 | 99.4% |
| JIB55 | 125 | 77.0% |
| JIB56 | 94 | 92.0% |
| JIB57 | 150 | 46.9% |
| JIB58 | 225 | 79.4% |
| JIB59 | 215 | 57.5% |
| JIB60 | 358 | 96.6% |
| JIB61 | 130 | 83.3% |
| JIB62 | 119 | 99.0% |
| JIB63 | 307 | 98.6% |
| JIB64 | 35 | 43.4% |
| JIB67 | 67 | 99.4% |
| JIB68 | 132 | 98.9% |
| JIB69 | 176 | 98.7% |
| JIB70 | 8 | 14.7% |
| JIB71 | 84 | 96.6% |
| JIB72 | 217 | 95.4% |

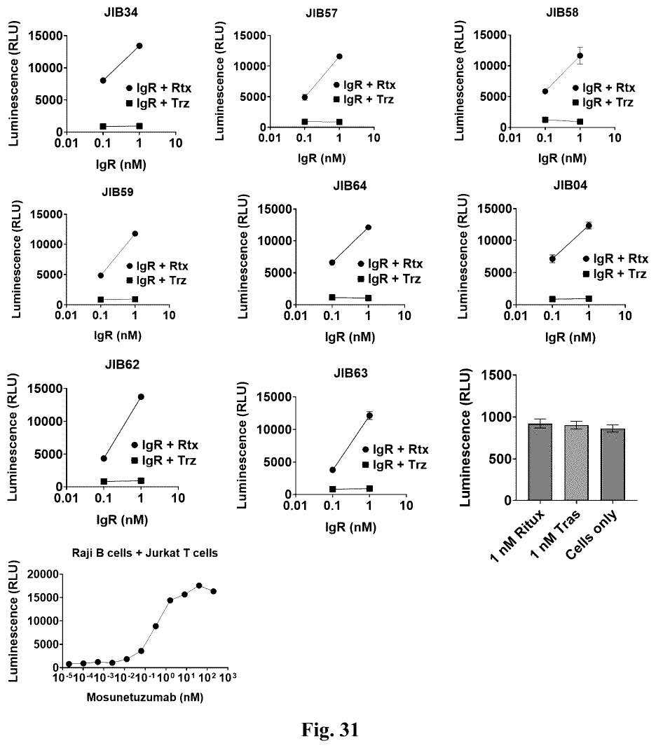

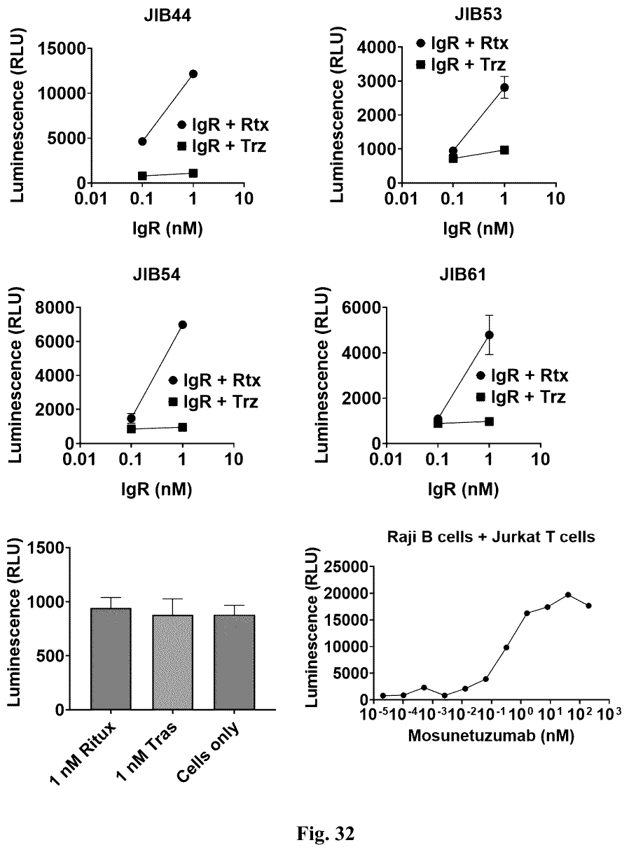

Next, the anti-CD3 IgRs JIB34, JIB57, JIB58, JIB59, JIB64, JIB4, JIB62, JIB63, JI1B44, JIB53, JIB54 and JIB61 were evaluated at 1 nM and 0.1 nM for their ability to bridge CD20+ Raji B cells with Jurkat T cells and mediate T cell activation, in the presence of 1 nM Rituximab to evaluate target cell specific activation or with 1 nM Trastuzumab to evaluate nonspecific activation. Rituximab alone at 1 nM, Trastuzumab alone at 1 nM and cells alone were run as negative controls and Mosunetuzumab alone as a positive control using the bioassay method described in Example 8. FIG. 31 shows that the humanized anti-Fc HP6017 IgRs (JIB57, JI1B58, JIB59) have comparable T cell activation to the mouse wild type HP6017 IgR at both 1 and 0.1 nM Rituximab and the activation substantially greater than the IgRs in the presence of Trastuzumab indicating the humanization methods minimally reduced the potency of the antibodies and did not increase non-specific activation. Moreover, in addition to JIB58 having the highest % identity to its top hit human germline sequence and the highest % monomer by SEC, it also had the highest potency of the three humanized HP6017 IgRs evaluated. JIB64, the TR66 Fab comprising two IgG binders per molecule with 17F12QUAD ScFv off both the light chain and heavy chain had comparable potency at both 1 and 0.1 nM Rituximab to the WT and humanized HP6017 IgRs (JIB20, JIB57, JIB58 and JIB59) which is in contrast with the JIB33 17F12QUAD Fab-like IgR which gave little T cell activation over background in Example 19. This indicates the C4CH1 P126S and K213E mutations, which ablated self-binding, and 17F12QUAD VL mutations, to improve expression and stability, enabled high purity for a 17F12 Fab while maintaining target binding for the 17F12-VH and 17F12QUAD-VL pair and that a Fab-like structure should be pursued with an alternative immune cell surface protein binding clone. JIB62 and JIB63 which are similar to JIB4 with anti-Fc-10-VHH fused to HzUCHT1 but with anti-HSA or anti-MSA fused to the C-terminus both had comparable potency with 1 nM Rituximab and only a small reduction in T cell activation at 0.1 nM Rituximab without increasing the activation in the presence of Trastuzumab indicating the presence of the serum albumin binders on the C-terminus of the IgR is minimally interfering with mediating IgG and immune cell target binding and function and does not increase nonspecific immune cell activity. In FIG. 32, comparing TR66 Fabs with two FcγR per IgR comprising human FcγR3A (JIB44), mFcγR3 (JIB53) or mFcγR2b (JIB54) shows that an IgRs with human FcγR3A has the highest potency at both 1 and 0.1 nM Rituximab and the IgR potency between the three is consistent with known monovalent affinity (K_{D}) rankings for human IgG1 where FcγR3A (68 nM)>mFcγR2b (1100 nM)>mFcγR3 (9300 nM)(Dekkers et al., 2017; Patel et al., 2019). This data indicates that IgRs can function even when comprising low affinity IgG binders in the approximately 10 M monovalent K_{D} range. The IgR JIB61 with two mFcγRIV in tandem with HzUCHT1-ScFv had a potency between that of JIB53 and JIB54.

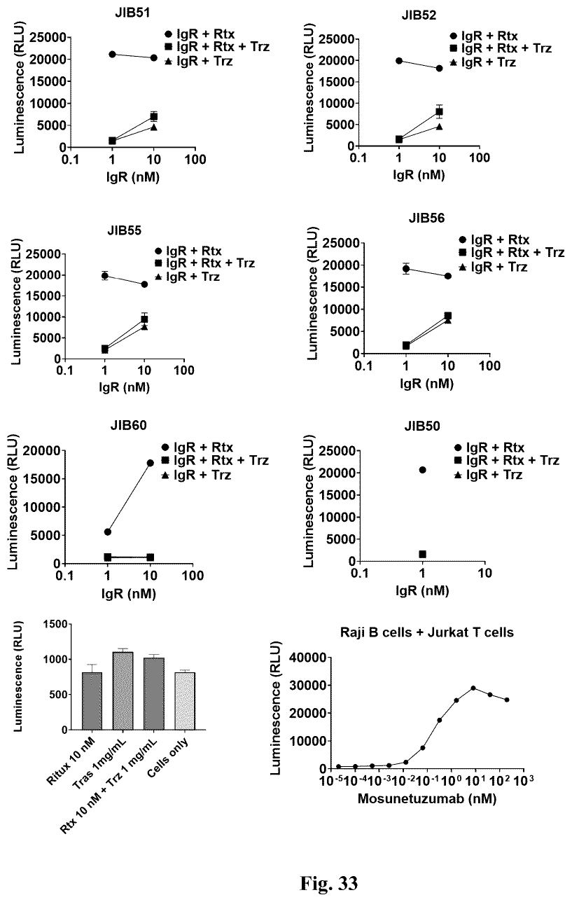

The anti-CD3 IgRs JIB51, JIB52, JIB55, JIB56, JIB60, JIB65 and JIB50 were evaluated at 10 nM and 1 nM for their ability to bridge CD20+ Raji B cells with Jurkat T cells and mediate T cell activation, in the presence of 10 nM Rituximab with or without 1 mg/mL Trastuzumab or with 1 mg/mL Trastuzumab without Rituximab. Rituximab alone at 10 nM, Trastuzumab alone at 1 mg/mL and cells alone were run as negative controls and Mosunetuzumab alone as a positive control using the bioassay method described in Example 8 and Example 20. In FIG. 33, comparing TR66 Fab-like designs with two anti-Fc VHHs per IgR (JIB51, JIB52) off the C-terminus of the heavy and light chains with the same two anti-Fc VHHs in a head-to-tail arrangement in tandem with TR66-ScFv (JIB55, JIB56) shows that both formats mediate T cell activation in the presence of 10 or 1 nM Rituximab that is substantially greater than with 1 mg/mL Trastuzumab. Interestingly, the TR66 Fab-like format appears to produce less non-specific activation than the tandem designs in the presence of 1 mg/mL Trastuzumab alone and a greater ratio in signal between IgR in the presence of Rituximab and 1 mg/mL Trastuzumab versus IgR with Trastuzumab alone. JIB60, the anti-CH1 IgR with two VHH in tandem with HzUCHT1 appears to have lower potency than the tandem anti-Fc IgRs JIB55 and JIB56 at 1 nM Rituximab. The JIB50 TR66 Fab comprising two anti-Fc HP6017-ScFv per IgR with one off the HC and one off LC shows comparable T cell activation to JIB51, JIB52 at 1 nM Rituximab in the absence or presence of 1 mg/mL.

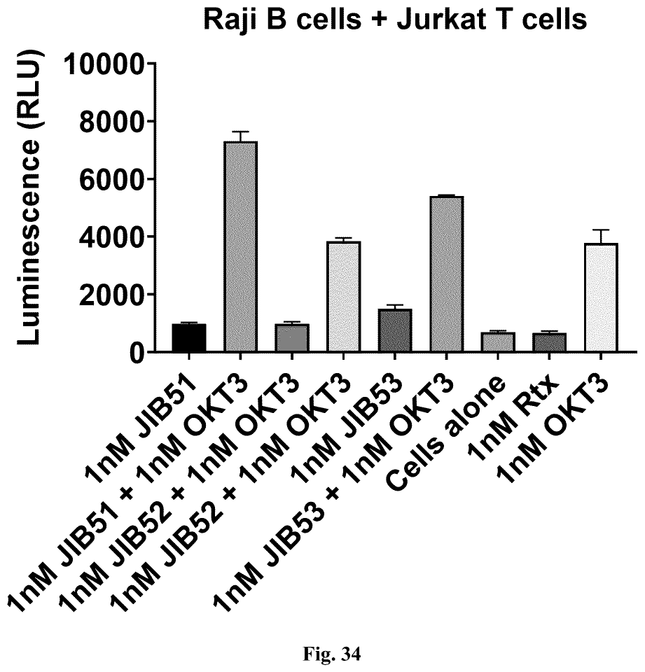

The anti-CD3 IgRs JIB51, JIB52 and JIB53 were evaluated at 1 nM for their ability to bind mouse IgG and mediate T cell activation in combination with 1 nM anti-CD3 OKT3 mIgG2a (Biolegend, 317325) relative to IgR alone at 1 nM and OKT3 alone at 1 nM. Rituximab (Rtx) alone at 1 nM was used as a negative control. Test articles were co-cultured with CD20+ Raji B cells and Jurkat-NFAT-Luc T cells and read out for luminescence using the procedures from Example 8. FIG. 34 shows that JIB51 and JIB53 mediate substantially greater activation in the presence of OKT3 relative to OKT3 alone and JIB51 does not have substantially greater activation when used alone relative to Rituximab alone or cells alone. JIB52 does not have substantially greater activation in the OKT3 relative to OKT3 alone. These results indicate JIB51 cross-reacts with mouse IgG and more so than the mouse FcγR3 comprising JIB53 IgR.

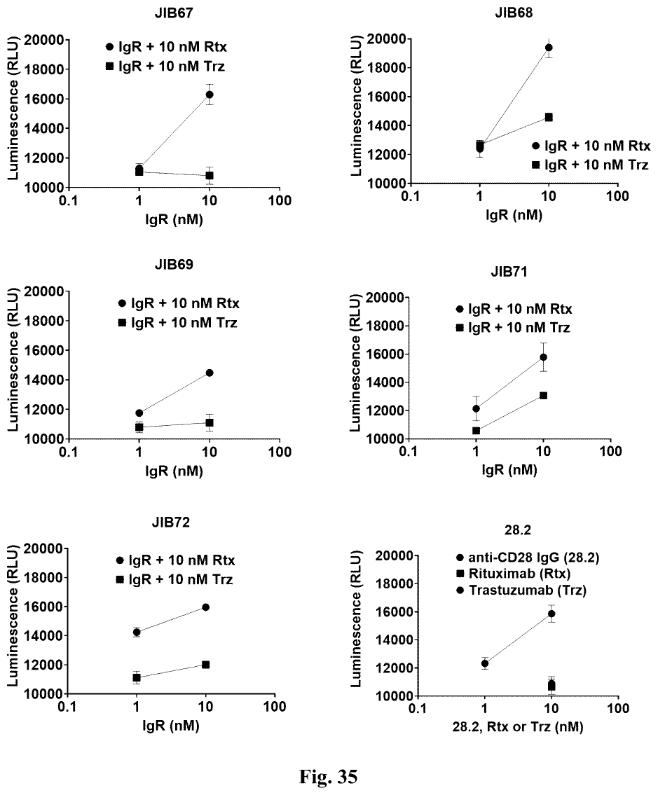

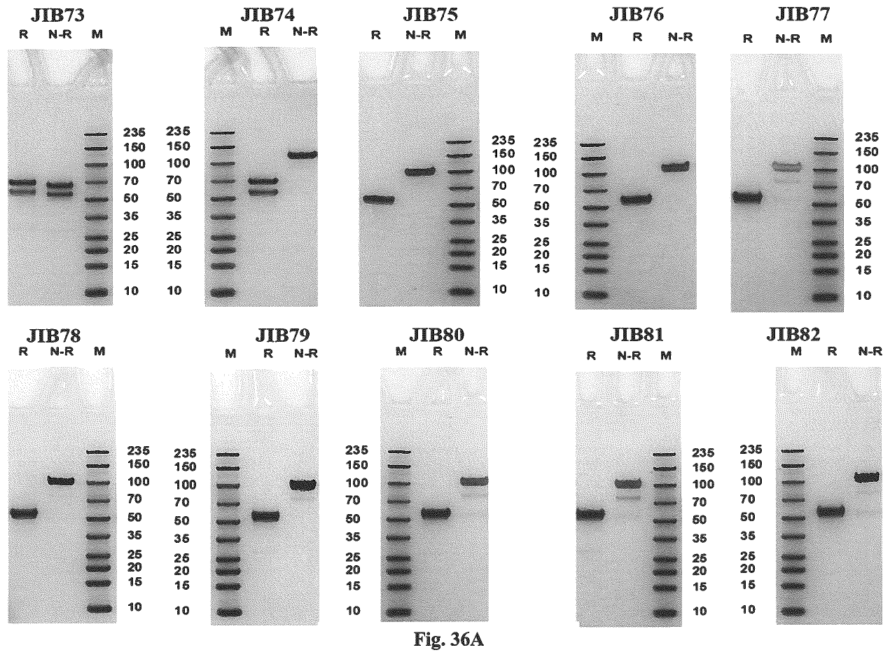

The anti-CD28 IgRs JIB67, JIB68, JIB69, JIB71 and JIB72 were evaluated using a modified version of the co-culture assay described in Example 8 where 96 well U-bottom plates were coated with 100 μl of 1 μg/mL anti-CD3 OKT3 (Biointron, B6928, Mouse IgG2a(D134G)CH1+Human IgG1 CH2+CH3-Mouse kappa) overnight at 4° C. and washed with PBS once the next day prior to the cell co-culture assay to remove any unbound OKT3 from the wells. IgRs were evaluated at 10 nM and 1 nM for their ability to bridge CD20+ Raji B cells with Jurkat T cells and mediate T cell co-stimulation (OKT3 IgG on the plate is providing signal 1), in the presence of 10 nM Rituximab to evaluate target cell specific T cell co-stimulation activity or with 10 nM Trastuzumab to evaluate nonspecific T cell co-stimulation activity. Rituximab alone at 10 nM, Trastuzumab alone at 10 nM and cells alone were run as negative controls and anti-CD28 mIgG1 clone CD28.2 (Thermo, 16-0289-85) alone at 10 nM and 1 nM was used as a positive T cell co-stimulation control. FIG. 35 shows that all of the anti-CD28 IgRs when combined with Rituximab had similar and in some cases greater potency than the anti-CD28 CD28.2 IgG. All of the IgRs had substantially greater T cell co-stimulation activity in combination with Rituximab relative to their activity in combination with the non-target cell specific Trastuzumab. JIB67, JIB69 and JIB72 exhibited activity in combination with Trastuzumab that was not substantially greater than the activity of Trastuzumab alone, indicating these IgRs have minimal non-specific T cell co-stimulation activity. In contrast, JIB68 and JIB71 exhibited some non-specific T cell co-stimulation activity when comparing them combined with Trastuzumab versus Trastuzumab alone.

### Example 23. Expression, Purification and Evaluation of αCD3 and αTCRα/β IgRs with Multivalent Binding to IgG Via Multiple Anti-IgG, Anti-Fc or FcγR Domains

IgG and Fc-binding heterologous polypeptides and heteromultimeric proteins were expressed and purified as described in Example 1 and Example 11.

SP34 Fab using CH1NoDS and hLC7NoDS was fused with FcγR2A on the C-terminus of both the constant heavy chain and light chain via (G4S)₃ linkers followed by the αHSA-VHH with a C-terminal his tag on the heavy chain to form JIB73 (SEQ ID NO: 376, SEQ ID NO: 377) as depicted in FIG. 5J. Similarly, HzUCHT1 using CH1bDS and kappabDS was fused with FcγR2A on the C-terminus of both the constant heavy chain and light chain via (G4S)₃ linkers followed by the αHSA-VHH with a C-terminal his tag on the heavy chain to form HzUCHT1-based JIB74 (SEQ ID NO: 378, SEQ ID NO: 379) as depicted in FIG. 5J. TR66, HzTR66, Hu291, huCLB-T3/4.A, HuYTH 12.5, BC3, Hz06 and αTCR BMA-031 Fab using CH1bDS and kappabDS (or hLC7bDS for HuYTH 12.5 and Hz06) was fused with FcγR2A on the C-terminus of both the constant heavy chain and light chain via (G4S)₃ linkers followed by a C-terminal his tag on the heavy chain to form TR66-based JIB75 (SEQ ID NO: 380, SEQ ID NO: 381), HzTR66-based JIB76 (SEQ ID NO: 382, SEQ ID NO: 383), Hu291-based JIB77 (SEQ ID NO: 384, SEQ ID NO: 385), huCLB-T3/4.A-based JIB78 (SEQ ID NO: 386, SEQ ID NO: 387), HuYTH 12.5-based JIB79 (SEQ ID NO: 388, SEQ ID NO: 389), BC3-based JIB80 (SEQ ID NO: 390, SEQ ID NO: 391), Hz06-based JIB81 (SEQ ID NO: 392, SEQ ID NO: 393), BMA-031-based JIB82 (SEQ ID NO: 394, SEQ ID NO: 395) as depicted in FIG. 5B. HzTR66 was generated using a method similar to that of HzHP6017B where HzTR66 VH SEQ ID NO: 396 and VL SEQ ID NO: 397 were designed by grafting the CDRs according to Kabat into the IGHV1-2*02 and IGKV1-12*01 human germlines in the IMGT database with VH framework mutations R93S and D97E and VL framework mutation V11L according to IMGT numbering.

FcγR2A was fused to the N-terminus of CH1bDS-Fc-LALAPA-hole and kappabDS via (G4S)₃ linkers forming FcγR2A-CH1bDS-Fc-LALAPA-hole (SEQ 398) and FcγR2A-kappabDS (SEQ 399). SP34-Fc-LALAPA-knob was mutated with C233S and SP34 VL was fused with hLC7NoDS to knock out the inter-chain disulfide forming SP34-NoDS-Fc-LALAPA-knob (SEQ ID NO: 400) and SP34-hLC7NoDS (SEQ ID NO: 401). HzUCHT1-VH was swapped with SP34 in SP34-Fc-LALAPA-knob to form HzUCHT1-Fc-LALAPA-knob (SEQ ID NO: 402). HzUCHT1 VL was fused with kappaNoDS for form HzUCHT1-kappaNoDS (SEQ ID NO: 403). SP34-based JIB85 and HzUCHT1-based JIB84 was produced by coexpressing FcγR2A-CH1bDS-Fc-LALAPA-hole and FcγR2A-kappabDS together with SP34-NoDS-Fc-LALAPA-knob and SP34-hLC7NoDS or HzUCHT1-Fc-LALAPA-knob and HzUCHT1-kappaNoDS as depicted in FIG. 4E. JIB86 was produced by co-expressing SP34-NoDS-Fc-LALAPA-knob and SP34-hLC7NoDS-FcγR2A (SEQ ID NO: 377) with FcγR2A fused to the N-terminus of C233S/Fc-LALAPA-hole via a (G4S)₃ linker generating FcγR2A-C233S/Fc-LALAPA-hole (SEQ ID NO: 404) as depicted in FIG. 4G. JIB87 and JIB88 was generated by fusing a second FcγR2A onto the N-terminus of FcγR2A-C233S/Fc-LALAPA-hole via a (G4S)₃ linker forming (SEQ ID NO: 405) and co-expressing with C233S/Fc-LALAPA-knob (SEQ ID NO: 406) fused to either HzUCHT1-ScFv (SEQ ID NO: 407) or SP34-ScFv (SEQ ID NO: 408) via (G4S)₃ linkers as depicted in FIG. 4C but with an additional IgBD on the N-terminus of the first IgBD.

JIB89 was generated by fusing FcγR2A followed by SP34-ScFv, FcγR2A and αHSA-VHH via (G4S)₃ linkers forming FcγR2A-SP34-ScFv-FcγR2A-αHSA-VHH (SEQ ID NO: 409) as depicted in FIG. 3B (in a rearranged order). Similarly, JIB90 was generated but using HzUCHT1-ScFv forming FcγR2A-HzUCHT1-ScFv-FcγR2A-αHSA-VHH (SEQ ID NO: 410). JIB91 and JIB92 are IgRs comprising two Fc-binding domains per molecule and have heavy chains HzHP6017B-2xVH-CH1bDS-αHSA-VHH (SEQ ID NO: 411) and light chains HzHP6017B-2xVL-kappabDS-HzUCHT1-ScFv (SEQ ID NO: 412) or HzHP6017B-2xVL-kappabDS-SP34-ScFv (SEQ ID NO: 413), respectively as depicted in FIG. 5I. JIB93 is an IgR comprising two Fc-binding domains per molecule with a heterodimeric Fc, as depicted in FIG. 4I, and was generated using 4 chains, the first light chain comprised HzHP6017B-2xVL-kappabDS (SEQ ID NO: 414), the first heavy chain comprised HzHP6017B-2xVH-CH1bDS-Fc_mut-LALAPA-hole (SEQ ID NO: 415) with additional Fc mutations N276K, L309V (EU Numbering) to ablate HzHP6017B self Fc-binding, the second light chain comprised SP34-hLC7NoDS (SEQ ID NO: 401) and the second heavy chain comprised SP34-VL-CH1NoDS-Fc_mut-LALAPA-knob (SEQ ID NO: 416) with additional Fc mutations N276K, L309V to ablate HzHP6017B self Fc-binding. JIB94 is an IgR comprising two Fc-binding domains per molecule with a heavy chain comprising SP34-VH-CH1NoDS-HzHP6017B-ScFv-his (SEQ ID NO: 417) and a light chain comprising SP34-VL-hLC7NoDS-HzHP6017B-ScFv (SEQ ID NO: 418) as depicted in FIG. 5B.

JIB95 is an IgR comprising two anti-IgG binding domains per molecule with a heavy chain Hz17F12-2xVH-C4CH1bDS-αHSA-VHH-his (SEQ ID NO: 419) where the CH1 comprises the P126S and K213E mutations used in JIB33 to ablate Hz17F12 self-binding and the VH-VH spacer comprises the P126S mutation (C4CH1spacer, SEQ ID NO: 557) and a light chain Hz17F12-2xVL-kappabDS-SP34-ScFv (SEQ ID NO: 420) as depicted in FIG. 5I (without the half-life extension domain). Hz17F12 was generated using a method similar to that of HzHP6017B where Hz17F12 VH SEQ ID NO: 421 and VL SEQ ID NO: 422 were designed by grafting the CDRs according to Kabat into the IGHV1-2*02 and IGKV4-1*01 human germlines in the IMGT database with VH framework mutations R93S and D97E and VL framework mutation K24R according to IMGT numbering. JIB96 is an IgR comprising two IgG-binding domains per molecule with a heterodimeric Fc, as depicted in FIG. 4I, and was generated using 4 chains, the first light chain comprised Hz17F12-2xVL-kappabDS (SEQ ID NO: 423), the first heavy chain comprised Hz17F12-2xVH-C4CH1bDS-Fc-LALAPA-hole (SEQ ID NO: 424) with P126S and K213E mutations in the CH1 and P126S in the C4CH1spacer to ablate Hz17F12 self-binding, the second light chain comprised SP34-hLC7NoDS (SEQ ID NO: 401) and the second heavy chain comprised SP34-VL-C4CH1NoDS-Fc-LALAPA-knob (SEQ ID NO: 425) with P126S and K213E mutations in the CH1 to ablate Hz17F12 self-binding. JIB97 is similar to JIB95 using the heavy chain Hz17F12-2xVH-C4CH1bDS-αHSA-VHH-his but with TR66 off the LC in Hz17F12-2xVL-kappabDS-TR66-ScFv (SEQ ID NO: 426) as depicted in FIG. 5I (without the half-life extension domain).

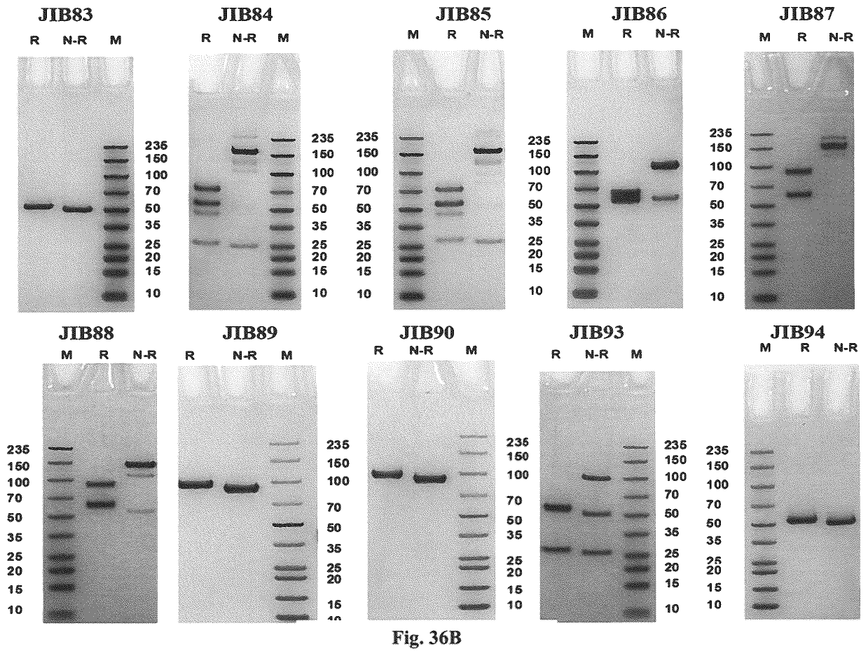

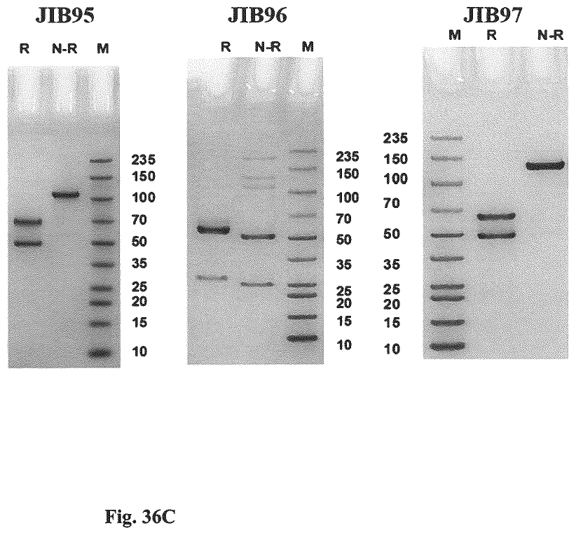

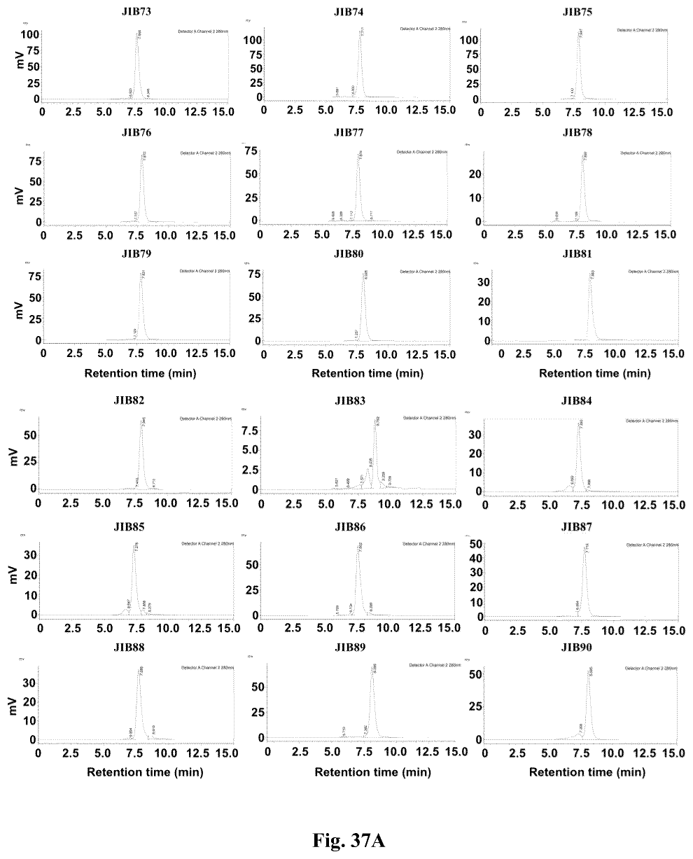

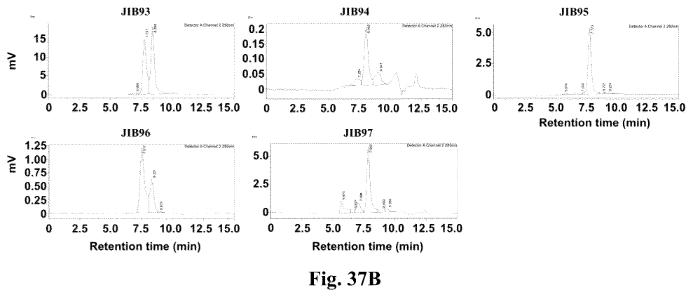

Table 13, FIG. 36A, FIG. 36B, FIG. 36C, FIG. 37A, and FIG. 37B show that all of the IgRs were well expressed with high or moderate purity after single step purification with the exception of JIB91 and JIB92 which did not have detectable protein expression and JIB93 with 44.7% monomer by SEC. All three of these constructs comprised the dual HzHP6017B VH-VH/VL-VL design with constant domain spacers between. In contrast, JIB49 comprised the same dual HP6017 VH-VH/VL-VL design using the same spacers as JIB91 and JIB92 but was capable of expression indicating the humanized sequence has structural differences relative to the wild type mouse sequence contributing to the loss of expression for the dual variable chain format. While JIB93 comprised the dual HzHP6017B VH-VH/VL-VL design, the molecular weights of the chains on the reduced SDS-PAGE gel indicates the HzHP6017B heavy and light chain were not recovered as the molecular weights were more consistent with the SP34 heavy and light chains. The SP34 heavy chain comprising Fc mutations N276K, L309V to ablate HzHP6017B self-binding was recovered indicating these mutations did not interfere with expression and protein recovery by Protein A affinity chromatography. Additionally, the Hz17F12 dual VH-VH/VL-VL constructs, with the same VL-VL spacers and similar VH-VH spacers as JIB91 and JIB92 did not have issues with expression whether with an Fc (JIB96) or without an Fc (JIB95, JIB97) indicating the loss of expression for dual HzHP6017B VH-VH/VL-VL designs are specific to the HzHP6017B clone.

It is notable from SEC and SDS-PAGE that JIB84 and IB85, which comprised four unique polypeptide chains including heterodimer Fc regions, were well expressed and of high purity with 84.2% and 84.6% monomer, respectively. Importantly, JIB84 comprised a first light chain/heavy chain pair using CH1bDS/kappabDS buried interchain disulfide and a second light chain/heavy chain pair comprising CH1NoDS/kappaNoDS without an interchain disulfide; and JIB85 comprised a first light chain/heavy chain pair using CH1bDS/kappabDS and a second light chain/heavy chain pair comprising CH1NoDS/lambdaNoDS without an interchain disulfide. The high purity results indicate the use of a differential interchain disulfide strategy between two light chain/heavy chain pairs in a single molecule enables the ability to produce high purity multimeric proteins where the constant light chain and heavy chains pair with their intended partners reinforcing the findings for JIB47 in Example 21 which used a CH1bDS/kappabDS buried sulfide and CH1-WT/lambda-WT natural disulfide strategy. This also indicates that a differential interchain disulfide strategy enables the use of constant light chain/heavy chain pairs that have the same light chain type (such as CH1/kappa and CH1/kappa or CH1/lambda and CH1/lambda) or different light chain types (such as CH1/kappa and CH1/lambda). The purity data also shows that the three chain, two chain and single chain IgR designs of JIB86; JIB87 and JIB88; and JIB89 and JIB90 are all tractable strategies to enable two immunoglobulin binding domains, one immune cell surface protein binding domain and half life extension within a single IgR molecule.

TABLE 13
Protein Expression and % Monomer by
SEC for IgR produced in Example 23.

| Protein | CHO (mg/L) | SEC % Monomer |
|---|---|---|
| JIB73 | 182 | 94.9% |
| JIB74 | 132 | 92.8% |
| JIB75 | 106 | 97.5% |
| JIB76 | 122 | 97.7% |
| JIB77 | 76 | 97.4% |
| JIB78 | 35 | 99.2% |
| JIB79 | 95 | 96.7% |
| JIB80 | 84 | 96.8% |
| JIB81 | 47 | 100.0% |
| JIB82 | 76 | 97.0% |
| JIB84 | 56 | 84.2% |
| JIB85 | 46 | 84.6% |
| JIB86 | 114 | 94.0% |
| JIB87 | 81 | 89.4% |
| JIB88 | 116 | 91.8% |
| JIB89 | 87 | 93.8% |
| JIB90 | 48 | 84.4% |
| JIB91 | Not Detected | Not Applicable |
| JIB92 | Not Detected | Not Applicable |
| JIB93 | 23 | 44.70% |
| JIB94 | 2 | 66.1% |
| JIB95 | 11 | 84.9% |
| JIB96 | 11 | 69.5% |
| JIB97 | 3 | 62.7% |

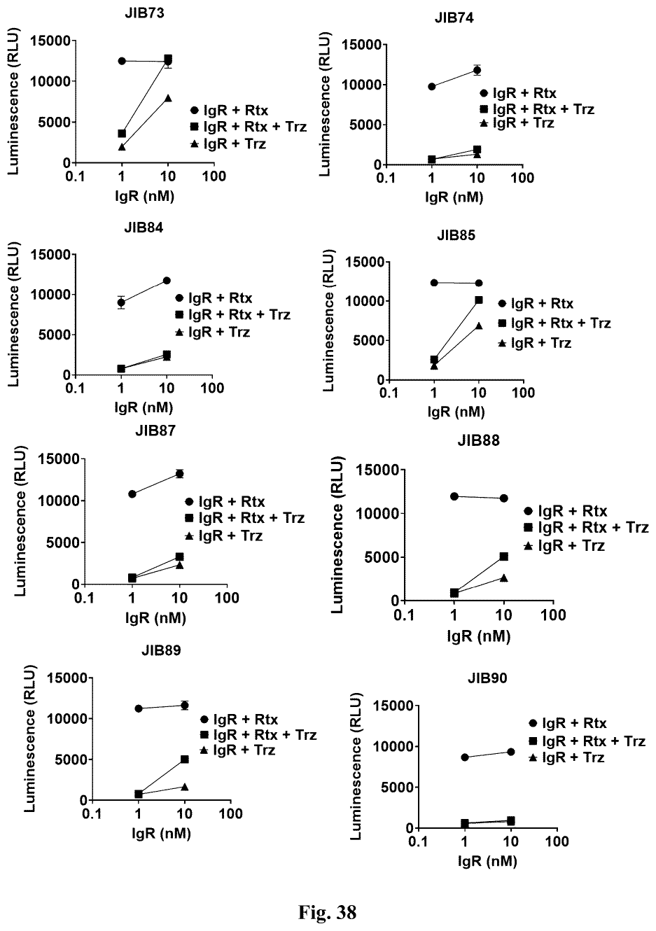

The anti-CD3 IgRs JIB73, JIB74, JIB84, JIB85, JIB87, JIB88, JIB89 and JIB90 were evaluated at 10 nM and 1 nM for their ability to bridge CD20+ Raji B cells with Jurkat T cells and mediate T cell activation, in the presence of 10 nM Rituximab with or without 1 mg/mL Trastuzumab or with 1 mg/mL Trastuzumab without Rituximab using the bioassay method described in Example 8 and Example 20. FIG. 38 compares SP34 versus HzUCHT1 clones in the same four unique IgR constructs. All of the IgRs show potent T cell activation in the presence of 10 nM and 1 nM Rituximab that is substantially greater than in the presence of Trastuzumab. Across all four designs, IgRs using SP34 (JIB73, JIB85, JIB88, JIB89) had a substantially greater activity in the presence of Rituximab and 1 mg/mL Trastuzumab than in the presence of 1 mg/mL Trastuzumab alone, did not have substantially less activity in the presence of Rituximab and 1 mg/mL Trastuzumab than in the presence of Rituximab alone. The relative activities for the four IgRs using HzUCHT1 (JIB74, JIB84, JIB87, JIB90) in the presence of Rituximab and 1 mg/mL Trastuzumab versus with 1 mg/mL Trastuzumab alone or Rituximab alone were less pronounced than with SP34. This data shows that fusing an anti-HSA domain to the Fab-like structure in JIB73 and JIB74 does not interfere with function. The positive data for JIB84 and JIB85 shows using a differential interchain disulfide strategy between two light chain and heavy chain pairs can create functional pairs of pairs maintaining specific target binding for multispecific proteins with high potency, high expression and high purity for either CH1/kappa and CH1/kappa pairs or CH1/kappa and CH1/lambda pairs. It is also noteworthy that the single chain designs (JIB89 and JIB90), with a D1-D2-D3-D4 strategy where D1 and D3 are immunoglobulin binding domains, D2 is an immune cell surface protein binding domain and D4 is a serum albumin binding domain, all show potent activity in the presence of Rituximab alone and the SP34 design has a high ratio of activity between IgR combined with Rituximab and 1 mg/mL Trastuzumab relative to IgR combined with 1 mg/mL Trastuzumab alone, in this case a raw signal RLU ratio of approximately 3.

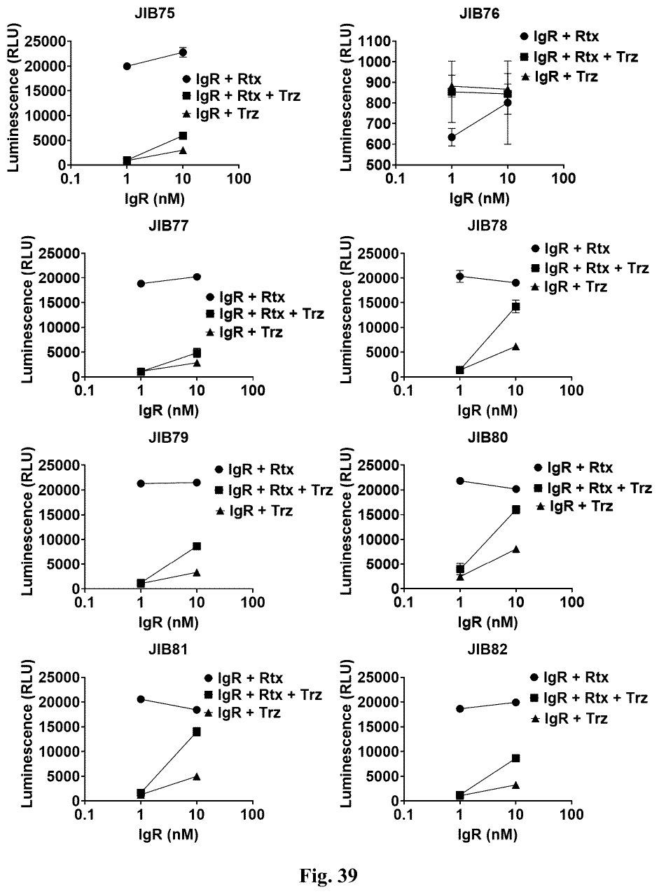

The anti-CD3 IgRs JIB75, JIB76, JIB77, JIB78, JIB79, JIB80, JIB81 and anti-TCR IgR JIB82 all used the Fab-like structure in FIG. 5B with two FcγR2A fused to the C-terminus of the constant light chain and heavy chains. were evaluated at 10 nM and 1 nM for their ability to bridge CD20+ Raji B cells with Jurkat T cells and mediate T cell activation, in the presence of 10 nM Rituximab with or without 1 mg/mL Trastuzumab or with 1 mg/mL Trastuzumab without Rituximab using the bioassay method described in Example 8 and Example 20. FIG. 39 shows that all of the IgRs, with the exception of HzTR66-based JIB76, exhibited a substantially greater activity in the presence of Rituximab or Rituximab and 1 mg/mL Trastuzumab than in the presence of 1 mg/mL Trastuzumab alone. The anti-CD3 IgRs JIB78, JIB79, JIB80, JIB81 and the anti-TCR IgR all exhibited T cell activity in the presence of Rituximab and 1 mg/mL Trastuzumab that was not substantially less than in the presence of Rituximab alone indicating IgRs can function across multiple TCR complex epitopes including different CD3 co-receptors and TCR chains.

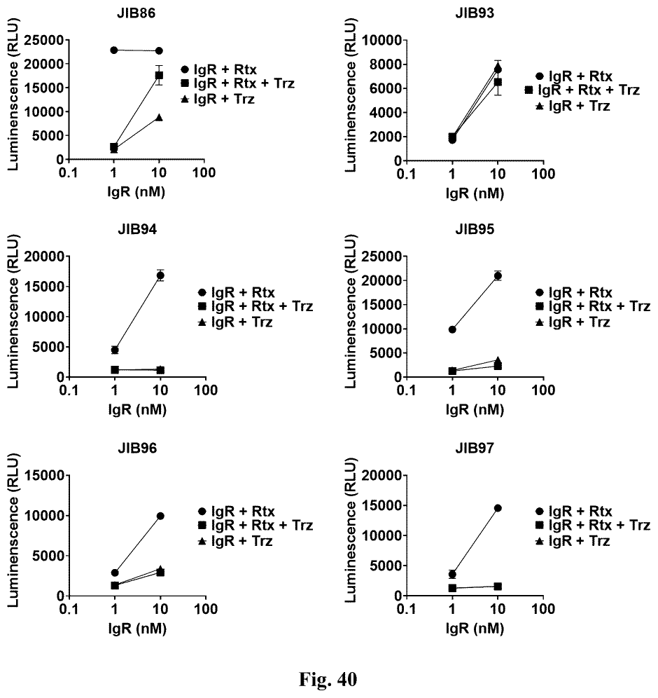

The anti-CD3 IgRs JIB86, JIB93, JIB94, JIB95, JIB96, JIB97 all used the Fab-like structure in FIG. 5B with two FcγR2A fused to the C-terminus of the constant light chain and heavy chains. were evaluated at 10 nM and 1 nM for their ability to bridge CD20+ Raji B cells with Jurkat T cells and mediate T cell activation, in the presence of 10 nM Rituximab with or without 1 mg/mL Trastuzumab or with 1 mg/mL Trastuzumab without Rituximab using the bioassay method described in Example 8 and Example 20. FIG. 40 shows that, with exception of JIB93, all of the IgRs evaluated exhibited substantially greater T cell activation in the presence of Rituximab than in the presence of Trastuzumab alone. The lack of specificity between Rituximab and Trastuzumab for JIB93 is consistent with the purity results where the HP6017B VH-VH/VL-VL strategy appears to eliminate HP6017B heavy and light chain expression while still expressing SP34 light and heavy chain by SDS-PAGE and likely leaving them unpaired to their HP6017B counterparts lending to high nonspecific binding and activation in the T cell activation assay. The T cell activity for JIB86 in the presence of Rituximab and 1 mg/mL Trastuzumab was substantially greater than with 1 mg/mL Trastuzumab alone and not substantially less than with Rituximab alone. While the JIB94 SP34-Fab-like IgR with two HzHP6017B-ScFv (one off each constant chain) and the 17F12-based JIB95, JIB96 and JIB96 did not exhibit a high ratio of T cell activation in the presence of Rituximab and 1 mg/mL Trastuzumab relative to Trastuzumab alone it is important to note that four chain, JIB96, comprising dual Hz17F12 VH-VH/VL-VL using the C4CH1spacer and kappaspacer along with a buried disulfide and the self-binding C4CH1 knockout mutations successfully pair with the SP34 heavy and light chain pair using the C4CH1 knockout mutations and lacking a natural disulfide providing further evidence that a differential interchain disulfide strategy is a tractable means to ensure intended light chain and heavy chain pairing to retain target binding and function.

### Example 24. Expression, Purification and Evaluation of αCD3, αCD28, αCD137 and αCD89 IgRs with Multivalent Binding to IgG Via Multiple Anti-Fc or FcγR Domains

IgG and Fc-binding heterologous polypeptides and heteromultimeric proteins were expressed and purified as described in Example 1 and Example 11.

Anti-CD28 IgRs were generated by VH and VL pairs fused to CH1bDS and kappabDS with human FcγR2A fused to the C-terminus of both the heavy chain (with a his tag) and light chain via (G4S)3 linkers to form Hz2E12-based JIB100 (SEQ ID NO: 427, SEQ ID NO: 428), Hz9.3-based JIB101 (SEQ ID NO: 429, SEQ ID NO: 430), Hz8GA8-based JIB102 (SEQ ID NO: 431, SEQ ID NO: 432), Hz9D7-based JIB103 (SEQ ID NO: 433, SEQ ID NO: 434), HzTN228-based JIB104 (SEQ ID NO: 435, SEQ ID NO: 436), Hz28.3-based JIB105 (SEQ ID NO: 437, SEQ ID NO: 438), Hz5.11A1-based JIB106 (SEQ ID NO: 439, SEQ ID NO: 440), Hz5.11A1-C55S-based JIB107 (SEQ ID NO: 441, SEQ ID NO: 440) and TY24876-based JIB108 (SEQ ID NO: 442, SEQ ID NO: 443) as depicted in FIG. 5B. Hz2E12 VH SEQ ID NO: 444 and VL SEQ ID NO: 445 as well as Hz9.3 VH SEQ ID NO: 446 and VL SEQ ID NO: 447 were designed by grafting the CDRs according to Kabat into the IGHV4-59*01 and IGKV4-1*01 human germlines in the IMGT database with VL framework mutation K24R according to IMGT numbering. Hz8GA8 VH SEQ ID NO: 448 and VL SEQ ID NO: 449 were designed by grafting the CDRs according to Kabat into the IGHV1-2*02 and IGKV1-12*01 human germlines in the IMGT database with VH framework mutations R93S and D97E and VL framework mutation V11L according to IMGT numbering. Hz9D7 VH SEQ ID NO: 450 and VL SEQ ID NO: 451 as well as HzTN228 VH SEQ ID NO: 452 and VL SEQ ID NO: 453 were designed by grafting the CDRs according to Kabat into the IGHV4-59*01 and IGKV1-12*01 human germlines in the IMGT database with VL framework mutation V11L according to IMGT numbering. Hz28.3 VH SEQ ID NO: 454 and VL SEQ ID NO: 455 were designed by grafting the CDRs according to Kabat into the IGHV1-2*02 and IGKV1-12*01 human germlines in the IMGT database with VH framework mutations R93S and D97E and VL framework mutation V11L according to IMGT numbering. 5.11A1 WT VH SEQ ID NO: 456 and VL SEQ ID NO: 457 were humanized to Hz5.11A1 VH SEQ ID NO: 458 and VL SEQ ID NO: 459 by grafting the CDRs according to Kabat into the IGHV1-2*02 and IGKV1-12*01 human germlines in the IMGT database with VH framework mutations R93S and D97E and VL framework mutation V11L according to IMGT numbering. Hz5.11A1-C55S VH SEQ ID NO: 460 was generated by framework mutation C55S in SEQ ID NO: 458 according to IMGT numbering. Additionally, JIB109 (SEQ ID NO: 461) was generated by fusing human FcγR2A to the N-terminus of 1h-79-807-sdAb-VL (SEQ ID NO: 462) followed by a second FcγR2A and his tag via (G4S)3 linkers as depicted in FIG. 2C in a rearranged order.

Anti-CD3 IgR JIB110 was generated by SP34-VH-CH1NoDS-HzFc-10-VHH-his (SEQ ID NO: 463) and SP34-VL-hLC7NoDS-HzFc-10-VHH (SEQ ID NO: 464) as depicted in FIG. 5B. Similarly, JIB111 was generated by SP34-VH-CH1NoDS-anti-Fc-HzVHH2-his (SEQ ID NO: 465) and SP34-VL-hLC7NoDS-anti-Fc-HzVHH2 (SEQ ID NO: 466) as depicted in FIG. 5B. JIB112 was generated by using 20G6 VH (SEQ ID NO: 467) and VL (SEQ ID NO: 468) to make heavy chain 20G6-VH-CH1bDS-FcγR2A-his (SEQ ID NO: 469) and light chain 20G6-VL-kappabDS-FcγR2A (SEQ ID NO: 470) as depicted in FIG. 5B. JIB113 (SEQ ID NO: 471), JIB114 (SEQ ID NO: 472) and JIB115 (SEQ ID NO: 473) were generated by fusing human FcγR2A to the N-terminus of SP34-sdAb-VH (SEQ ID NO: 474), 20G6-sdAb-VH (SEQ ID NO: 475) and Hz06-sdAb-VH (SEQ ID NO: 476), respectively, followed by a second FcγR2A and his tag via (G4S)3 linkers as depicted in FIG. 2C in a rearranged order. HzFc-10-VHH (SEQ ID NO: 477), anti-Fc-HzVHH2 (SEQ ID NO: 478), SP34-sdAb-VH, 20G6-sdAb-VH and Hz06-sdAb-VH and Hz6017-sdAb-VH (SEQ ID NO: 479) were generated by grafting the heavy chain CDRs according to Kabat into the IGHV3-64*04 human germline sequence with framework mutations S24A, V42F, K48Q, Y52A and S54A according to IMGT numbering. JIB126 was generated by SP34-VH-CH1NoDS-HzHP6017-sdAb-VH-his (SEQ ID NO: 480) and SP34-VL-hLC7NoDS-HzHP6017-sdAb-VH (SEQ ID NO: 481) as depicted in FIG. 5B. JIB127 is an IgR comprising two Fc-binding domains per molecule with a heterodimeric Fc and an immune cell binding ScFv fused onto one light chain, as depicted in FIG. 4J, and was generated using 4 chains, the first light chain comprised HzHP6017B-VL-kappabDS (SEQ ID NO: 482), the first heavy chain comprised HzHP6017B-VH-CH1bD-Fc_mut-LALAPA-hole (SEQ ID NO: 483) with additional Fc mutations N276K, L309V to ablate HzHP6017B self Fc-binding, the second light chain comprised HzHP6017B-VL-kappaNoDS-SP34-ScFv (SEQ ID NO: 484) and the second heavy chain comprised HzHP6017B-VH-CH1bD-Fc_mut-LALAPA-knob (SEQ ID NO: 485) with additional Fc mutations N276K, L309V to ablate HzHP6017B self Fc-binding. To address the expression issues with the dual HzHP6017 VH-VH/VL-VL designs in Example 23, three different spacer strategies were evaluated between the VH-VH and VL-VL pairs. JIB128, JIB129 and JIB130 are identical to JIB92 as depicted in FIG. 5I except: for JIB128, the HzHP6017B VL-VL spacer derived from the constant kappa domain was replaced with a shorter spacer derived from the constant kappa light chain (SEQ ID NO: 486) forming HzHP6017-2xVLshort-kappabDS-SP34-ScFv (SEQ ID NO: 487) and co-expressed with JIB92 HC (SEQ ID NO: 411); for JIB129, the HzHP6017B VH-VH spacer derived from the constant CH1 domain was replaced with a shorter spacer derived from the constant CH1 chain (SEQ ID NO: 488) forming HzHP6017B-2xVHshort-CH1bDS-αHSA-VHH-his (SEQ ID NO: 489) and co-expressed with JIB92 LC (SEQ ID NO: 413); for JIB130 the HzHP6017B VH-VH and VL-VL spacers derived from IgG constant domains were replaced by (G4S)₃ spacers forming HzHP6017-2xVLG4S3-kappabDS-SP34-ScFv (SEQ ID NO: 490) and HzHP6017B-2xVHG4S3-CH1bDS-αHSA-VHH-his (SEQ ID NO: 491).

Anti-CD137 IgRs were generating by VH and VL pairs fused to CH1bDS and kappabDS or lambdabDS (for P566) with human FcγR2A fused to the C-terminus of both the heavy chain (with a his tag) and light chain via (G4S)3 linkers to form 5B9-based JIB116 (SEQ ID NO: 492, SEQ ID NO: 493), Hz5B9-based JIB117 (SEQ ID NO: 494, SEQ ID NO: 495), Urelumab-based JIB118 (SEQ ID NO: 496, SEQ ID NO: 497), P566-based JIB119 (SEQ ID NO: 498, SEQ ID NO: 499), Hz4B4-1-based JIB120 (SEQ ID NO: 500, SEQ ID NO: 501), Hz4B4-2-based JIB121 (SEQ ID NO: 502, SEQ ID NO: 503) as depicted in FIG. 5B. Hz4B4-1 is a variant of Hz4B4-2 with seven-fold weaker affinity (Hz4B4-1-VH SEQ ID NO: 504, Hz4B4-1-VL SEQ ID NO: 505). Hz5B9 VH SEQ ID NO: 506 and VL SEQ ID NO: 507 were designed by grafting the CDRs according to Kabat into the IGHV4-59*01 and IGKV2-28*01 human germlines in the IMGT database.

Anti-CD89 IgRs were generated by VH and VL pairs fused to CH1bDS and kappabDS with human FcγR2A fused to the C-terminus of both the heavy chain (with a his tag) and light chain via (G4S)3 linkers to form 14A8-based JIB122 (SEQ ID NO: 508, SEQ ID NO: 509), 8D2-based JIB123 (SEQ ID NO: 510, SEQ ID NO: 511), A77-based JIB124 (SEQ ID NO: 512, SEQ ID NO: 513) and HzA77-based JIB125 (SEQ ID NO: 514, SEQ ID NO: 515) as depicted in FIG. 5B. HzA77 VH (SEQ ID NO: 516) and VL (SEQ ID NO: 517) were designed by grafting the CDRs according to Kabat into the IGHV1-2*02 and IGKV2-28*01 human germlines in the IMGT database with VH framework mutations R93S and D97E according to IMGT numbering.

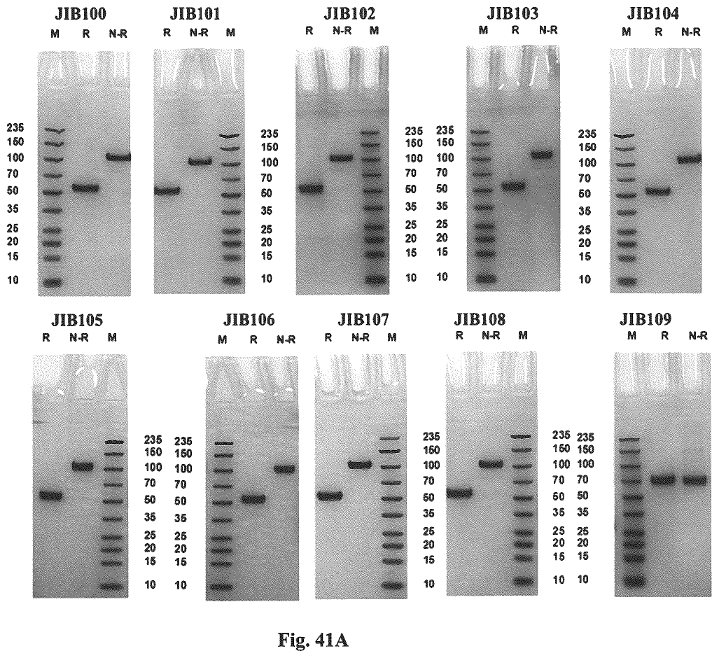

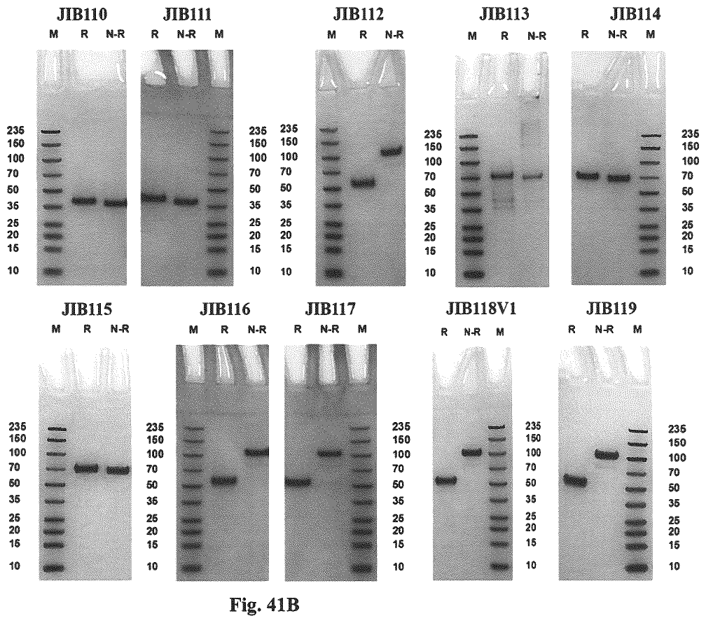

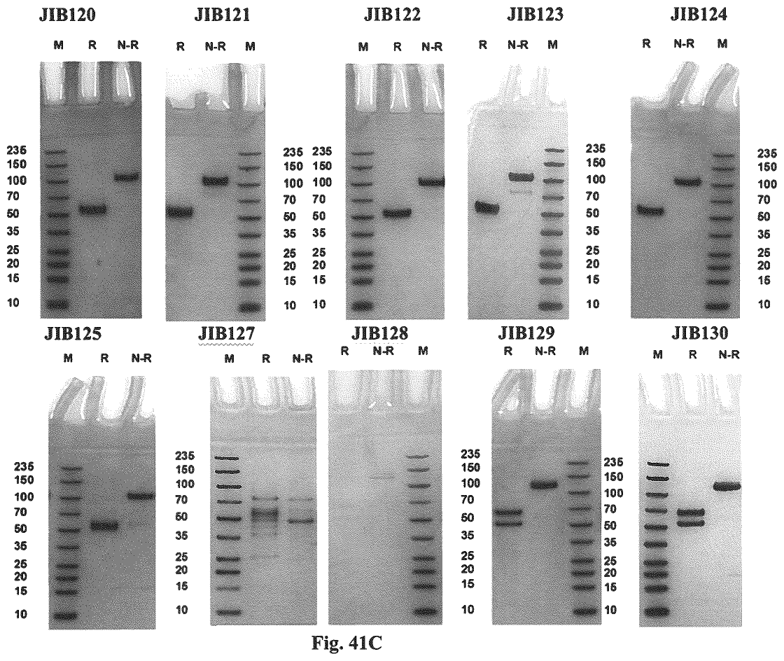

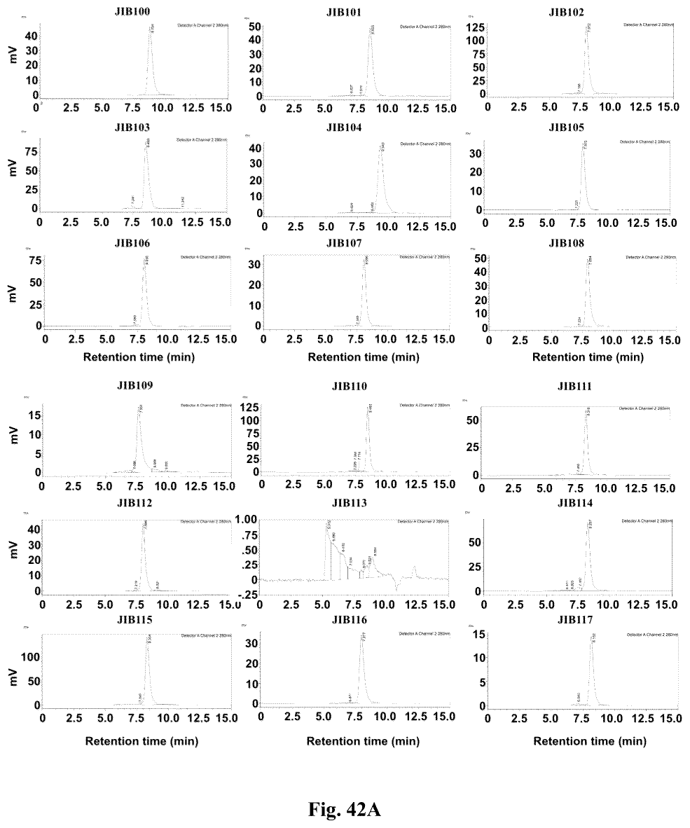

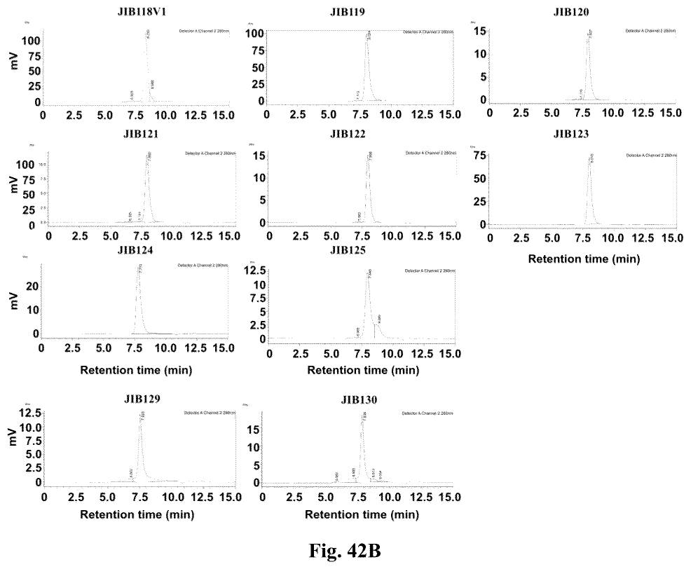

Table 14, FIG. 41A, FIG. 41B, FIG. 41C, FIG. 42A, and FIG. 42B show that all of the IgRs were well expressed with high purity after single step purification with the exception of JIB113, JIB126, JIB127 and JIB128. JIB113 was an attempt at converting the SP34 VH/VL pair antibody into a variable heavy chain only single domain (sdAb-VH). While protein was recovered and relatively pure by SDS-PAGE, the % monomer was quite low by SEC with only 12.3% indicating non-specific interactions with the matrix or itself. Similarly, JIB126, which used the HzHP6017B VH/VL pair converted into a sdAb-VH and fused to each constant chain of an SP34 Fab did not express indicating the conversion into an sdAb-VH was unsuccessful. In contrast, the conversion of the Hz06 and 20G6 VH/VL pairs into sdAb-VH constructs was successful from an expression and purity standpoint. The JIB102 IgR using humanized Hz8GA8 VH/VL sequences was 98.8% monomer which is in stark contrast with the wildtype 8GA8 VH/VL construct JIB70 from Example 22 where the % monomer was 14.7%. This indicates the humanization strategy for Hz8GA8 had the additional benefit of improving its biophysical characteristics.

JIB127 evaluated a Fc-Fc heterodimer with the HzHP6017B VH/VL pair on each Fc arm and an SP34 ScFv off of one of the HzHP6017B light chains using a differential interchain disulfide pairing strategy however no protein was recovered after single purification despite the SDS-PAGE gel of the supernatant (FIG. 41C) showing some protein was expressed in the 25 kDa and 50 kDa region in the reduced lane. These results in congruence with previous results indicate the N276K and L309V mutations may only partially reduce HP6017 or HzHP6017B self-binding to an Fc region. JIB128, JIB129 and JIB130 evaluated alternative spacers between the HzHP6017B dual VH-VH/VL-VL design to address the challenges previously observed with expression for HzHP6017B VH-VH/VL-VL design JIB91, JIB92 and JIB93 which all used CH1spacer/kappaspacer. The alternative spacer strategies included CH1spacer/shortkappaspacer (JIB128), shortCH1spacer/kappaspacer (JIB129) and (G4S)₃/(G4S)₃. The SEC results in Table 14 show that having a short kappa spacer between the variable light chains did not resolve expression issues while having a short CH1 spacer or flexible (G4S)₃ spacers between both variable domain pairs resolved the expression and purity challenges for the dual VH-VH/VL-VL design when using the HzHP6017B clone.

TABLE 14
Protein Expression and % Monomer by
SEC for IgR produced in Example 24.

| Protein | CHO (mg/L) | SEC % Monomer |
|---|---|---|
| JIB100 | 41 | 100.0% |
| JIB101 | 52 | 95.9% |
| JIB102 | 160 | 98.8% |
| JIB103 | 113 | 94.6% |
| JIB104 | 86 | 98.0% |
| JIB105 | 27 | 99.0% |
| JIB106 | 57 | 96.8% |
| JIB107 | 118 | 98.5% |
| JIB108 | 37 | 100.0% |
| JIB109 | 25 | 87.4% |
| JIB110 | 123 | 95.4% |
| JIB111 | 61 | 92.1% |
| JIB112 | 33 | 97.6% |
| JIB113 | 6 | 12.3% |
| JIB114 | 53 | 91.8% |
| JIB115 | 197 | 92.9% |
| JIB116 | 32 | 98.9% |
| JIB117 | 45 | 96.2% |
| JIB118V1 | 59 | 90.3% |
| JIB119 | 133 | 99.8% |
| JIB120 | 40 | 98.7% |
| JIB121 | 29 | 97.7% |
| JIB122 | 39 | 99.6% |
| JIB123 | 111 | 100.0% |
| JIB124 | 55 | 100.0% |
| JIB125 | 37 | 78.5% |
| JIB126 | Not Detected | Not Applicable |
| JIB127 | Low Detection | Not Applicable |
| JIB128 | 0.3 | Not Applicable |
| JIB129 | 43 | 90.0% |
| JIB130 | 13 | 78.5% |

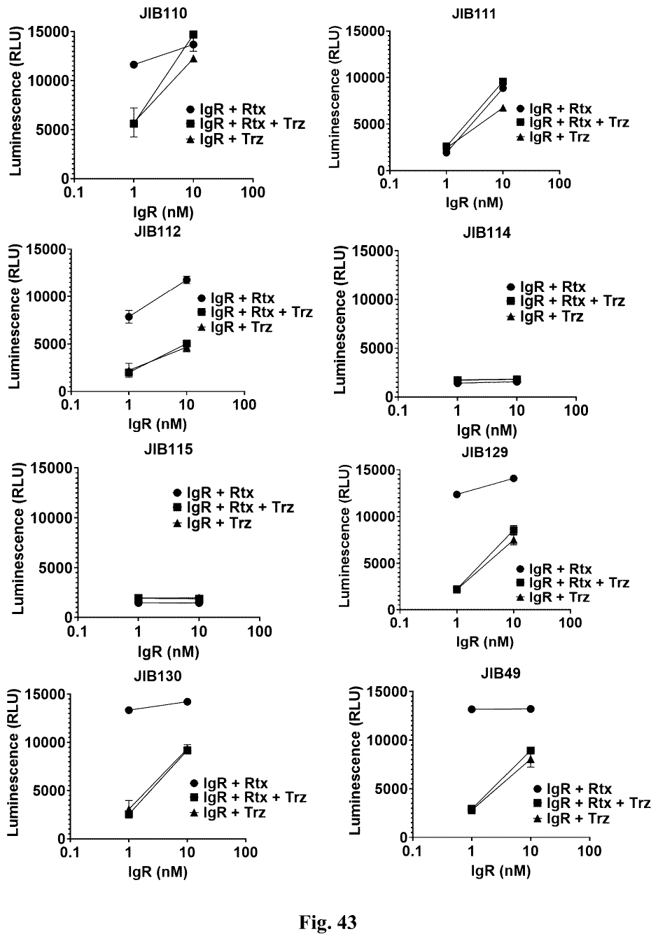

The anti-CD3 IgRs JIB110, JIB111, JIB112, JIB114, JIB115, JIB129, JIB130 and JIB49 were evaluated at 10 nM and 1 nM for their ability to bridge CD20+ Raji B cells with Jurkat T cells and mediate T cell activation, in the presence of 10 nM Rituximab with or without 1 mg/mL Trastuzumab or with 1 mg/mL Trastuzumab without Rituximab using the bioassay method described in Example 8 and Example 20. The results in FIG. 43, show that JIB110, JIB111, JIB112, JIB129, JIB130 and JIB49 show substantially greater T cell activation at one or both concentrations of Rituximab than with 1 mg/mL Trastuzumab. JIB110 and JIB111 both showed T cell activation in the presence of Rituximab at 10 nM and 1 mg/mL Trastuzumab that was not substantially less than 10 nM Rituximab alone. These results indicate the humanization of Fc-10-VHH anti-Fc-HzVHH2 was successful. In contrast, while the JIB112, 20G6-based Fab-like IgR with 2 two FcγR2A off of each LC and HC C-terminus was functional, the 20G6 and Hz06 FcγR2A-sdAb-VH-FcγR2A (JIB114, JIB115, respectively) fusions did not retain their function from the VH/VL pair conversion into single domain VH only constructs. The HzHP6017B dual VH-VH/VL-VL Fab-like IgRs with FcγR2A off of each LC and HC C-terminus were both potent T cell activators in the presence of Rituximab alone and similar to that of the WT HzHP6017 dual VH-VH/VL-VL IgR JIB49 indicating the shortCH1spacer/kappaspacer spacer between the VH-VH/VL-VL pair (JIB129) or the G4S₃ spacer between both the VL-VL and VH-VH pairs (JIB130) not only enabled expression and high purity for the HzHP6017 clone but also retained potent function.

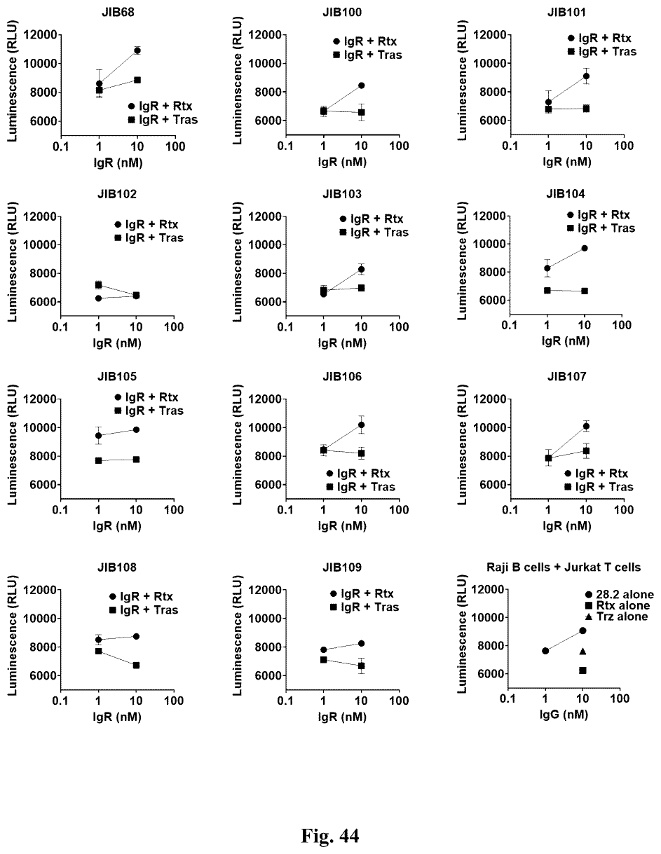

The anti-CD28 IgRs JIB68, JIB100, JIB101, JIB102, JIB03, JIB04, JIB105, JIB106, JIB107, JIB108 and JIB109 were evaluated at 10 nM and 1 nM for their ability to bridge CD20+ Raji B cells with Jurkat T cells and mediate T cell co-stimulation in the presence of 10 nM Rituximab, to evaluate target cell specific T cell co-stimulation activity, or with 10 nM Trastuzumab, to evaluate nonspecific T cell co-stimulation activity, using the method described in Example 22. Rituximab alone at 10 nM, Trastuzumab alone at 10 nM and cells alone were run as negative controls and anti-CD28 mIgG1 clone CD28.2 alone at 10 nM and 1 nM was used as a positive T cell co-stimulation activity control. The results in FIG. 44 show that all of the IgRs evaluated were capable of mediating T cell co-stimulation, in the presence of 10 nM Rituximab at either 10 nM IgR, 1 nM IgR or both, that is greater or substantially greater than the co-stimulation in the presence of 10 nM Trastuzumab. Many of the IgRs evaluated, including JIB100 (Hz2E12), JIB101 (Hz9.3), JIB103 (Hz9D7) and JIB104 (HzTN228), were the humanized versions of the anti-CD28 IgRs evaluated in Example 22 (JIB67, JIB69, JIB71, JI1B72, respectively) indicating these humanized IgRs maintained their co-stimulation function. JIB102 (Hz8GA8) was not evaluated in Example 22 as the WT clone (JIB70) due to poor purity though it exhibited some function as a humanized clone with high purity at the 1 nM IgR condition. JIB105 (Hz28.3), JIB108 (TY24876) and JIB109 (1h-79-807), which were not evaluated in Example 22, were capable of mediating co-stimulation as a humanized or wild type IgRs. JIB109 is a three domain single peptide chain format with FcγR2A fused to the C-terminus and N-terminus of an anti-CD28 single domain VL antibody (sdAb-VL) indicating this is a tractable IgR structure for enabling functional cell-cell synapses and signaling. JIB106 (Hz5.11A1) and JIB107 (Hz5.11A1-C55S) were humanized versions of the WT clone 5.11A1 that TGN1412 was originally developed from and the humanization and the mutation of a cysteine at position 55 to serine (according to IMGT numbering) reduced the amount of nonspecific co-stimulation signal in the presence of 10 nM Trastuzumab relative to the alternatively humanized 5.11A1 clone TGN1412.

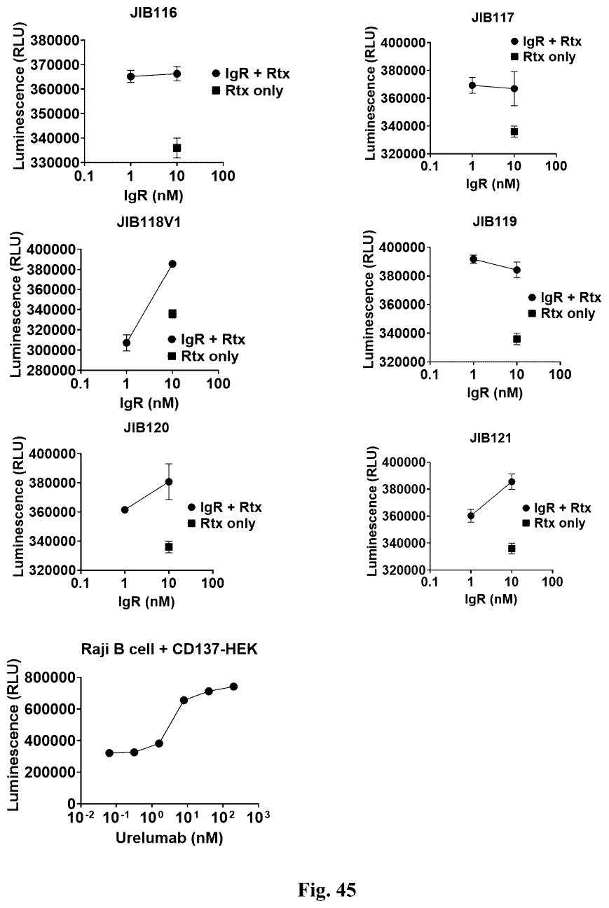

The anti-CD137 IgRs JIB116, JIB117, JIB118V1, JIB119, JIB120 and JIB121 were evaluated at 10 nM and 1 nM for their ability to bridge CD20+ Raji B cells with HEK-Luc-CD137 cells (Biointron), as a surrogate reporter cell for T cells and NK cells, to assess their ability to mediate co-stimulation activity, in the presence of 10 nM Rituximab. Rituximab alone at 10 nM and a titration of Urelumab (Biointron, B315002), a Human IgG4-S228P antibody capable of binding FcγR2B on Raji cells to mediate CD137 crosslinking, alone as a positive control using the bioassay method described in Example 8 and Example 20 but with HEK-Luc-CD137 in place of Jurkat T cells. The results in FIG. 45 show that all of the IgRs, in the presence of 10 nM Rituximab, were capable of mediating co-stimulation activity via targeting and crosslinking CD137 that was substantially greater than in the presence of 10 nM Rituximab alone. These results indicate IgRs are capable of mediating CD137⁺ immune cell co-stimulation function in the presence of antibodies bound to target cells. The positive control of Urelumab titrated at multiple concentrations provided a signal consistent with the assay's ability to detect CD137 crosslinking and co-stimulation activity.

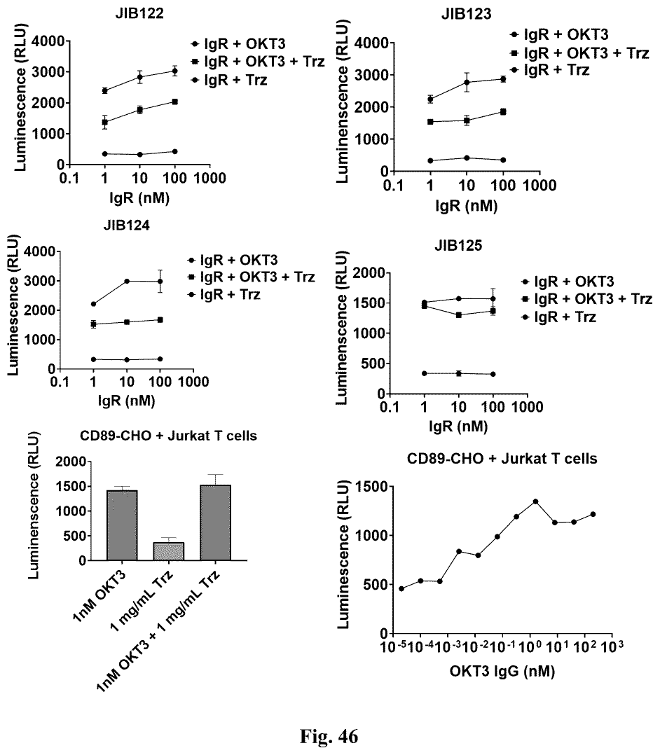

The anti-CD89 IgRs JIB122, JIB123, JIB124 and JIB125 were evaluated for their ability to bridge Jurkat-NFAT-Luc T cells, as a target cell where positive signal indicates crosslinking and is a surrogate reporter for cytotoxicity, and CHO-K1-CD89⁺ cells (Biointron), as a surrogate effector cell representative of CD89⁺ Myeloid cells. IgRs were evaluated at 100 nM, 10 nM and 1 nM for their ability to mediate functional crosslinking activity in the presence of 1 nM OKT3 (Biointron, B6928) with or without 1 mg/mL Trastuzumab, or with 1 mg/mL Trastuzumab without OKT3. Trastuzumab alone at 1 mg/mL and cells alone were run as negative controls and a dose titration of OKT3 (Biointron, B6928) alone or at 1 nM OKT3 with Trastuzumab at 1 mg/mLs were run as positive controls and to set the background signal. The bioassay method described in Example 8 and Example 20 was used but with Jurkat T cells in place of Raji B cells (target cells) and CHO-K1-CD89+ in place of Jurkat T cells (effector cells). The results in FIG. 46 show that all of the IgRs, with exception of JIB125, were capable of mediating CD89 cross-linking and surrogate effector function activity by target cell luciferase production in the presence of 1 nM OKT3 that was substantially greater than in the presence of 1 nM OKT3 alone or 1 mg/mL Trastuzumab alone. Additionally, 14A8 and 8D2-based IgRs were both capable of mediating CD89 cross-linking and surrogate effector function in the presence of 1 nM OKT3 and 1 mg/mL Trastuzumab that was substantially greater than in the presence of 1 mg/mL Trastuzumab alone or 1 mM OKT3 and Trastuzumab alone. These results indicate IgRs are capable of mediating CD89⁺ Myeloid cell effector function against target cells opsonized with antibodies and in the additional presence of high concentrations of nonspecific IgG, similar to conditions in vivo.

### Example 25. Expression, Purification and Evaluation of Mouse Reactive αCD3, αCD28, αCD137 and αCD40 IgRs with Multivalent Binding to IgG Via Multiple Anti-Fc or FcγR Domains

IgG and Fc-binding heterologous polypeptides and heteromultimeric proteins were expressed and purified as described in Example 1 and Example 11. To enable in vivo studies in an immunocompetent syngeneic mouse tumor model known to generate endogenous anti-tumor antibodies, such as CT26 colorectal tumors, 4T1 triple-negative breast tumors, RENCA renal cortical tumors (Zappala et al., 2022), IgRs were generated with domains capable of binding mouse polypeptides on the surface of mouse immune cells and domains capable of binding natural endogenous mouse IgG in vivo including the major subclasses mIgG1, mIgG2a and mIgG2b which make up 90% of the mIgG present in serum/blood in vivo. These IgRs should be capable of bridging endogenous anti-tumor antibodies bound to cancer cells to immune cell receptors to drive cellular activity and efficacy by limiting tumor growth relative to a vehicle control. Additionally, IgRs should synergize when combined with exogenously introduced mAbs against tumor cell targets to enable enhanced anti-tumor efficacy.

IgRs were generating by VH and VL pairs fused to mCH1bDS (SEQ ID NO: 518) and mkappabDS (SEQ ID NO: 519) with mouse mFcγR2b (capable of binding mIgG1, mIgG2a and mIgG2b) or anti-Fc-VHH2 (cross-reacts with mIgG1, mIgG2a and mIgG2b) fused to the C-terminus of both the heavy chain and light chain with αMSA-VHH fused to the C-terminus of the mCH1bDS domain followed by a his tag to form: anti-mCD3 KT3/mFcγR2b-based JIB137 (SEQ ID NO: 520, SEQ ID NO: 521), KT3/anti-Fc-VHH2-based JIB138 (SEQ ID NO: 522, SEQ ID NO: 523) and 2C11/mFcγR2b-based JIB139 (SEQ ID NO: 524, SEQ ID NO: 525); anti-mCD28 TY24876/mFcγR2b-based JIB140 (SEQ ID NO: 526, SEQ ID NO: 527), anti-mCD137 Lob12.3/mFcγR2b-based JIB142 (SEQ ID NO: 528, SEQ ID NO: 529), Lob12.3/anti-Fc-VHH2-based JIB143 (SEQ ID NO: 530, SEQ ID NO: 531), 3H3/mFcγR2b-based JIB144 (SEQ ID NO: 532, SEQ ID NO: 533); anti-mCD40 1C10/mFcγR2b-based JIB145 (SEQ ID NO: 534, SEQ ID NO: 535), 1C10/anti-Fc-VHH2-based JIB146 (SEQ ID NO: 536, SEQ ID NO: 537) and FGK45/mFcγR2b-based JIB147 (SEQ ID NO: 538, SEQ ID NO: 539) as depicted in FIG. 5J. JIB141 was generated by fusing mFcγR2b to the N-terminus of 1h-79-807-sdAb-VL followed by mFcγR2b, αMSA-VHH and a his tag via (G4S)3 linkers forming mFcγR2b-SP1h-79-807-sdAb-VL-mFcγR2b-αMSA-VHH-his (SEQ ID NO: 540) as depicted in FIG. 3B (in a rearranged order).

To have exogenously introduced IgG to combine with IgRs in vivo to treat solid tumors, an anti-EphA2 monoclonal antibody capable of binding mouse tumors such as CT26 was produced by fusing anti-EphA2 VH (SEQ ID NO: 549) and VL (SEQ ID NO: 550) to mouse mIgG1 heavy chain (SEQ ID NO: 551) and mouse lambdal light chain (SEQ ID NO: 552) generating JIB148. Similarly, to have exogenously introduced IgG to combine with IgRs in vivo to treat liquid tumors, an anti-CD19 monoclonal antibody, capable of binding mouse blood tumors such as A20 B cell lymphoma tumors and with preferential binding towards FcγRIV and human FcγR3A/B, was produced by fusing 1D3 VH (SEQ ID NO: 553) and VL (SEQ ID NO: 554) to mouse mIgG2a heavy chain (SEQ ID NO: 555) and mouse kappa light chain (SEQ ID NO: 556) and cultured in the presence of 20 M kifunensine to produce non-fucosylated Fc glycoforms, that have preferential binding towards FcγRIV and human FcγR3A/B, generating anti-mCD19 mAb 1D3-IgG2a-kif.

TABLE 15
Protein Expression and % Monomer by SEC for
mouse reactive IgR produced in Example 25.

| Protein | CHO (mg/L) | SEC % Monomer |
|---|---|---|
| JIB137 | 116 | 82.85% |
| JIB138 | 275 | 96.14% |
| JIB139 | 133 | 82.8% |
| JIB140 | 3 | 83.4% |
| JIB141 | 82 | 4.3% |
| JIB142 | 220 | 91.6% |
| JIB143 | 321 | 93.2% |
| JIB144 | 48 | 82.6% |
| JIB145 | 252 | 91.6% |
| JIB146 | 337 | 93.7% |
| JIB147 | 87 | 70.9% |
| JIB148 | 378 | 89.3% |
| 1D3-IgG2a-kif | 47 | 98.6% |

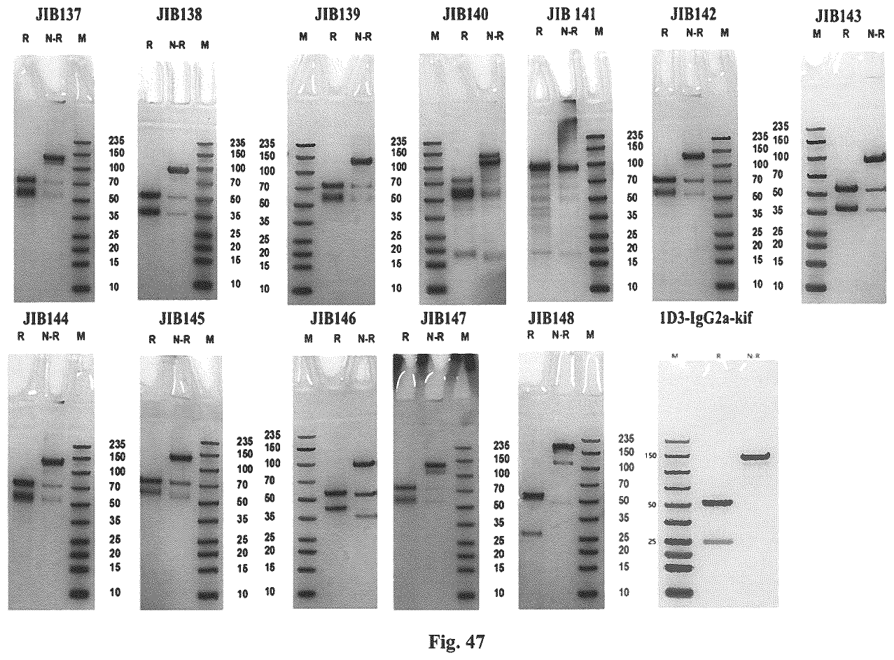

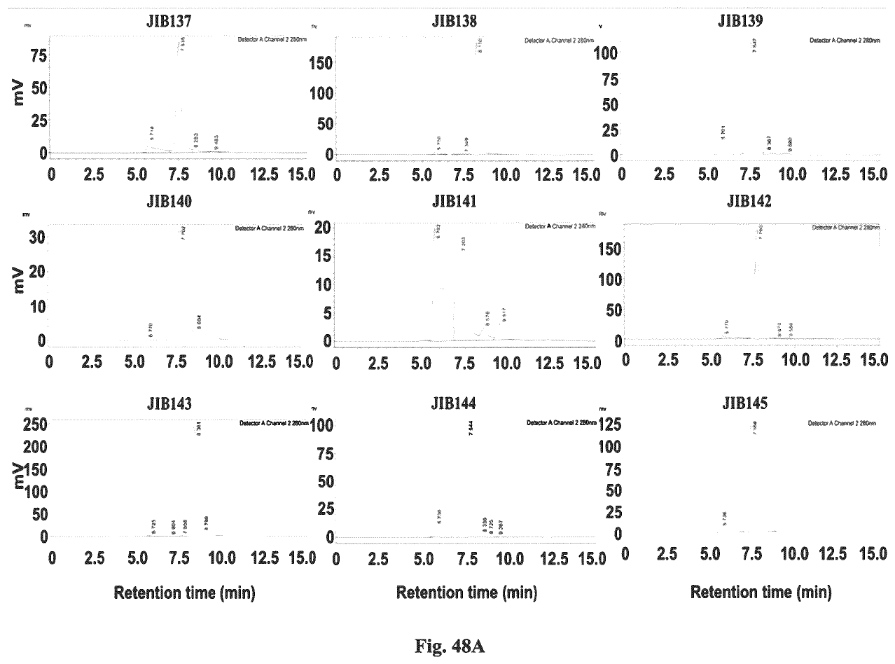

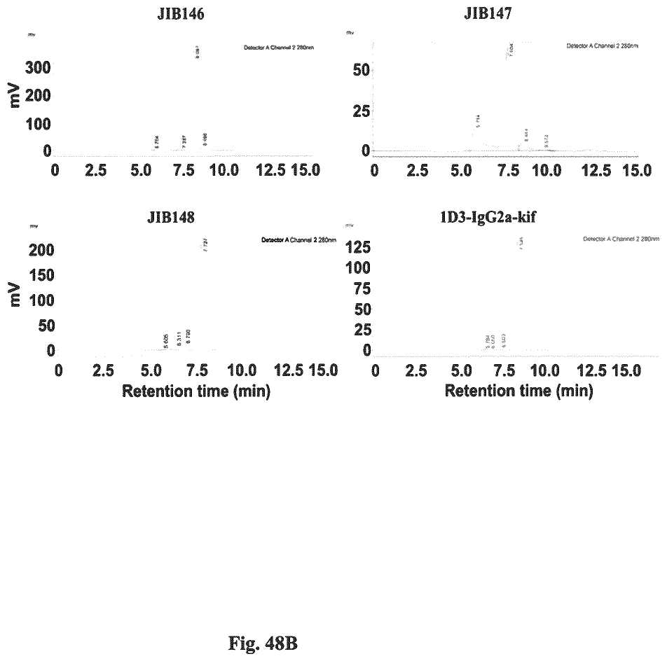

Table 15, FIG. 47, FIG. 48A, and FIG. 48B show that all of the IgRs and IgG were well expressed with high purity after single step purification with exception of JIB141 with a mFcγR2b-SP1h-79-807-sdAb-VL-mFcγR2b-αMSA-VHH-his design having 4.3% monomer by SEC. This is in contrast to the similar JIB109 design, which uses the same anti-CD28 clone, with FcγRA-SP1h-79-807-sdAb-VL-FcγR2A-his having 87.4% monomer after single step purification by SEC.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### REFERENCES

Anthony, R. M., Wermeling, F., Karlsson, M. C. I., & Ravetch, J. V. (2008). Identification of a receptor required for the anti-inflammatory activity of IVIG. Proceedings of the National Academy of Sciences of the United States of America, 105(50), 19571-19578.
Arnold, J. N., Dwek, R. A., Rudd, P. M., & Sim, R. B. (2006). Mannan binding lectin and its interaction with immunoglobulins in health and in disease. Immunology Letters, 106(2), 103-110.
Beck, A., Goetsch, L., Dumontet, C., & Corvaia, N. (2017). Strategies and challenges for the next generation of antibody-drug conjugates. Nature Reviews. Drug Discovery, 16(5), 315-337.
Belin, P., Dassa, J., Drevet, P., Lajeunesse, E., Savatier, A., Boulain, J.-C., & Ménez, A. (2004). Toxicity-based selection of Escherichia coli mutants for functional recombinant protein production: application to an antibody fragment. Protein Engineering, Design & Selection: PEDS, 17(5), 491-500.
Boesch, A. W., Alter, G., & Ackerman, M. E. (2015). Prospects for engineering HIV-specific antibodies for enhanced effector function and half-life. Current Opinion in HIV and AIDS, 10(3), 160-169.
Boesch, A. W., Brown, E. P., Cheng, H. D., Ofori, M. O., Normandin, E., Nigrovic, P. A., Alter, G., & Ackerman, M. E. (2014). Highly parallel characterization of IgG Fc binding interactions. mAbs, 6(4), 915-927.
Bonvin, P., Venet, S., Kosco-Vilbois, M., & Fischer, N. (2015). Purpose-Oriented Antibody Libraries Incorporating Tailored CDR3 Sequences. Antibodies, 4(2), 103-122.
Brinkmann, U., & Kontermann, R. E. (2017). The making of bispecific antibodies. mAbs, 9(2), 182. Brinkmann, U., Reiter, Y., Jung, S. H., Lee, B., & Pastan, I. (1993). A recombinant immunotoxin containing a disulfide-stabilized Fv fragment. Proceedings of the National Academy of Sciences of the United States of America, 90(16), 7538-7542.
Carrier, A., Ducancel, F., Settiawan, N. B., Cattolico, L., Maillbre, B., Ldonetti, M., Drevet, P., Ménez, A., & Boulain, J. C. (1995). Recombinant antibody-alkaline phosphatase conjugates for diagnosis of human IgGs: application to anti-HBsAg detection. Journal of Immunological Methods, 181(2). https://doi.org/10.1016/0022-1759(94)00344-v
Chang, T.-W., Hung, A. F.-H., & Lu, D. C.-S. (2017). Anti-human mIgA antibodies capable of lysing mIgA-B lymphocytes and decreasing IgA production (U.S. Pat. No. 9,688,776). In US patent (U.S. Pat. No. 9,688,776). https://patentimages.storage.googleapis.com/b9/7c/fO/df399842c072a7/US9688776.pdf
Chen, K., & Cerutti, A. (2011). The function and regulation of immunoglobulin D. Current Opinion in Immunology, 23(3), 345-352.
Chothia, C., Novotnf, J., Bruccoleri, R., & Karplus, M. (1985). Domain association in immunoglobulin molecules. The packing of variable domains. Journal of Molecular Biology, 186(3). https://doi.org/10.1016/0022-2836(85)90137-8
Corrales-Aguilar, E., Hoffmann, K., & Hengel, H. (2014). CMV-encoded Fcγ receptors: modulators at the interface of innate and adaptive immunity. Seminars in Immunopathology, 36(6), 627-640.
Dekkers, G., Bentlage, A. E. H., Stegmann, T. C., Howie, H. L., Lissenberg-Thunnissen, S., Zimring, J., Rispens, T., & Vidarsson, G. (2017). Affinity of human IgG subclasses to mouse Fc gamma receptors. mAbs, 9(5), 767-773.
Delespesse, G., Hofstetter, H., & Sarfati, M. (1989). Low-affinity receptor for IgE (FcERII, CD23) and its soluble fragments. International Archives of Allergy and Applied Immunology, 90 Suppl 1, 41-44.
de Vries, E., Koene, H. R., Vossen, J. M., Gratama, J. W., von dem Borne, A. E., Waaijer, J. L., Haraldsson, A., de Haas, M., & van Tol, M. J. (1996). Identification of an unusual Fc gamma receptor IIIa (CD16) on natural killer cells in a patient with recurrent infections. Blood, 88(8), 3022-3027.
Dong, X., Storkus, W. J., & Salter, R. D. (1999). Binding and uptake of agalactosyl IgG by mannose receptor on macrophages and dendritic cells. Journal of Immunology, 163(10), 5427-5434.
Dubin, G., Frank, I., & Friedman, H. M. (1990). Herpes simplex virus type 1 encodes two Fc receptors which have different binding characteristics for monomeric immunoglobulin G (IgG) and IgG complexes. Journal of Virology, 64(6), 2725-2731.
Foote, J., & Winter, G. (1992). Antibody framework residues affecting the conformation of the hypervariable loops. Journal of Molecular Biology, 224(2). https://doi.org/10.1016/0022-2836(92)91010-m
Frey, S., Opazo, F., & Götzke, H. (2018). Target detection using a monovalent antibody (WIPO Patent No. 2018167141:A2). In World Patent (No. 2018167141:A2). https://patentimages.storage.googleapis.com/95/b5/e5/8702c689c5d90e/WO2018167141A2.pdf
Geddie, M. L., Kirpotin, D. B., Kohli, N., Komaga, T., Boll, B., Razlog, M., Drummond, D. C., & Lugovskoy, A. A. (2022). Development of disulfide-stabilized Fabs for targeting of antibody-directed nanotherapeutics. mAbs, 14(1). https://doi.org/10.1080/19420862.2022.2083466 Hagihara, Y., & Saerens, D. (2014). Engineering disulfide bonds within an antibody. Biochimica et Biophysica Acta, 1844(11), 2016-2023.
Halaby, D. M., Poupon, A., & Mornon, J. (1999). The immunoglobulin fold family: sequence analysis and 3D structure comparisons. Protein Engineering, 12(7). https://doi.org/10.1093/protein/12.7.563
Hallam, T. J., Wold, E., Wahl, A., & Smider, V. V. (2015). Antibody conjugates with unnatural amino acids. Molecular Pharmaceutics, 12(6), 1848-1862.
Hamilton, R. G., & Morrison, S. L. (1993). Epitope mapping of human immunoglobulin-specific murine monoclonal antibodies with domain-switched, deleted and point-mutated chimeric antibodies. Journal of Immunological Methods, 158(1), 107-122.
Hermans, W. J. J., Bezemer, S., & Mijnsbergen, Y. M. (2015). Single-domain antigen-binding proteins that bind mammalian IgG (U.S. Pat. No. 9,040,666). In US patent (U.S. Pat. No. 9,040,666). https://patentimages.storage.googleapis.com/a6/30/24/5c6331255c5ef5/US9040666.pdf
Hermans, W. J. J., Blokland, S., van Kesteren, M. A., Horrevoets, J. M., & Detmers, F. J. M. (2017). Antigen-binding protein directed against epitope in the CH1 domain of human IgG antibodies (U.S. Pat. No. 9,850,315). In US patent (U.S. Pat. No. 9,850,315). https://patentimages.storage.googleapis.com/61/e4/6a/1f1b6a48d95d10/US9850315.pdf
Herrmann, M., Krupka, C., Deiser, K., Brauchle, B., Marcinek, A., Ogrinc Wagner, A., Rataj, F., Mocikat, R., Metzeler, K. H., Spiekermann, K., Kobold, S., Fenn, N. C., Hopfner, K.-P., & Subklewe, M. (2018). Bifunctional PD-1×αCD3×αCD33 fusion protein reverses adaptive immune escape in acute myeloid leukemia. Blood, 132(23), 2484-2494.
Hogarth, P. M., & Wines, B. D. (2014). Multimeric Fc receptor polypeptides (U.S. Pat. No. 8,729,247). In US patent (U.S. Pat. No. 8,729,247). https://patentimages.storage.googleapis.com/2a/a6/81/a6474991b98b27/US8729247.pdf
Hulett, & Hogarth, P. M. (1998). The second and third extracellular domains of FcgammaRI (CD64) confer the unique high affinity binding of IgG2a. Molecular Immunology, 35(14-15). https://doi.org/10.1016/s0161-5890(98)00069-8
Jeffrey, S. C., Burke, P. J., Lyon, R. P., Meyer, D. W., Sussman, D., Anderson, M., Hunter, J. H., Leiske, C. I., Miyamoto, J. B., Nicholas, N. D., Okeley, N. M., Sanderson, R. J., Stone, I. J., Zeng, W., Gregson, S. J., Masterson, L., Tiberghien, A. C., Howard, P. W., Thurston, D. E., . . . Senter, P. D. (2013). A potent anti-CD70 antibody-drug conjugate combining a dimeric pyrrolobenzodiazepine drug with site-specific conjugation technology. Bioconjugate Chemistry, 24(7), 1256-1263.
Johnson, L. S., Li, H., & Tuaillon, N. (2010). FcgammaRIIB Fusion Proteins and Compositions Thereof (USPTO Patent No. 20100196372:A1). In US patent (No. 20100196372:A1). https://patentimages.storage.googleapis.com/3c/b3/a3/3703e4a4dd5edb/US20100196372A1.pdf
Junutula, J. R., Raab, H., Clark, S., Bhakta, S., Leipold, D. D., Weir, S., Chen, Y., Simpson, M., Tsai, S. P., Dennis, M. S., Lu, Y., Meng, Y. G., Ng, C., Yang, J., Lee, C. C., Duenas, E., Gorrell, J., Katta, V., Kim, A., . . . Mallet, W. (2008). Site-specific conjugation of a cytotoxic drug to an antibody improves the therapeutic index. Nature Biotechnology, 26(8), 925-932.
Kabat, E. A. (1992). Sequences of Proteins of Immunological Interest. DIANE Publishing.
Karsten, C. M., Pandey, M. K., Figge, J., Kilchenstein, R., Taylor, P. R., Rosas, M., McDonald, J. U., Orr, S. J., Berger, M., Petzold, D., Blanchard, V., Winkler, A., Hess, C., Reid, D. M., Majoul, I. V., Strait, R. T., Harris, N. L., Kohl, G., Wex, E., . . . Kohl, J. (2012). Anti-inflammatory activity of IgG1 mediated by Fc galactosylation and association of FcγRIIB and dectin-1. Nature Medicine, 18(9), 1401-1406.
Keeble, A. H., Khan, Z., Forster, A., & James, L. C. (2008). TRIM21 is an IgG receptor that is structurally, thermodynamically, and kinetically conserved. Proceedings of the National Academy of Sciences of the United States of America, 105(16), 6045-6050.
Leonard, E. K., Pellitero, M. A., Juelg, B., Spangler, J. B., & Arroyo-Curris, N. (2022). Antibody-Invertase Fusion Protein Enables Quantitative Detection of SARS-CoV-2 Antibodies Using Widely Available Glucometers. Journal of the American Chemical Society. https://doi.org/10.1021/jacs.2c02537
Litwin, V., Jackson, W., & Grose, C. (1992). Receptor properties of two varicella-zoster virus glycoproteins, gpI and gpIV, homologous to herpes simplex virus gE and gI. Journal of Virology, 66(6), 3643-3651.
Liu, H., Saxena, A., Sidhu, S. S., & Wu, D. (2017). Fc Engineering for Developing Therapeutic Bispecific Antibodies and Novel Scaffolds. Frontiers in Immunology, 8. https://doi.org/10.3389/fimmu.2017.00038
Liu, L., Lam, C.-Y. K., Alderson, R., Long, V., Yang, Y., Burns, R., Widjaja, L., Li, J., Wolf, C., Ciccarone, V., Tamura, J., Diedrich, G., Bonvini, E., Johnson, S., & Moore, P. A. (2019 Jul. 1). Abstract 1560: Selection of a bispecific trivalent HER2×CD137 TRIDENT format providing optimal tumor-anchored immune co-stimulation. Immunology. Proceedings: AACR Annual Meeting 2019; Mar. 29-Apr. 3, 2019; Atlanta, GA. https://doi.org/10.1158/1538-7445.sabcs18-1560
Miller, M. G., Ing, J. Y., Cheng, M. K.-W., Flitter, B. A., & Moe, G. R. (2013). Identification of a phage-encoded Ig-binding protein from invasive Neisseria meningitidis. Journal of Immunology, 191(6), 3287-3296.
Nakamura, H., Oda-Ueda, N., Ueda, T., & Ohkuri, T. (2018). A novel engineered interchain disulfide bond in the constant region enhances the thermostability of adalimumab Fab. Biochemical and Biophysical Research Communications, 495(1), 7-11.
Namboodiri, A. M., Budkowska, A., Nietert, P. J., & Pandey, J. P. (2007). Fc gamma receptor-like hepatitis C virus core protein binds differentially to IgG of discordant Fc (GM) genotypes. Molecular Immunology, 44(15), 3805-3808.
Oganesyan, V., Mazor, Y., Yang, C., Cook, K. E., Woods, R. M., Ferguson, A., Bowen, M. A., Martin, T., Zhu, J., Wu, H., & Dall'Acqua, W. F. (2015). Structural insights into the interaction of human IgG1 with FcγRI: no direct role of glycans in binding. Acta Crystallographica. Section D, Biological Crystallography, 71 (Pt 11), 2354.
Para, M. F., Baucke, R. B., & Spear, P. G. (1980). Immunoglobulin G(Fc)-binding receptors on virions of herpes simplex virus type 1 and transfer of these receptors to the cell surface by infection. Journal of Virology, 34(2), 512-520.
Patel, K. R., Roberts, J. T., & Barb, A. W. (2019). Multiple Variables at the Leukocyte Cell Surface Impact Fc γ Receptor-Dependent Mechanisms. Frontiers in Immunology, 10, 223.
Pillow, T. H., Tien, J., Parsons-Reponte, K. L., Bhakta, S., Li, H., Staben, L. R., Li, G., Chuh, J., Fourie-O'Donohue, A., Darwish, M., Yip, V., Liu, L., Leipold, D. D., Su, D., Wu, E., Spencer, S. D., Shen, B.-Q., Xu, K., Kozak, K. R., . . . Junutula, J. R. (2014). Site-specific trastuzumab maytansinoid antibody-drug conjugates with improved therapeutic activity through linker and antibody engineering. Journal of Medicinal Chemistry, 57(19), 7890-7899.
Prihoda, D., Maamary, J., Waight, A., Juan, V., Fayadat-Dilman, L., Svozil, D., & Bitton, D. A. (2022). BioPhi: A platform for antibody design, humanization, and humanness evaluation based on natural antibody repertoires and deep learning. mAbs, 14(1), 2020203.
Promsote, W., Xu, L., Hataye, J., Fabozzi, G., March, K., Almasri, C. G., DeMouth, M. E., Lovelace, S. E., Talana, C. A., Doria-Rose, N. A., McKee, K., Hait, S. H., Casazza, J. P., Ambrozak, D., Beninga, J., Rao, E., Furtmann, N., Birkenfeld, J., McCarthy, E., . . . Koup, R. A. (2023). Trispecific antibody targeting HIV-1 and T cells activates and eliminates latently-infected cells in HIV/SHIV infections. Nature Communications, 14(1), 1-13.
Raghavan, M., Chen, M. Y., Gastinel, L. N., & Bjorkman, P. J. (1994). Investigation of the interaction between the class I MHC-related Fc receptor and its immunoglobulin G ligand. Immunity, 1(4), 303-315.
Rueger, P., Schlothauer, T., & Seeber, S. (2018). METHOD FOR PRODUCING SOLUBLE FcR AS Fc-FUSION WITH INERT IMMUNOGLOBULIN Fc-REGION AND USES THEREOF (USPTO Patent No. 20180265564:A1). In US patent (No. 20180265564:A1). https://patentimages.storage.googleapis.com/64/a7/41/defc1adfda2cd5/US20180265564A1.pdf
Saunders, K. O. (2019). Conceptual Approaches to Modulating Antibody Effector Functions and Circulation Half-Life. Frontiers in Immunology, 10, 449325.
Shibata-Koyama, M., Iida, S., Okazaki, A., Mori, K., Kitajima-Miyama, K., Saitou, S., Kakita, S., Kanda, Y., Shitara, K., Kato, K., & Satoh, M. (2009). The N-linked oligosaccharide at FcγRIIIa Asn-45: an inhibitory element for high FcγRIIIa binding affinity to IgG glycoforms lacking core fucosylation. Glycobiology, 19(2), 126.
Shin, J.-S., & Greer, A. M. (2015). The role of FcεRI expressed in dendritic cells and monocytes. Cellular and Molecular Life Sciences: CMLS, 72(12), 2349-2360.
Strohl, W. R. (2015). Fusion Proteins for Half-Life Extension of Biologics as a Strategy to Make Biobetters. BioDrugs: Clinical Immunotherapeutics, Biopharmaceuticals and Gene Therapy, 29(4), 215.
Studnicka, G. M., Soares, S., Better, M., Williams, R. E., Nadell, R., & Horwitz, A. H. (1994). Human-engineered monoclonal antibodies retain full specific binding activity by preserving non-CDR complementarity-modulating residues. Protein Engineering, 7(6). https://doi.org/10.1093/protein/7.6.805
The second and third extracellular domains of FcγRI (CD64) confer the unique high affinity binding of IgG2a. (1998). Molecular Immunology, 35(14-15), 989-996.
Tumey, L. N., Li, F., Rago, B., Han, X., Loganzo, F., Musto, S., Graziani, E. I., Puthenveetil, S., Casavant, J., Marquette, K., Clark, T., Bikker, J., Bennett, E. M., Barletta, F., Piche-Nicholas, N., Tam, A., O'Donnell, C. J., Gerber, H. P., & Tchistiakova, L. (2017). Site Selection: a Case Study in the Identification of Optimal Cysteine Engineered Antibody Drug Conjugates. The AAPS Journal, 19(4), 1123-1135.
UniProt. (n.d.-a). Retrieved Jun. 27, 2024, from https://www.uniprot.org/uniprotkb/P08637/entry
UniProt. (n.d.-b). Retrieved Jun. 27, 2024, from https://www.uniprot.org/uniprotkb/O75015/entry
UniProt. (n.d.-c). Retrieved Jun. 27, 2024, from https://www.uniprot.org/uniprotkb/A0A0B4J1G0/entry
UniProt. (n.d.-d). Retrieved Jun. 27, 2024, from https://www.uniprot.org/uniprotkb/P12318/entry
UniProt. (n.d.-e). Retrieved Jun. 27, 2024, from https://www.uniprot.org/uniprotkb/P31994/publications
UniProt. (n.d.-f). Retrieved Jun. 27, 2024, from https://www.uniprot.org/uniprotkb/P12314/entry
UniProt. (n.d.-g). Retrieved Jun. 27, 2024, from https://www.uniprot.org/uniprotkb/P01833/entry
UniProt. (n.d.-h). Retrieved Jun. 27, 2024, from https://www.uniprot.org/uniprotkb/P24071/entry
UniProt. (n.d.-i). Retrieved Jun. 27, 2024, from https://www.uniprot.org/uniprotkb/Q8WWV6/entry
UniProt. (n.d.-j). Retrieved Jun. 27, 2024, from https://www.uniprot.org/uniprotkb/O60667/entry
UniProt. (n.d.-k). Retrieved Jun. 27, 2024, from https://www.uniprot.org/uniprotkb/P12319/entry
UniProt. (n.-d.-l). Retrieved Jun. 27, 2024, from https://www.uniprot.org/uniprotkb/P06734/entry
UniProt. (n.d.-m). Retrieved Jun. 27, 2024, from https://www.uniprot.org/uniprotkb/P55899/entry
Univ, K. (2010). IgG BINDING PEPTIDE (USPTO Patent No. 20100297606A1). In US patent (No. 20100297606A1). https://eureka.patsnap.com/patent-US20100297606A1
Vaks, L., Litvak-Greenfeld, D., Dror, S., Shefet-Carasso, L., Matatov, G., Nahary, L., Shapira, S., Hakim, R., Alroy, I., & Benhar, I. (2018). Design Principles for Bispecific IgGs, Opportunities and Pitfalls of Artificial Disulfide Bonds. Antibodies (Basel, Switzerland), 7(3). https://doi.org/10.3390/antib7030027
Wang, C., Wu, Y., Wang, L., Hong, B., Jin, Y., Hu, D., Chen, G., Kong, Y., Huang, A., Hua, G., & Ying, T. (2017). Engineered Soluble Monomeric IgG1 Fc with Significantly Decreased Non-Specific Binding. Frontiers in Immunology, 8, 302778.
Website. (n.d.-a). https://www.ncbi.nlm.nih.gov/pmc/articles/PMC3220521/,
Website. (n.d.-b). https://www.uniprot.org/uniprotkb/Q96RD9/entry
Wilson, T. J., Fuchs, A., & Colonna, M. (2012). Cutting edge: human FcRL4 and FcRL5 are receptors for IgA and IgG. Journal of Immunology, 188(10), 4741-4745.
Zappala, F., Higbee-Dempsey, E., Jang, B., Miller, J., Yan, L., Minutolo, N. G., Rosado Gonzilez, G. T., Tsourkas, A., & Ozdemir, B. A. (2022). Rapid, site-specific labeling of “off-the-shelf” and native serum autoantibodies with T cell-redirecting domains. Science Advances. https://doi.org/10.112 6/sciadv.abn4613
Multiple resolution—JaLC. (n.d.). Retrieved Jun. 27, 2024, from https://doi.org/10.15017/1931748
Ji Yi H K. (2021). Mouse anti-human IgG monoclonal antibody (Patent No. CN:112010981:B). In patent (No. CN:112010981:B). https://patentimages.storage.googleapis.com/72/3d/18/159b852f4b070e/CN112010981B.pdf

What is claimed is:

1. A heterologous polypeptide comprising:
at least one immunoglobulin binding domain; and
at least one immune cell surface protein-binding domain;
wherein the at least one immunoglobulin binding domain is derived from an Fc receptor or Fc binder, including but not limited to, FcγRIII, mFcγRIV, FcγRIIa, FcγRIIb, FcγRIIc, FcγRI, mFcγRIII, mFcγRIIa, mFcγRIIb, mFcγRI, FcαRI, C1q, FcRL, FcRL5, pIgR, Fcα/μR, FcμR, FcεRI, FcεRII, FcRn, TRIM21, allotypes, derivatives and analogs thereof;
wherein the at least one immunoglobulin binding domain is derived from an antigen-binding domain, antibody or antigen-binding fragment, variants, derivatives or analogs thereof comprising VH and VL pairs, ScFv, Fab, IgG, sdAb-VL, sdAb-VH, VHH or avimer, derivatives or analogs thereof,
wherein the heterologous polypeptide comprises one or more half-life extension domains comprising anti-HSA antigen-binding domain, antibody or antigen-binding fragment, variants, derivatives or analogs thereof comprising VHH or single domain antibodies, immunoglobulin IgG Fc domains their variants, derivatives or analogs thereof;
wherein the heterologous polypeptide comprises at least two immunoglobulin binding domains; and
wherein the heterologous polypeptide comprises at least two immune cell surface protein-binding domains.

2. A heterologous polypeptide comprising:
at least one immunoglobulin binding domain; and
at least one immune cell surface protein-binding domain;
wherein the at least one immunoglobulin binding domain comprises all or a portion of the Fc receptor of SEQ ID NO: 1, 2, 3, 4, 5, 6, 7, 8, 9, 10, 11, 12, 13, 14, 15, 16, 17, 18, 19, 20, 23, 77, 80, 271, 272, 273, 274, 275, 276, 277, 294, 296, 298, 300, 324 and 325;
wherein the at least one immunoglobulin binding domain is derived from FcγRIIa comprising one or more mutations comprising R56H, K118N, T120V, L160Q and V172E of SEQ ID NO: 9; or
wherein the at least one immunoglobulin binding domain is derived from FcγRIII comprising one or more mutations comprising S181P, K122N, T124V, Q176E, 190R, T118K, A119L and Y134F of SEQ ID NO: 1;
wherein the heterologous polypeptide comprises at least two immunoglobulin binding domains; and
wherein the heterologous polypeptide comprises at least two immune cell surface protein-binding domains.

3. A heterologous polypeptide comprising:
at least one immunoglobulin binding domain; and
at least one immune cell surface protein-binding domain;
wherein the at least one immunoglobulin binding domain is derived from an antigen-binding domain, antibody or antigen-binding fragment their variants, derivatives or analogs thereof, including but not limited to, VH and VL pairs, ScFv, Fab, IgG, sdAb-VL, sdAb-VH, VHH or avimer, their derivatives or analogs thereof,
wherein the at least one immunoglobulin binding domain comprises at least one of the CDR or FR regions in SEQ ID NO: 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 601, 602, 603, 604, 605,606, 607, 608, 609, 610, 611,612, 613, 614, 615, 616, 617, 618, 619, 620, 621, 622, 623, 624, 625, 626, 627, 628, 629, 6230, 631, 632, 633, 634, 635, 636, 637, 638, 639, 640, 641, 642, 643, 644, 645, 646, 647, 648, 649, 650, 651, 652, 653, 654, 655, 656, 657, or the avimer defined in SEQ ID NO: 132;
wherein the heterologous polypeptide comprises at least two immunoglobulin binding domains; and
wherein the heterologous polypeptide comprises at least two immune cell surface protein-binding domains.

4. A heterologous polypeptide comprising:
at least one immunoglobulin binding domain;
at least one immune cell surface protein-binding domain;
wherein at least one immune cell surface protein-binding domain is derived from an antigen-binding domain, antibody or antigen-binding fragment their variants, derivatives or analogs thereof, including but not limited to, VH and VL pairs, ScFv, Fab, IgG, sdAb-VL, sdAb-VH, VHH, their derivatives or analogs thereof;
wherein the immune cell surface protein-binding domain comprises one or more CDR or FR regions in SEQ ID NO: 580, 606, 608, 609, 610, 611, 612, 615, 626, 635, 636, 637, 638, 639, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705,706, 707, 708, 709, 710, 711,712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 840, 841, 842, 843, 844, 845, 846, 847, 848, 849, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911,912, 913, 914, 915, 916, 917, 918, 919, 920, 921, 922, 923, 924, 925, 926, 927, 928, 929, 940, 941, 942, 943, 944, 945, 946, 947, 948, 949, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046 and 1047;
wherein the heterologous polypeptide comprises at least two immunoglobulin binding domains; and
wherein the heterologous polypeptide comprises at least two immune cell surface protein-binding domains.

5. A heterologous polypeptide comprising:
at least one immunoglobulin binding domain;
at least one immune cell surface protein-binding domain;
at least one half-life extension domain;
wherein the at least one half-life extension domain comprises anti-HSA antigen-binding domain, antibody or antigen-binding fragment their variants, derivatives or analogs thereof comprising VHH or single domain antibodies, comprising one or more CDR or FR regions in SEQ ID NO: 558, 559, 560, 561, 562, 563, 564, 565, 566, 567 and 568;
wherein the heterologous polypeptide comprises at least two immunoglobulin binding domains; and
wherein the heterologous polypeptide comprises at least two immune cell surface protein-binding domains.

6. A heterologous polypeptide comprising:
at least one immunoglobulin binding domain;
at least one immune cell surface protein-binding domain;
at least one half-life extension domain;
wherein the heterologous polypeptide is a single chain of the structure:
D1-D2-D3
and
wherein the at least one half-life extension domain comprises anti-HSA antigen-binding domain, antibody or antigen-binding fragment their variants, derivatives or analogs thereof comprising VHH or single domain antibodies, comprising one or more CDR or FR regions in SEQ ID NO: 558, 559, 560, 561, 562, 563, 564, 565, 566, 567 and 568.

7. The heterologous polypeptide of claim 5, further comprising one or more linkers between the domains.

8. A heterologous polypeptide comprising:
at least two immunoglobulin binding domain;
at least one immune cell surface protein-binding domain;
at least one half-life extension domain;
wherein the heterologous polypeptide is a single chain with at least four domains of the structure:
D1-D2-D3-D4
and
wherein the at least one half-life extension domain comprises anti-HSA antigen-binding domain, antibody or antigen-binding fragment their variants, derivatives or analogs thereof comprising VHH or single domain antibodies, comprising one or more CDR or FR regions in SEQ ID NO: 558, 559, 560, 561, 562, 563, 564, 565, 566, 567 and 568.

9. The heterologous polypeptide of claim 6, further comprising one or more linkers between the domains.

10. A multimeric protein wherein regions of the molecule comprise:
at least one immunoglobulin binding domain;
at least one immune cell surface protein-binding domain; and
two half-life extension domains comprising a first Fc polypeptide and a second Fc polypeptide derived from an immunoglobulin wherein the Fc does not substantially bind the immunoglobulin binding domain;
wherein the first Fc polypeptide and the second Fc polypeptide comprise a hetero-multimerization domain wherein the hetero-multimerization domain is at least one knob into hole mutation;
wherein the multimeric protein comprises at least two immunoglobulin binding domains; and
wherein the multimeric protein comprises at least two immune cell surface protein-binding domains.

11. A multimeric protein wherein regions of the molecule comprise:
at least one immunoglobulin binding domain;
at least one immune cell surface protein-binding domain;
two half-life extension domains comprising a first Fc polypeptide and a second Fc polypeptide derived from an immunoglobulin wherein the Fc does not substantially bind the immunoglobulin binding domain by selection of a IgG heavy chain Fc polypeptide comprising mutations L234A, L235A and P329A or P329G in the constant heavy chain domain 2 (EU Numbering);
wherein the first Fc polypeptide and the second Fc polypeptide comprise a hetero-multimerization domain wherein the hetero-multimerization domain is at least one knob into hole mutation;
wherein the multimeric protein comprises at least two immunoglobulin binding domains; and
wherein the multimeric protein comprises at least two immune cell surface protein-binding domains.

12. A multimeric protein wherein regions of the molecule comprise:
at least one immunoglobulin binding domain;
at least one immune cell surface protein-binding domain;
two half-life extension domains including but not limited to a first Fc polypeptide and a second Fc polypeptide derived from an immunoglobulin wherein the Fc does not substantially bind said immunoglobulin binding domain by selection of a IgG heavy chain Fc polypeptide comprising mutations L234A, L235A and P329A or P329G in the constant heavy chain domain 2 (EU Numbering);
wherein the first Fc polypeptide comprises mutation T366W; and
wherein the second Fc polypeptide comprises T366S, L368A and Y407V in the constant heavy chain domain 3 (EU Numbering);
wherein the first Fc polypeptide further comprises S354C and wherein the second Fc polypeptide further comprises Y349C in the constant heavy chain domain 3 (EU Numbering);
wherein the multimeric protein comprises at least two immunoglobulin binding domains; and
wherein the multimeric protein comprises at least two immune cell surface protein-binding domains.

13. A multimeric protein wherein regions of the molecule comprise:
at least one immunoglobulin binding domain;
at least one immune cell surface protein-binding domain;
at least one immunoglobulin kappa or lambda constant light chain, their variants, derivatives and analogs thereof;
at least one immunoglobulin constant heavy chain domain 1 and at least a portion of the immunoglobulin hinge region, their variants, derivatives and analogs thereof;
wherein the one or more constant heavy chain domain 1 and at least a portion of the hinge comprises mutation C233S and constant light chain C214S (Kabat Numbering);
wherein the one or more constant heavy chain domain 1 and at least a portion of the hinge comprises mutation C233S and F174C and constant light chain C214S and S176C (Kabat Numbering);
wherein at least a first pair of constant heavy chain and at least a portion of the hinge that comprises no mutation at C233 and no mutation in constant light chain at C214 and at least a second pair of constant heavy chain domain 1 at least a portion of the hinge comprises mutation C233S or the like and constant light chain C214S or the like (Kabat Numbering);
wherein at least a first pair of constant heavy chain and at least a portion of the hinge that contain no mutation at C233 and no mutation in constant light chain at C214 and at least a second pair of constant heavy chain domain 1 and at least a portion of the hinge comprises mutation C233S and F174C and the constant light chain comprises mutation C214S and S176C (Kabat Numbering);
wherein at least a first pair of constant heavy chain domain 1 and at least a portion of the hinge comprises mutation C233S and constant light chain C214S and at least a second pair of constant heavy chain domain 1 and at least a portion of the contains mutation C233S and F174C and constant light chain C214S and S176C (Kabat Numbering);
wherein the heterologous polypeptide comprises one or more half-life extension domains comprising anti-HSA antigen-binding domain, antibody or antigen-binding fragment, variants, derivatives or analogs thereof comprising VHH or single domain antibodies, immunoglobulin IgG Fc domains their variants, derivatives or analogs thereof;
wherein the multimeric protein comprises at least two immunoglobulin binding domains;
and
wherein the multimeric protein comprises at least two immune cell surface protein-binding domains.

14. A multimeric protein wherein a region or regions of the molecule comprise:
at least two immunoglobulin binding domain;
at least one immune cell surface protein-binding domain;
at least a second immunoglobulin-binding domain wherein the second immunoglobulin binding domain is separated from the first immunoglobulin binding domain by a linker from 1 amino acid to 20 amino acids comprising regions of the human constant heavy chain domain 1, kappa chain domain, lambda chain domain, polypeptides comprising linkers 13 amino acids or less, linkers comprising 6 amino acids or less, the constant heavy chain domain 1 derived spacer ASTKGPSVFPLAP, ASTKGP or ASTKGPSVFPLAS, the constant kappa chain derived spacer RTVAAPSVFIFPP or RTVAAP, the constant lambda chain derived spacer SQPKAAPSVTLFP, GQPKANPTVTLFP, GQPKAAPSVTLFP, SQPKAA, GQPKAN or GQPKAA, (GGGS)₁, (GGGS)₂, (GGGS)₃, (GGGS)₄;
wherein at least one immunoglobulin binding domain comprises one or more CDR or FR regions in SEQ ID NO: 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 601, 602, 603, 604, 605,606, 607, 608, 609, 610, 611,612, 613, 614, 615, 616, 617, 618, 619, 620, 621, 622, 623, 624, 625, 626, 627, 628, 629, 6230, 631, 632, 633, 634, 635, 636, 637, 638, 639, 640, 641, 642, 643, 644, 645, 646, 647, 648, 649, 650, 651, 652, 653, 654, 655, 656, 657, or the avimer defined in SEQ ID NO: 132; and
wherein at least a second immunoglobulin-binding domain wherein the second immunoglobulin-binding domain is the same as the first immunoglobulin-binding domain; or
wherein at least a second immunoglobulin-binding domain wherein the second immunoglobulin-binding domain is different from the first immunoglobulin-binding domain.

15. A multimeric protein wherein at least one region comprises:
at least one immunoglobulin binding domain;
at least one immune cell surface protein binding domain;
one or more amino acid mutations in one or more constant or framework domains of human IgG1, IgG2, IgG3 or IgG4, variants, derivatives and analogs thereof wherein the immunoglobulin binding domain does not substantially bind itself or another region or regions of the molecule;
wherein a mutation of one or more amino acids in the constant heavy chain domain 1 comprising F122Y, P126S and K213E (Kabat Numbering);
wherein a mutation of one or more amino acids in a in the constant heavy chain domain 2 comprising N276K, L309V, L234A, L235A and P329A or P329G (EU Numbering);
wherein a heterologous polypeptide or multimeric protein and nucleic acids encoding the same, comprises at least two immunoglobulin binding domains; and
wherein a heterologous polypeptide or multimeric protein and nucleic acids encoding the same, comprises at least two immune cell surface protein binding domains.

16. A multimeric protein wherein regions of the molecule comprise:
at least one immunoglobulin kappa or lambda constant light chain, their variants, derivatives and analogs thereof, and
at least one immunoglobulin constant heavy chain domain 1 and at least a portion of the immunoglobulin hinge region, their variants, derivatives and analogs thereof, and
two half-life extension domains comprising a first Fc polypeptide and a second Fc polypeptide derived from an immunoglobulin wherein the first and second Fc polypeptides comprise a hetero-multimerization domain wherein the hetero-multimerization domain is selected from at least one knob into hole mutation;
wherein the first Fc polypeptide comprises T366W and wherein the second Fc polypeptide comprises T366S, L368A and Y407V in the constant heavy chain domain 3 (EU Numbering);
wherein the Fc does not substantially bind one or more of its cognate Fc receptors by selection of IgG heavy chain Fc polypeptide comprising L234A, L235A and P329A in the constant heavy chain domain 2 (EU Numbering);
wherein one or more constant heavy chain domain 1 and all, none or a portion of the hinge comprises mutation C233S and constant light chain C214S (Kabat Numbering);
wherein one or more constant heavy chain domain 1 and at least a portion of the hinge comprises mutation C233S and F174C and constant light chain C214S and S176C (Kabat Numbering);
wherein at least a first pair of constant heavy chain and at least a portion of the hinge that comprises no mutation at C233 and no mutation in constant light chain at C214 and at least a second pair of constant heavy chain domain 1 and at least a portion of the hinge comprises a mutation at C233S and a constant light chain C214S (Kabat Numbering);
wherein at least a first pair of constant heavy chain and at least a portion of the hinge that contain no mutation at C233 and no mutation in constant light chain at C214 and at least a second pair of constant heavy chain domain 1 and at least a portion of the hinge comprises mutations at C233S and F174C and the constant light chain comprises mutation C214S and S176C (Kabat Numbering);
wherein at least a first pair of constant heavy chain domain 1 and at least a portion of the hinge comprises a mutation at C233S and constant light chain C214S and at least a second pair of constant heavy chain domain 1 and at least a portion of the hinge contains mutation C233S and F174C and constant light chain C214S and S176C (Kabat Numbering).

17. The multimeric protein of claim 16
wherein the first Fc polypeptide further comprises S354C and wherein the second Fc polypeptide further comprises Y349C in the constant heavy chain domain 3 (EU Numbering); and
wherein IgG heavy chain Fc polypeptide further comprises P329G in the constant heavy chain domain 2 (EU Numbering).

18. A heterologous polypeptide or multimeric protein of claim 1 wherein an additional region of the molecule comprises a free cysteine at or near the C-terminus.

19. A heterologous polypeptide or multimeric protein of claim 1 wherein an additional region of the molecule comprises a covalently linked PEG-lipid.

20. A method of treatment comprising administering the heterologous polypeptide or multimeric protein of claim 1 to a patient in need thereof to treat disease as a monotherapy.

21. A method of treatment comprising administering the heterologous polypeptide or multimeric protein of claim 1 to a subject in need thereof to treat cancer, immune disorders, or pathogenic infections in combination with at least one selected from standard, current, or experimental therapeutics.

22. A nucleotide encoding the heterologous polypeptide or multimeric protein of claim 1.

23. A kit comprising the heterologous polypeptide or multimeric protein of claim 1.
